SEQUENCE LISTING (2 sequences)

SEQ ID NO: 1 - DNA, 132 bp, Unknown
  source: /note=“Description of Unknown hemoglobin UTR sequence”
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 2 - DNA, 119 bp, Homo sapiens
gaccgggcattgcccccagtctcccccaaatttgggcattgtccccgggtcttccaacggactgggcgttgctcccggacactgaggactggccccggggtctcgctcaccttcagcag

(19) United States
(12) Patent Application Publication
(10) Pub. No.: US 2019/0161730 A1
(43) Pub. Date: May 30, 2019

(54) COMPOSITIONS AND METHODS RELATED TO THERAPEUTIC CELL SYSTEMS EXPRESSING EXOGENOUS RNA

(71) Applicant: RUBIUS THERAPEUTICS, INC., Cambridge, MA (US)

(72) Inventors: Avak Kahvejian, Lexington, MA (US); Jordi Mata-Fink, Somerville, MA (US); Robert J. Deans, Riverside, CA (US); Omid Harandi, Newton, MA (US); Urjeet Khanwalkar, Cambridge, MA (US); Sneha Hariharan, Arlington, MA (US)

(21) Appl. No.: 16/315,967

(22) PCT Filed: Jul. 7, 2017
(86) PCT No.: PCT/US2017/041155

§ 371 Date: Jan. 7, 2019

Related U.S. Application Data
(60) Provisional application No. 62/359,416, filed on Jul. 7, 2016.

(51) Int. Cl. C12N 5/078; G01N 33/569; C12N 15/85

(52) U.S. Cl. CPC C12N 5/0641; G01N 33/56966; C12N 5/0644; C12N 15/85; C12N 2510/02; G01N 2333/922; G01N 2333/81; C12N 2330/51; C12N 2015/8518

(57) ABSTRACT

The invention includes compositions and methods related to erythroid cells comprising exogenous RNA encoding a protein. The exogenous RNA can comprise a heterologous untranslated region comprising a regulatory element. Alternatively or in combination, the exogenous RNA can comprise chemical modifications.

### RELATED APPLICATIONS

This application claims priority to U.S. Ser. No. 62/359,416 filed Jul. 7, 2016, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 7, 2017, is named R2081-7015WO_SL.txt and is 921 bytes in size.

### BACKGROUND

Red blood cells have been considered for use as drug delivery systems, e.g., to degrade toxic metabolites or inactivate xenobiotics, and in other biomedical applications. There is a need in the art for improved red blood cell based drug delivery systems.

### SUMMARY OF THE INVENTION

The invention includes compositions and methods related to erythroid cells comprising exogenous RNA (e.g., exogenous RNA encoding a protein). The exogenous RNA can comprise a coding region and a heterologous untranslated region (UTR), e.g., a UTR comprising a regulatory element. Alternatively or in combination, the exogenous RNA can comprise chemical modifications. Alternatively or in combination, the exogenous RNA can be a regulatory RNA such as a miRNA. While not wishing to be bound by theory, in some embodiments the exogenous RNA has improved parameters, such as stability or increased translation, relative to a control.

In certain aspects, the present disclosure provides an enucleated erythroid cell comprising: an exogenous mRNA comprising a coding region operatively linked to a heterologous untranslated region (UTR).

In certain aspects, the present disclosure provides an enucleated erythroid cell, comprising: an exogenous mRNA comprising a coding region operatively linked to a heterologous untranslated region (UTR), wherein the heterologous UTR comprises a regulatory element.

In certain aspects, the present disclosure provides an erythroid cell, e.g., an enucleated erythroid cell, comprising an exogenous mRNA that comprises one or more chemically modified nucleotides (e.g., one or more chemically modified nucleotides of Table 1, one or more chemical backbone modifications of Table 2, one or more chemically modified caps of Table 3, or any combination thereof).

The disclosure also provides a method of producing an erythroid cell, e.g., enucleated erythroid cell, comprising:

a) contacting an erythroid cell, e.g., a nucleated erythroid cell, with an exogenous mRNA comprising a coding region operatively linked to a heterologous UTR comprising a regulatory element, (e.g., isolated RNA or in vitro transcribed RNA), and

b) maintaining the contacted erythroid cell under conditions suitable for uptake of the exogenous mRNA,

thereby producing the erythroid cell, e.g., an enucleated erythroid cell.

The disclosure also provides a method of producing an erythroid cell, e.g., enucleated erythroid cell, comprising:

a) contacting an erythroid cell, e.g., a nucleated erythroid cell, with an exogenous mRNA comprising one or more chemically modified nucleotides of Table 1, one or more chemical backbone modifications of Table 2, one or more chemically modified caps of Table 3, or any combination thereof; and

b) maintaining the contacted erythroid cell under conditions suitable for uptake of the exogenous mRNA,

thereby producing the erythroid cell, e.g., an enucleated erythroid cell.

The disclosure further provides a method of producing an exogenous protein in an enucleated erythroid cell:

a) providing an erythroid cell, e.g., a nucleated erythroid cell, comprising an exogenous mRNA comprising a coding region operatively linked to a heterologous UTR comprising a regulatory element, (e.g., isolated RNA or in vitro transcribed RNA), and

b) culturing the erythroid cell under conditions suitable for production of the exogenous protein,

thereby producing the exogenous protein.

The disclosure further provides a method of producing an exogenous protein in an enucleated erythroid cell:

a) providing an erythroid cell, e.g., a nucleated erythroid cell, comprising one or more chemically modified nucleotides of Table 1, one or more chemical backbone modifications of Table 2, one or more chemically modified caps of Table 3, or any combination thereof, and

b) culturing the erythroid cell under conditions suitable for production of the exogenous protein,

thereby producing the exogenous protein.

In certain aspects, the disclosure provides a method of providing a subject with an exogenous protein, providing a subject with an enucleated erythroid cell which can produce an exogenous protein, or treating a subject, comprising administering to the subject:

an erythroid cell, e.g., a nucleated erythroid cell, comprising an exogenous mRNA comprising a coding region operatively linked to a heterologous UTR comprising a regulatory element, (e.g., isolated RNA or in vitro transcribed RNA), thereby providing a subject with an exogenous protein, providing a subject with an enucleated erythroid cell which can produce an exogenous protein, or treating a subject.

In certain aspects, the disclosure provides a method of providing a subject with an exogenous protein, providing a subject with an enucleated erythroid cell which can produce an exogenous protein, or treating a subject, comprising administering to the subject:

an erythroid cell, e.g., a nucleated erythroid cell, comprising an exogenous mRNA comprising one or more chemically modified nucleotides of Table 1, one or more chemical backbone modifications of Table 2, one or more chemically modified caps of Table 3, or any combination thereof,

thereby providing a subject with an exogenous protein, providing a subject with an enucleated erythroid cell which can produce an exogenous protein, or treating a subject.

In some aspects, the disclosure provides a method of evaluating an erythroid cell, e.g., enucleated erythroid cell (or a batch of such cells) comprising:

a) providing an erythroid cell, e.g., a nucleated erythroid cell, comprising an exogenous mRNA comprising a coding region operatively linked to a heterologous UTR comprising a regulatory element (or a batch of such cells), and

b) evaluating the erythroid cell, e.g., the nucleated erythroid cell (or batch of such cells) for a preselected parameter,

thereby evaluating the erythroid cell, e.g., enucleated erythroid cell (or a batch of such cells).

In some aspects, the disclosure provides a method of evaluating an erythroid cell, e.g., enucleated erythroid cell (or a batch of such cells) comprising:

a) providing an erythroid cell, e.g., a nucleated erythroid cell, comprising one or more chemically modified nucleotides of Table 1, one or more chemical backbone modifications of Table 2, one or more chemically modified caps of Table 3, or any combination thereof, and

b) evaluating the erythroid cell, e.g., the nucleated erythroid cell (or batch of such cells) for a preselected parameter,

thereby evaluating the erythroid cell, e.g., enucleated erythroid cell (or a batch of such cells).

In certain aspects, the present disclosure provides a method of producing a plurality of enucleated erythroid cells comprising an exogenous protein, comprising: a) contacting an erythroid cell with an exogenous mRNA comprising a coding region and a heterologous UTR, (e.g., isolated RNA or in vitro transcribed RNA), and b) culturing the erythroid cell under conditions suitable for production of the exogenous protein, thereby producing the enucleated erythroid cell comprising the exogenous protein.

In some aspects, the present disclosure provides an enucleated erythroid cell comprising an exogenous mRNA comprising one or more chemically modified nucleotides of Table 1, one or more chemical backbone modifications of Table 2, or one or more chemically modified caps of Table 3, or any combination thereof.

In some aspects, the present disclosure provides a method of producing a plurality of enucleated erythroid cells comprising an exogenous protein, comprising:

a) contacting an erythroid cell with an exogenous mRNA described herein (e.g., isolated RNA or in vitro transcribed RNA) that encodes an exogenous protein, wherein the exogenous mRNA comprises one or more chemically modified nucleotides of Table 1, one or more chemical backbone modifications of Table 2, or one or more chemically modified caps of Table 3, or any combination thereof, and

b) culturing the erythroid cell under conditions suitable for production of the exogenous protein,

thereby producing the enucleated erythroid cell comprising the exogenous protein.

In certain aspects, the present disclosure provides an RNA molecule comprising: a) a coding region that encodes a red blood cell transmembrane protein, e.g., GPA or Kell, and b) a heterologous UTR, e.g., a UTR comprising one or more regulatory elements or a hemoglobin 3′ UTR.

In certain aspects, the present disclosure provides an RNA molecule comprising: a) a coding region that encodes a red blood cell transmembrane protein, e.g., GPA or Kell, and b) one or more modified nucleotides described herein, e.g., a nucleotide of Table 1, 2, or 3.

In some aspects, the present disclosure provides a method of producing an erythroid cell described herein, providing contacting an erythroid cell, e.g., an erythroid cell precursor, with one or more nucleic acids described herein and placing the cell in conditions that allow expression of the nucleic acid.

In some aspects, the present disclosure provides a preparation, e.g., pharmaceutical preparation, comprising a plurality of erythroid cells described herein, e.g., at least 10⁸, 10⁹, 10¹⁰, 10¹¹, or 10¹² cells.

In some aspects, the disclosure provides a method of contacting erythroid cells with an exogenous mRNA during maturation phase, e.g., during day 1, 2, 3, 4, 5, 6, 7, 8, 9, 10, 11, 12, 13, or 14 of maturation phase.

Any of the aspects herein, e.g., the aspects above, can be characterized by one or more of the embodiments herein, e.g., the embodiments below.

In some embodiments, the methods herein comprise a step of:

c) culturing the erythroid cell subsequent to uptake of the exogenous RNA,

In embodiments, the UTR occurs naturally operatively linked to a coding region other than the subject coding region, or has at least at least 70, 80, 90, 95, 99, or 100% homology to such naturally occurring UTR. In embodiments, the UTR does not occur naturally with the subject coding region, e.g., differs by at least 1 nucleotide, e.g., by at least 1, 2, 3, 4, 5, 10, 20, 25, 30, 35, 40, 45, or 50% of its nucleotides, from the UTR which occurs naturally operatively linked with the subject coding region. In embodiments, the UTR does not exist in nature.

In embodiments, the UTR comprises a 3′ UTR. In embodiments, the UTR comprises a 5′ UTR. In embodiments, the heterologous UTR is a 5′ UTR, and the exogenous mRNA further comprises a heterologous 3′ UTR. In embodiments, the UTR comprises a region that corresponds to an intron. In some embodiments, the RNA is capable of undergoing alternative splicing, e.g., encodes a plurality of splice isoforms. In embodiments, the alternative splicing comprises exon skipping, alternative 5′ donor site usage, alternative 3′ acceptor site usage, or intron retention. In an embodiment, the UTR comprises an intron in the coding region. In an embodiment, an intron in the coding region comprises the UTR. In an embodiment, the UTR is a 5′ UTR that comprises an intron.

In embodiments, the enucleated erythroid cell further comprises a second UTR. In embodiments, the enucleated erythroid comprises a 3′ UTR and a 5′ UTR.

In embodiments, the UTR occurs naturally in a wild-type human cell. In embodiments, the UTR does not occur naturally in a wild-type human cell.

In embodiments, the coding region occurs naturally in a wild-type human cell and/or encodes a protein that occurs naturally in a wild-type human cell. In embodiments, the coding region does not occur naturally in a wild-type human cell and/or encodes a protein that does not occur naturally in a wild-type human cell. In embodiments, the UTR occurs naturally operatively linked with a coding region that is expressed in a wild-type erythroid cell, e.g., a hemoglobin coding region.

In embodiments, the UTR is a globin UTR, e.g., a hemoglobin UTR, e.g., having the sequence of SEQ ID NO: 1 or a sequence with at least 70%, 75%, 80%, 85%, 90%, 95%, 96%, 97%, 98%, or 99% identity thereto.

In embodiments, the coding region encodes an enzyme, antibody molecule, complement regulatory protein, chelator, or a protein listed in Table 4. In some embodiments, the exogenous polypeptide comprises phenylalanine ammonia lyase (PAL) or a phenylalanine-metabolizing fragment or variant thereof.

In embodiments, the cell further comprises a protein encoded by the exogenous mRNA. In embodiments, the cell does not comprise DNA encoding the exogenous mRNA.

In some embodiments, the cell has not been or is not hypotonically loaded.

In embodiments, the exogenous mRNA comprises one or more chemically modified nucleotides, chemical backbone modifications, or modified caps, or any combination thereof. In embodiments, at least 50%, 60%, 70%, 80%, or 85% of the cells in the plurality produce the exogenous protein. In embodiments, the cell population has at least 50%, 60%, 70%, 80%, 85%, 90%, or 95% cell viability.

In embodiments, the method comprises performing transfection, electroporation, hypotonic loading, change in cell pressure, cell deformation (e.g., CellSqueeze), or other method for disrupting the cell membrane to allow the exogenous RNA to enter the cell.

In embodiments, the exogenous mRNA has a half-life that is at least 10%, 20%, 30%, 40%, 50%, 60%, 70%, 80%, or 90% greater, or 2-fold, 5-fold, 10-fold, 20-fold, or 100-fold greater than the half-life of a corresponding mRNA lacking the chemical modification in a similar erythroid cell. In embodiments, the exogenous mRNA is present in the erythroid cell at a level that is at least 10%, 20%, 30%, 40%, 50%, 60%, 70%, 80%, or 90% greater, or 2-fold, 5-fold, 10-fold, 20-fold, or 100-fold greater than the level of an mRNA of identical sequence that lacks the chemical modification, in an otherwise similar erythroid cell, when measured at a similar timepoint after introduction of the mRNA.

In embodiments, the exogenous protein is present in the erythroid cell at a level that is at least 10%, 20%, 30%, 40%, 50%, 60%, 70%, 80%, or 90% greater, or 2-fold, 5-fold, 10-fold, 20-fold, or 100-fold greater than the level of protein produced by an mRNA of identical sequence that lacks the chemical modification, in an otherwise similar erythroid cell, when measured at a similar timepoint after introduction of the mRNA. In embodiments, the timepoint is 1, 2, 3, 4, 5, 6, 7, 14, 21, or 28 days after the cell is contacted with the mRNA. In embodiments, the chemical modification comprises a pseudouridine. In embodiments, the mRNA further comprises a cap. In embodiments, the mRNA further comprises a polyA tail;

In embodiments, the exogenous protein is present in the erythroid cell at a level that is at least 10%, 20%, 30%, 40%, 50%, 60%, 70%, 80%, or 90% greater, or 2-fold, 5-fold, 10-fold, 20-fold, or 100-fold greater than the level of protein produced by an mRNA of identical sequence that lacks a polyA tail, in an otherwise similar erythroid cell, when measured at a similar timepoint after introduction of the mRNA. In embodiments, the timepoint is 1, 2, 3, 4, 5, 6, 7, 14, 21, or 28 days after the cell is contacted with the mRNA.

In embodiments, a cell described herein comprises a heterologous UTR, e.g., a heterologous UTR comprising a regulatory element, and further comprises a chemical modification, e.g., comprises one or more chemically modified nucleotides of Table 1, one or more chemical backbone modifications of Table 2, one or more chemically modified caps of Table 3, or any combination thereof.

In some embodiments, a contacting step described herein (e.g., contacting the erythroid cell with mRNA) occurs before enucleation of the cell, and in other embodiments, the contacting step occurs after enucleation of the cell. In embodiments, the contacting step is performed on a population of cells comprising a plurality of enucleated cells and a plurality of nucleated cells. The population of cells may be, e.g., primarily nucleated or primarily enucleated. In embodiments, the method comprises culturing the cells under conditions suitable for enucleation.

In some embodiments of any of the methods herein, providing comprises contacting an erythroid cell, e.g., a nucleated erythroid cell, with an exogenous mRNA comprising a coding region operatively linked to a heterologous UTR comprising a regulatory element, (e.g., isolated RNA or in vitro transcribed RNA). In embodiments, providing comprises receiving the erythroid cell from another entity.

In embodiments, the parameter described herein is selected from: the ability to express the exogenous protein; the structure or function of the exogenous protein; the proportion of cells comprising the endogenous mRNA; the proportion of cells comprising the endogenous protein; the level of exogenous mRNA in the cell; the level of exogenous protein in the cell; cell proliferation rate; or cell differentiation state. In embodiments, the method comprises comparing a value for the preselected parameter with a reference. In embodiments, the method comprises, responsive to the value for the parameter, or a comparison of the value with a reference, classifying, approving, or rejecting the cell or batch of cells.

In some embodiments, a cell described herein is disposed in a population of cells. In embodiments, the population of cells comprises a plurality of cells as described herein, and optionally further comprises one or more other cells, e.g., wild-type erythroid cells that lack the exogenous mRNA, nucleated erythroid cells, or non-erythroid cells. In embodiments, the population of cells comprises at least a first cell comprising a first exogenous RNA and a second cell comprising a second exogenous RNA. In embodiments, the population of cells comprises at least a first cell comprising a first exogenous RNA and a second exogenous RNA.

In embodiments, the RNA is produced by in vitro transcription or solid phase chemical synthesis.

In some embodiments, e.g., in embodiments involving contacting erythroid cells with an exogenous mRNA during maturation phase, the contacting comprises electroporation. In some embodiments, the contacting is performed at between days 6-8, 5-9, 4-10, 3-11, 2-12, or 1-13 of maturation. In some embodiments, the contacting is performed on or after day 1, 2, 3, 4, 5, 6, 7, 8, 9, 10, 11, 12, 13, or 14 of maturation. In some embodiments, the method further comprises culturing the cells for at least 1, 2, 3, 4, 5, 6, 7, 8, 9, 10, 15, or 20 days after the contacting. In some embodiments, the method further comprises testing expression of the transgenic mRNA, e.g., detecting a level of a protein encoded by the transgenic mRNA, after the contacting, e.g., at 1, 2, 3, 4, 5, 6, 7, 8, 9, 10, 11, 12, 13, or 14 days after contacting the cell with the mRNA.

In some aspects, the disclosure provides a method of making an erythroid cell comprising an mRNA encoding an exogenous protein, comprising:

a) providing an erythroid cell in maturation phase, and

b) contacting (e.g., electroporating) the erythroid cell with an mRNA encoding the exogenous protein, under conditions that allow uptake of the mRNA by the erythroid cell,

thereby making an erythroid cell comprising an mRNA encoding an exogenous protein.

In embodiments, the method comprises providing a population of erythroid cells in maturation phase and contacting the population with the mRNA encoding the exogenous protein. In embodiments, a plurality of erythroid cells of the population each takes up an mRNA encoding the exogenous protein. In embodiments the cell expresses the exogenous protein. In embodiments the cell comprises the exogenous protein. In embodiments, a plurality of cells in the population express the exogenous protein. In embodiments, the population of cells in maturation phase is a population of cells expanded in a maturation medium for 3-7 days, e.g., 4-5 or 4-6 days. In embodiments, the population of cells in maturation phase is a population described herein, e.g., having a specified percent enucleation, translational activity, or cell surface marker expression.

In embodiments, at least 50%, 60%, 70%, 80%, 85%, 90%, or 95% of the cells in the population comprise the exogenous protein, e.g., 5 days after contacting with the mRNA. In embodiments, the cells in the population comprise at least 1,000, 2,000, 5,000, 10,000, 20,000, 50,000, or 100,000 copies of the exogenous protein, e.g., 5 days after the contacting with the mRNA. In embodiments, the cells comprise at least 1,000, 2,000, 5,000, 10,000, 20,000, 50,000, or 100,000 copies of the exogenous protein for at least 5, 6, 7, 8, 9, 10, 11, 12, 13, 14, or 15 days after contacting with the mRNA. In embodiments, the cells comprise at least 1,000 copies of the exogenous protein for at least 5, 6, 7, 8, 9, 10, 11, 12, 13, 14, or 15 days after contacting with the mRNA.

In some aspects, the disclosure provides a method of making an erythroid cell comprising an mRNA encoding an exogenous protein, comprising:

a) providing an erythroid cell in maturation phase, and

b) contacting the erythroid cell with an mRNA encoding the exogenous protein, under conditions that allow uptake of the mRNA by the erythroid cell,

thereby making an erythroid cell comprising an mRNA encoding an exogenous protein.

In embodiments, the method comprises providing a population of erythroid cells in maturation phase and contacting a plurality of cells of the population of erythroid cells with the mRNA encoding the exogenous protein. In embodiments, the population of erythroid cells in maturation phase is a population of cells expanded in a maturation medium for 3-7 days, e.g., 4-5 or 4-6 days. In embodiments, the population of erythroid cells is a population of erythroid cells comprising one or more (e.g., 2, 3, 4, 5, 6, 7, 8, 9, 10, 11, 12, 13, 14, 15, 16, 17, or more) of the following properties:

i.a) 2-40%, 3-33%, 5-30%, 10-25%, or 15-20% of the cells in the population are enucleated;

i.b) greater than 0%, 0.1%, 0.2%, or 0.5%, but less than 2%, 3%, 4%, or 5% of the cells in the population are enucleated;

i.c) greater than 0%, 0.1%, 0.2%, or 0.5%, but less than 6%, 10%, 15%, 20%, or 25% of the cells in the population are enucleated;

i.d) greater than 0%, 0.1%, 0.2%, or 0.5%, but less than 30%, 35%, 40%, 45%, or 50% of the cells in the population are enucleated;

i.e) no more than 1%, 2%, 3%, 5%, 10%, 15%, or 20% of the cells in the population are enucleated;

i.f) no more than 25%, 30%, 35%, 40%, 45%, or 50% of the cells in the population are enucleated;

i.g) the population of cells has reached 6-70%, 10-60%, 20-50%, or 30-40% of maximal enucleation;

i.h) the population of cells has reached no more than 1%, 2%, 3%, 5%, 10%, 15%, or 20% of maximal enucleation;

i.i) the population of cells has reached no more than 25%, 30%, 35%, 40%, 45%, 50%, or 60% of maximal enucleation;

ii.a) the population of cells is fewer than 3, 2, or 1 population doubling from a plateau in cell division;

ii.b) the population of cells is capable of fewer than 3, 2, or 1 population doubling;

ii.c) the population will increase by no more than 1.5, 2, or 3 fold before the population reaches an enucleation level of at least 70% of cells in the population;

iii.a) at least 80%, 85%, 90%, 95%, or 99% of the cells in the population are normoblasts (e.g., polychromatic or orthochromatic normoblasts);

iii.b) at least 50%, 60%, 70%, 75%, or 79% of the cells in the population are normoblasts (e.g., polychromatic or orthochromatic normoblasts);

iii.c) 30-90%, 40-90%, 50-90%, 60-90%, or 70-90% of the cells in the population are normoblasts (e.g., polychromatic or orthochromatic normoblasts);

iii.d) at least 80%, 85%, 90%, 95%, or 99% of the cells in the population exhibit the morphology of a normoblast (e.g., a polychromatic or orthochromatic normoblast);

iii.e) at least 50%, 60%, 70%, 75%, or 79% of the cells in the population exhibit the morphology of a normoblast (e.g., a polychromatic or orthochromatic normoblast); or

iii.f) 30-90%, 40-90%, 50-90%, 60-90%, or 70-90% of the cells in the population exhibit the morphology of a normoblast (e.g., a polychromatic or orthochromatic normoblast).

In embodiments, prior to or after contacting the plurality of cells with the mRNA encoding the exogenous protein, the plurality of cells are separated from the population of erythroid cells, e.g., the plurality of cells are separated from the population based on enucleation status (e.g., the plurality of cells are nucleated cells and the rest of the population are enucleated cells).

In embodiments, prior to or after contacting the plurality of cells with the mRNA encoding the exogenous protein, the method further comprises synchronizing the differentiation stage of the population of erythroid cells, e.g., by arresting the growth, development, hemoglobin synthesis, or the process of enucleation of the population, e.g., by incubating the population with an inhibitor of enucleation (e.g., an inhibitor of histone deacetylase (HDAC), an inhibitor of mitogen-activated protein kinase (MAPK), an inhibitor of cyclin-dependent kinase (CDK), or a proteasome inhibitor). In embodiments, arresting occurs prior to enucleation of more than 1, 2, 3, 4, 5, 6, 7, 8, 9 or 10% of the cells in the population.

In some aspects, the disclosure provides a method of manufacturing a population of reticulocytes that express an exogenous protein, the method comprising:
(a) providing a population of erythroid precursor cells (e.g., CD34+ cells);
(b) culturing the population of erythroid precursor cells under differentiating conditions to provide a population of differentiating erythroid cells;
(c) contacting the differentiating erythroid cells with an mRNA encoding the exogenous protein, under conditions that allow uptake of the mRNA by the differentiating erythroid cells, wherein the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated); and
(d) further culturing the differentiating erythroid cells to provide a population of reticulocytes,

thereby manufacturing a population of reticulocytes that express the exogenous protein.

In embodiments, the further culturing comprises fewer than 3, 2, or 1 population doubling. In embodiments, the contacting is performed when at least 50% (at least 60%, 70%, 75%, 80%, 90%, or 95%) of the differentiating erythroid cells exhibit the morphology of a normoblast (e.g., a polychromatic or orthochromatic normoblast).

In some aspects, the disclosure provides a method of manufacturing a population of reticulocytes that express an exogenous protein, comprising (a) providing a population of erythroid precursor cells, (b) culturing the population of erythroid precursor cells under differentiating conditions to provide a population of differentiating erythroid cells, (c) contacting the differentiating erythroid cells with an mRNA encoding the exogenous protein, wherein the improvement comprises: the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated).

In embodiments, the contacting is performed when the population of differentiating erythroid cells has fewer than 3, 2, or 1 population doubling before a plateau in cell division. In embodiments, the contacting is performed when at least 50% (at least 60%, 70%, 75%, 80%, 90%, or 95%) of the differentiating erythroid cells exhibit the morphology of a normoblast (e.g., a polychromatic or orthochromatic normoblast).

In some aspects, the disclosure provides a method of making an erythroid cell comprising an mRNA that encodes an exogenous protein, comprising:

providing a reaction mixture comprising an erythroid cell and an mRNA encoding the exogenous protein, under conditions which inhibit degradation of mRNA, e.g., by inclusion in the reaction mixture a ribonuclease inhibitor, and

maintaining the reaction mixture under conditions that allow uptake of the mRNA by the erythroid cell,

thereby making an erythroid cell comprising an mRNA encoding an exogenous protein.

In embodiments, the method comprises providing a population of erythroid cells and contacting the population with the mRNA encoding the exogenous protein. In embodiments, a plurality of erythroid cells of the population each takes up an mRNA encoding the exogenous protein. In embodiments, the cell or plurality of cells express the exogenous protein. In embodiments, the cell or plurality of cells comprises the exogenous protein. In embodiments, the method further comprises electroporating the cell or population of cells. In embodiments, the method further comprises contacting a population of erythroid cells with a ribonuclease inhibitor. In embodiments, the method comprises contacting the population of cells with the ribonuclease inhibitor before, during, or after contacting the cells with the mRNA. In embodiments, the method comprises contacting the cells with the ribonuclease inhibitor at day 4, 5, or 6 of maturation phase. In embodiments, the cell is in maturation phase. In embodiments, the population of cells in maturation phase is a population described herein, e.g., having a specified percent enucleation, translational activity, or cell surface marker expression.

In embodiments, the mRNA is in vitro transcribed mRNA.

In embodiments, at least 80%, 85%, 90%, or 95% (and optionally up to 95%) of the cells of the population are viable (e.g., as determined by Annexin V staining) 5 days after the cells are contacted with the mRNA. In embodiments, at least 10%, 20%, 30%, 40%, 50%, 60%, 70%, 80%, or 90% of the cells of the population are enucleated 5 days after the cells are contacted with the mRNA. In embodiments, the proportion of cells that are enucleated 5 days after the cells are contacted with the mRNA is at least 50%, 60%, 70%, 80%, 90%, or 95% of the proportion of cells that are enucleated in an otherwise similar population of cells not treated with the ribonuclease inhibitor. In embodiments, the population of cells comprises at least 1×10⁶, 2×10⁶, 5×10⁶, 1×10⁷, 2×10⁷, 5×10⁷, or 1×10⁸ cells at the time the cells are contacted with the mRNA. In embodiments, the population of cells expands by at least 10%, 20%, 30%, 40%, 50%, 60%, 70%, 80%, or 90% within 5 days after the cells are contacted with the mRNA. In embodiments, at least 50%, 60%, 70%, 80%, 85%, 90%, or 95% of the cells of the population express the exogenous protein, e.g., 5 days after the cells are contacted with the mRNA. In embodiments, at least 50%, 60%, 70%, 80%, 85%, 90%, or 95% of the cells of the population comprise at least 1,000, 2,000, 5,000, 10,000, 20,000, 50,000, or 100,000 copies of the exogenous protein, e.g., 5 days after the cells are contacted with the mRNA. In embodiments, at least 50%, 60%, 70%, 80%, 85%, 90%, or 95% of the cells of the population comprise at least 1,000 copies of the exogenous protein, e.g., 5 days after the cells are contacted with the mRNA. In embodiments, at least 50%, 60%, 70%, 80%, 85%, 90%, or 95% of the cells of the population comprise at least 10,000 copies of the exogenous protein, e.g., 5 days after the cells are contacted with the mRNA. In embodiments, the population of cells comprises at least 10%, 20%, 30%, 40%, 50%, 60%, 70%, 80%, or 90% more, or at least 2-fold, 3-fold, 4-fold, or 5-fold more of the exogenous protein than an otherwise similar population of cells not treated with the ribonuclease inhibitor.

The disclosure also provides, in some aspects, a reaction mixture comprising: i) an erythroid cell, ii) an mRNA comprising an exogenous protein and iii) a ribonuclease inhibitor.

In embodiments, the mRNA is inside the erythroid cell. In embodiments, the reaction mixture comprises a plurality of erythroid cells.

The disclosure also provides, in some aspects, a method of assaying a reaction mixture comprising enucleated erythroid cells that comprise an exogenous protein for a ribonuclease inhibitor, comprising:

providing a reaction mixture comprising enucleated erythroid cells that comprise an exogenous protein,

assaying for the presence or level of a ribonuclease inhibitor, e.g., by ELISA, Western blot, or mass spectrometry, e.g., in an aliquot of the reaction mixture.

In embodiments, the method comprises comparing the level of ribonuclease inhibitor to a reference value.

In embodiments, the method further comprises, responsive to the comparison, performing one or more of:

classifying the population, e.g., as meeting a requirement or not meeting a requirement, e.g., wherein the requirement is met when the level of ribonuclease inhibitor is below the reference value,

classifying the population as suitable or not suitable for a subsequent processing step, e.g., when the population is suitable for a subsequent purification step when the level of ribonuclease inhibitor is above the reference value,

classifying the population as suitable or not suitable for use as a therapeutic, or

formulating or packaging the population, or an aliquot thereof, for therapeutic use, e.g., when the level of ribonuclease inhibitor is below the reference value.

In embodiments, the ribonuclease inhibitor is RNAsin Plus (e.g., from Promega), Protector RNAse Inhibitor (e.g., from Sigma), or Ribonuclease Inhibitor Huma (e.g., from Sigma).

The disclosure also provides, in some aspects, a method of making an erythroid cell comprising an mRNA that encodes an exogenous protein, comprising:

providing a reaction mixture comprising an erythroid cell and an mRNA encoding the exogenous protein, under conditions which inhibit protein degradation, e.g., by inclusion in the reaction mixture a protease inhibitor, e.g., a proteasome inhibitor, and

maintaining the reaction mixture under conditions that allow uptake of the mRNA by the erythroid cell,

thereby making an erythroid cell comprising an mRNA encoding an exogenous protein.

In embodiments, the method comprises providing a population of erythroid cells and contacting the population with the mRNA encoding the exogenous protein. In embodiments, a plurality of erythroid cells of the population each takes up an mRNA encoding the exogenous protein. In embodiments, the cell or plurality of cells express the exogenous protein. In embodiments, the cell or plurality of cells comprises the exogenous protein. In embodiments, the method further comprises electroporating the cell or population of cells. In embodiments, the method further comprises contacting the population of erythroid cells with a proteasome inhibitor. In embodiments, the method comprises contacting the population of cells with the proteasome inhibitor before, during, or after contacting the cells with the mRNA, e.g., 0.5-2 days before or after contacting the cells with the mRNA. In embodiments, the method comprises contacting the population of cells with the proteasome inhibitor 0.5-2 days before contacting the cells with the mRNA. In embodiments, the method comprises removing the proteasome inhibitor (e.g., by washing the cells) before electroporation.

In embodiments, the method comprises contacting the cells with the proteasome inhibitor at day 3-7 of maturation, e.g., day 4, 5, or 6 of maturation phase. In embodiments, the cell is in maturation phase. In embodiments, the population of cells in maturation phase is a population described herein, e.g., having a specified percent enucleation, translational activity, or cell surface marker expression.

In embodiments, the mRNA is in vitro transcribed mRNA. In embodiments, at least 50%, 60%, 70%, 80%, 85%, 90%, or 95% of the cells of the population are viable 5 days after the cells are contacted with the mRNA. In embodiments, at least 10%, 20%, 30%, 40%, 50%, 60%, 70%, 80%, or 90% of the cells of the population are enucleated 5 days after the cells are contacted with the mRNA. In embodiments, the proportion of cells that are enucleated 5 days after the cells are contacted with the mRNA is at least 50%, 60%, 70%, 80%, 90%, or 95% of the proportion of cells that are enucleated in an otherwise similar population of cells not treated with the proteasome inhibitor. In embodiments, the population of cells comprises at least 1×10⁶, 2×10⁶, 5×10⁶, 1×10⁷, 2×10⁷, 5×10⁷, or 1×10⁸ cells at the time the cells are contacted with the mRNA.

In embodiments, the population of cells expands by at least 10%, 20%, 30%, 40%, 50%, 60%, 70%, 80%, or 90% within 5 days after the cells are contacted with the mRNA. In embodiments, at least 50%, 60%, 70%, 80%, 85%, 90%, or 95% of the cells of the population express the exogenous protein, e.g., 5 days after the cells are contacted with the mRNA. In embodiments, at least 50%, 60%, 70%, 80%, 85%, 90%, or 95% of the cells of the population comprise at least 1,000, 2,000, 5,000, 10,000, 20,000, 50,000, or 100,000 copies of the exogenous protein, e.g., 5 days after the cells are contacted with the mRNA. In embodiments, at least 50%, 60%, 70%, 80%, 85%, 90%, or 95% of the cells of the population comprise at least 1,000 copies of the exogenous protein, e.g., 5 days after the cells are contacted with the mRNA. In embodiments, at least 50%, 60%, 70%, 80%, 85%, 90%, or 95% of the cells of the population comprise at least 10,000 copies of the exogenous protein, e.g., 5 days after the cells are contacted with the mRNA. In embodiments, the population of cells comprises at least 10%, 20%, 30%, 40%, 50%, 60%, 70%, 80%, or 90% more, or at least 2-fold, 3-fold, 4-fold, or 5-fold more of the exogenous protein than an otherwise similar population of cells not treated with the proteasome inhibitor.

In some aspects, the disclosure provides a reaction mixture comprising: i) an erythroid cell, ii) an mRNA comprising an exogenous protein and iii) a proteasome inhibitor.

In embodiments, the mRNA is inside the erythroid cell. In embodiments, the reaction mixture comprises a plurality of erythroid cells.

The disclosure also provides, in some aspects method of assaying a reaction mixture comprising enucleated erythroid cells that comprise an exogenous protein for a proteasome inhibitor, comprising:

providing a reaction mixture comprising enucleated erythroid cells that comprise an exogenous protein,

assaying for the presence or level of a proteasome inhibitor, e.g., by ELISA, Western blot, or mass spectrometry, e.g., in an aliquot of the reaction mixture.

In embodiments, the method further comprises comparing the level of proteasome inhibitor to a reference value.

In embodiments, the method further comprises, responsive to the comparison, one or more of:

classifying the population, e.g., as meeting a requirement or not meeting a requirement, e.g., wherein the requirement is met when the level of proteasome inhibitor is below the reference value,

classifying the population as suitable or not suitable for a subsequent processing step, e.g., when the population is suitable for a subsequent purification step when the level of proteasome inhibitor is above the reference value,

classifying the population as suitable or not suitable for use as a therapeutic,

formulating or packaging the population, or an aliquot thereof, for therapeutic use, e.g., when the level of proteasome inhibitor is below the reference value.

In embodiments, the proteasome inhibitor is a 20S proteasome inhibitor, e.g., MG-132 or carfilzomib, or a 26S proteasome inhibitor, e.g., bortezomib.

In embodiments, the method of making an erythroid cell comprising an mRNA encoding a first exogenous protein and a second exogenous protein, comprising:

a) providing an erythroid cell, e.g., in maturation phase, and

b) contacting the erythroid cell with an mRNA encoding the first exogenous protein and a second mRNA encoding the second exogenous protein, under conditions that allow uptake of the first mRNA and second mRNA by the erythroid cell,

thereby making an erythroid cell comprising the first mRNA and the second mRNA.

In embodiments, the erythroid cell comprises at least 1,000, 2,000, 5,000, 10,000, 20,000, 50,000, or 100,000 copies of the first exogenous protein and the second exogenous protein, e.g., 5 days after the contacting with the mRNA.

The disclosure also provides, in some aspects, a method of producing a population of erythroid cells expressing a first exogenous protein and a second exogenous protein, comprising:

a) providing a population of erythroid cells, e.g., in maturation phase, and

b) contacting the population of erythroid cells with a first mRNA encoding a first protein and a second mRNA encoding a second protein,

thereby making an erythroid cell comprising an mRNA encoding an exogenous protein

wherein at least 80%, 85%, 90%, 95%, 96%, 97%, 98%, or 99% of cells in the population comprise both of the first mRNA and the second mRNA.

In embodiments, the population of erythroid cells comprises an average of at least 1,000, 2,000, 5,000, 10,000, 20,000, 50,000, or 100,000 copies of the first exogenous protein and the second exogenous protein per cell, e.g., 5 days after the contacting with the mRNA.

In embodiments, the contacting comprises performing electroporation.

In embodiments, the population of cells comprises the first exogenous protein and the second exogenous protein in at least 80%, 85%, 90%, 95%, 96%, 97%, 98%, or 99% of cells for at least 5 days after the cells were contacted with the first and second mRNAs. In embodiments, the population of cells comprises the first exogenous protein and the second exogenous protein in at least 80%, 85%, 90%, 95%, 96%, 97%, 98%, or 99% of cells for at least 2, 4, 6, 8, 10, 12, or 14 days after the cells were contacted with the first and second mRNAs. In embodiments, the population of cells comprises the first exogenous protein and the second exogenous protein in at least 80% of cells for at least 2, 4, 6, 8, 10, 12, or 14 days after the cells were contacted with the first and second mRNAs.

In embodiments, the first exogenous protein has an amino acid length that is no more than 10%, 20%, 30%, 40%, or 50% longer than that of the second exogenous protein. In some embodiments, the average level of the second exogenous protein is no more than 10%, 20%, 30%, 40%, or 50% of the level of the first exogenous protein in the erythroid cell population.

In embodiments, the first exogenous protein has an amino acid length that is at least 50%, 60%, 70%, 80%, 90%, 2-fold, or 3-fold longer than that of the second exogenous protein. In some embodiments, the average level of the second exogenous protein is at least 50%, 60%, 70%, 80%, 90%, 2-fold, or 3-fold higher than the level of the first exogenous protein in the erythroid cell population.

The disclosure also provides, in certain aspects, a population of erythroid cells wherein at least 80%, 85%, 90%, 95%, 96%, 97%, 98%, or 99% of cells in the population express a first exogenous protein and a second exogenous protein, wherein the population was not made by contacting the cells with DNA encoding the first or second exogenous protein.

The disclosure also provides, in certain aspects, method of producing a plurality of erythroid cells comprising a predetermined number of copies of an exogenous protein per cell, comprising contacting the population with a predetermined amount of mRNA encoding the exogenous protein, thereby making the erythroid cell comprising the predetermined amount of the exogenous protein. In embodiments, the method further comprises evaluating one or more of the plurality of erythroid cells (e.g., enucleated erythroid cells) to determine the amount of the exogenous protein.

In some aspects, the disclosure provides a method of evaluating the amount of an exogenous protein in a sample of erythroid cells, e.g., enucleated erythroid cells comprising:

providing a plurality of erythroid cells comprising a predetermined number of copies of an exogenous protein per cell, which was made by contacting the population with a predetermined amount of mRNA encoding the exogenous protein, and

determining the amount of the exogenous protein in the plurality of erythroid cells.

In some embodiments, the method comprises:

contacting the cell population with 0.6±50%, ±20% or ±10% ug of mRNA per 5E6 cells in the population yields a population of cells expressing 1,000,000±50%, ±20% or ±10% copies of the exogenous protein per cell,

contacting the cell population with 0.4±50%, ±20% or ±10% ug of mRNA per 5E6 cells in the population yields a population of cells expressing 870,000±50%, ±20% copies of the exogenous protein per cell,

contacting the cell population with 0.2±50%, ±20% or ±10% ug of mRNA per 5E6 cells in the population yields a population of cells expressing 610,000±50%, ±20%, or ±10% copies of the exogenous protein per cell,

contacting the cell population with 0.1±50%, ±20% or ±10% ug of mRNA per 5E6 cells in the population yields a population of cells expressing 270,000±50%, ±20%, or ±10% copies of the exogenous protein per cell,

contacting the cell population with 0.05±50%, ±20% or ±10% ug of mRNA per 5E6 cells in the population yields a population of cells expressing 100,000±50%, ±20%, or ±10% copies of the exogenous protein per cell, or

contacting the cell population with 0.025±50%, ±20% or ±10% ug of mRNA per 5E6 cells in the population yields a population of cells expressing 43,000±50%, ±20%, or ±10% copies of the exogenous protein per cell.

In embodiments, at least 50%, 60%, 70%, 80%, 85%, 90%, or 95% of the cells of the population express the exogenous protein 1 day after the cells are contacted with the exogenous protein.

In some embodiments of any of the aspects herein, the population of erythroid cells (e.g., the population of cells that is contacted with an mRNA) as described herein is a population of erythroid cells wherein one or more (e.g., 2, 3, 4, 5, 6, 7, 8 or more) of:

2-40%, 3-33%, 5-30%, 10-25%, or 15-20% of the cells in the population are enucleated;

less than 3%, 5%, 10%, 20%, or 30% of the cells in the population are enucleated;

greater than 0 (e.g., 0.1%, 0.2%, 0.5%) and no more than 50% (40%, 30%, 20%, 18%, 15%, 12%, 10%) of the cells in the population are enucleated;

the population of cells has reached 6-70%, 10-60%, 20-50%, or 30-40% of maximal enucleation;

the population of cells has reached less than 6%, 10%, 20%, 30%, 40%, 50%, or 60% of maximal enucleation;

the population of cells has a translational activity of at least 600,000, 800,000, 1,000,000, 1,200,000, 1,400,000, 1,600,000, 1,800,000, 2,000,000, 2,200,000, or 2,400,000 as measured by a BONCAT assay, e.g., by the translation assay of Example 10;

the population of cells has a translational activity of 600,000-2,400,000, 800,000-2,200,000, 1,000,000-2,000,000, 1,200,000-1,800,000, or 1,400,000-1,600,000 as measured by a BONCAT assay, e.g., by the translation assay of Example 10;

the population of cells in maturation phase has at least 10%, 20%, 30%, 40%, 50%, 60%, 70%, 80%, or 90% of maximal translational activity, wherein maximal translational activity refers to the maximal translational activity of a similar number of precursors or progenitors of the cells in maturation phase, e.g., CD34+ cells;

between 0.1-25% of the cells in the population are enucleated and the population of cells is fewer than 1, 2 or 3 population doublings from a plateau in cell division;

the population of cells is fewer than 3, 2, or 1 population doubling from a plateau in cell division;

the population of cells is capable of fewer than 3, 2, or 1 population doubling;

the population will increase by no more than 1.5, 2, or 3 fold before the population reaches an enucleation level of at least 70% of cells in the population;

84-99%, 85-95%, or about 90% of the cells in the population are GPA-positive (e.g., as measured by a flow cytometry assay, e.g., a flow cytometry assay of Example 10);

at least 84%, 85%, 90%, 95%, or 99% of the cells in the population are GPA-positive (e.g., as measured by a flow cytometry assay, e.g., a flow cytometry assay of Example 10);

54-99%, 55-98%, 60-95%, 65-90%, 70-85%, or 75-80% of the cells in the population are band3-positive (e.g., as measured by a flow cytometry assay, e.g., a flow cytometry assay of Example 10);

at least 54%, 55%, 60%, 65%, 70%, 75%, 80%, 85%, 90%, 95%, 98%, or 99% of the cells in the population are band3-positive (e.g., as measured by a flow cytometry assay, e.g., a flow cytometry assay of Example 10);

96-100%, 97-99%, or about 98% of the cells in the population are alpha4 integrin-positive (e.g., as measured by a flow cytometry assay, e.g., a flow cytometry assay of Example 10);

at least 95%, 96%, 97%, 98%, or 99% of the cells in the population are alpha4 integrin-positive (e.g., as measured by a flow cytometry assay, e.g., a flow cytometry assay of Example 10);

at least 50% (e.g., at least 60%, 70%, 80%, 85%, 90%, 92%, 94%, 96%) of the cells in the population are alpha4 integrin-positive and band3-positive; or

at least 50% of the cells in the population are band3-positive and at least 90%-95% are alpha4 integrin-positive.

The disclosure contemplates all combinations of any one or more of the foregoing aspects and/or embodiments, as well as combinations with any one or more of the embodiments set forth in the detailed description and examples.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jul. 7, 2016. When one gene or protein references a plurality of sequence accession numbers, all of the sequence variants are encompassed.

### BRIEF DESCRIPTION OF THE DRAWINGS

FIG. 1 is a plot showing expression of various constructs of differing sizes on K562 erythroleukemia cells following lentiviral transduction. Each data point represents a unique construct. Expression is measured by flow cytometry with an anti-HA antibody, as every construct contains the appropriate epitope tag. The constructs are arrayed by provirus length, which is the length of nucleic acid in the viral genome (including the transgene itself) that will be integrated into the target cell genome.

FIG. 2 is a plot showing a characterization of lentivirus particles that contain transgenes of various lengths such that the provirus ranges from approximately 3.5 kb to approximately 8.5 kb. The y-axis shows RNA copies per ug of p24. The number of RNA copies per mL of viral supernatant is measured by qPCR. The amount of p24 (ug) per mL of viral supernatant is measured by ELISA against p24. The ratio of the two measured values gives the number of RNA copies per mass p24.

FIG. 3 shows flow cytometry histograms showing the expression of GFP in K562 cells and erythroid cells cultured from primary progenitors as measured by flow cytometry 24 hrs following electroporation of cells with GFP mRNA using conditions optimized for K562 cells.

FIG. 4A, FIG. 4B, and FIG. 4C are flow cytometry histograms showing the expression of GFP in erythroid cells cultured from primary progenitors as measured by flow cytometry 24 hrs following electroporation of cells with GFP mRNA. 12 different conditions are shown (numbers 1-12). In the first column, GFP fluorescence is detected. In the second column, cell viability is measured with Life Technologies LIVE/DEAD stain, wherein the dead cells are stained by the dye, such that the percentage of live cells is 100%−% Fluorescent Cells.

FIG. 5 shows flow cytometry histograms showing expression of GFP in erythroid cells cultured from primary progenitors at various stages of differentiation as measured by flow cytometry 24 hrs following electroporation of cells with GFP mRNA. Untransfected cells are compared to GFP mRNA transfected cells. The columns refer to the number of days of erythroid differentiation prior to transfection. The percent viability is measured with Life Technologies LIVE/DEAD stain and is reported as the % of viable cells, that is, cells that stain negative for the dye.

FIG. 6 shows flow cytometry histograms showing the expression of GFP in erythroid cells cultured from primary progenitors as measured by flow cytometry 24 hrs following electroporation of cells with GFP mRNA. Cells were transfected at day 9 of culture then returned to differentiation media and re-analyzed at day 13. At day 13, cells were re-electroporated with GFP mRNA and analyzed for expression 24 hrs later.

FIGS. 7A, 7B, and 7C show the percent of GFP positive erythroid cells electroporated at different timepoints after the start of in vitro differentiation. FIG. 7A illustrates the expansion, differentiation, and maturation phases. FIG. 7B shows the percentage of GFP positive cells after electroporation on differentiation day 9, when assayed through maturation day 9. FIG. 7C shows the percentage of GFP positive cells after electroporation on maturation day 7, when assayed through maturation day 16. “No EP” denotes the no-electroporation control. “P1-P4” denote four electroporation conditions.

FIGS. 8A and 8B are graph showing GFP expression in erythroid cells expressing GFP at the indicated timepoints, when the erythroid cells were electroporated with mRNA encoding GFP on days M4 through M7 of maturation. FIG. 8A shows the percentage of cells expressing GFP, and FIG. 8B shows the mean fluorescent intensity of the cells.

FIG. 9 is a graph showing a time course of erythroid cell maturation. Circles indicate levels of translation, measured by AHA intensity/incorporation. Squares indicate enucleation levels.

FIG. 10 is a graph showing a time course of erythroid cell maturation, where the percentage of cells expressing mCherry is shown on the y-axis. EP, electroporated control (without RNasin). UT no EP, untransfected control, no electroporation. EP+RNasin 0.5, electroporated sample treated with 0.5 U/uL RNasin. EP+RNasin 1, electroporated sample treated with 1 U/uL RNasin. EP+RNasin 2, electroporated sample treated with 2 U/uL RNasin.

FIG. 11 is a graph showing effective expression (mean fluorescent intensity×number of fluorescent cells)/1×10⁶) versus time of cells treated with proteasome inhibitors at different timepoints.

FIG. 12 is a graph showing percentage of GFP-positive cells for cells electrporated with GFP-PAL naked mRNA, GFP-PAL polyA Cap mRNA, GFP-PAL naked modified mRNA, or GFP-PAL polyA Cap modified mRNA at day M4. GFP expression was measured by flow cytometry at days M5 (24 hours later), M6, M7, and M10.

### DETAILED DESCRIPTION OF THE INVENTION

### Definitions

As used herein, the term “antibody molecule” refers to a protein, e.g., an immunoglobulin chain or fragment thereof, comprising at least one immunoglobulin variable domain sequence. The term “antibody molecule” encompasses antibodies and antibody fragments. In an embodiment, an antibody molecule is a multispecific antibody molecule, e.g., a bispecific antibody molecule. Examples of antibody molecules include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

As used herein, “differentiating conditions” are conditions under which an erythroid precursor cell, e.g., an HSC, or CD34+ cell, is amplified and differentiated into an enucleated erythroid cell (e.g., an enucleated reticulocyte or erythrocyte) in ex-vivo culture, typically with the addition of erythropoietin and other growth factors. This process typically includes a proliferation/expansion phase, a differentiation phase, and a maturation phase (during which the cells lose their nuclei). Differentiating conditions are known in the art. See for example, Olivier et al., Novel, High-Yield Red Blood Cell Production Methods from CD34-Positive Cells Derived from Human Embryonic Stem, Yolk Sac, Fetal Liver, Cord Blood, and Peripheral Blood. Stem Cells Transl Med. 2012 August; 1(8): 604-614, and references cited therein. “Erythroid cells” as used herein are cells of the erythrocytic series including erythroid precursor cells such as hematopoietic stem cells (HSCs) and nucleated erythroid precursor cells such as CD34+ cells, nucleated red blood cell precursors, enucleated red blood cells (e.g., reticulocytes or erythrocytes), and any intermediates between erythroid precursor cells and enucleated erythrocytes. In an embodiment, an erythroid cell is a proerythroblast, basophilic erythroblast, polychromatophilic erythroblast, orthochromatic erythroblast, reticulocyte, or erythrocyte. In an embodiment, an erythroid cell is a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, or a combination thereof.

In embodiments, the erythroid cells are, or are derived from, immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al. (2013) Mol Ther, epub ahead of print September 3). In addition, the cells may be intended for autologous use or provide a source for allogeneic transfusion. In some embodiments, erythroid cells are cultured.

As used herein, “enucleated” refers to a cell that lacks a nucleus, e.g., a cell that lost its nucleus through differentiation into a mature red blood cell.

“Exogenous polypeptide” refers to a polypeptide that is not produced by a wild-type cell of that type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell.

“Exogenous” when used to modify the term mRNA, refers to the relationship between the mRNA and a selected subject cell, e.g., an erythroid cell, e.g., an enucleated erythroid cell. An exogenous mRNA does not exist naturally in the subject cell. In an embodiment an exogenous mRNA expresses a polypeptide that does not occur naturally in the selected subject cell (an exogenous polypeptide). In embodiments an exogenous mRNA comprises a first portion that does not occur naturally in the selected subject cell and a second portion that does occur naturally in the selected subject cell.

“Heterologous” when used to modify the term untranslated region (UTR), refers to the relationship between the UTR and a coding region with which the UTR is operatively linked (the subject coding region). A UTR is a heterologous UTR if it has one or more of the following properties: i) it does not exist in nature; ii) it does not occur naturally with the subject coding region, e.g., differs by at least 1 nucleotide, e.g., by at least 1, 2, 3, 4, 5, 10, 20, 25, 30, 35, 40, 45, or 50% of its nucleotides, from the UTR which occurs naturally operatively linked with the subject coding region; or iii) wherein the UTR does not occur naturally operatively linked to the subject coding region but occurs naturally operatively linked with a coding region other than the subject coding region, or has at least at least 70, 80, 90, 95, 99, or 100% homology to such naturally occurring UTR.

“Modified” as used herein in reference to a nucleic acid, refers to a structural characteristic of that nucleic acid that differs from a canonical nucleic acid. It does not imply any particular process of making the nucleic acid or nucleotide.

The term “regulatory element”, as used herein in reference to an RNA sequence, refers to a sequence that is capable of modulating (e.g., upregulating or downregulating) a property of the RNA (e.g., stability or translatability, e.g., translation level of the coding region to which the regulatory element is operatively linked) in response to the presence or level of a molecule, e.g., a small molecule, RNA binding protein, or regulatory RNA such as a miRNA.

### Chemically Modified Nucleic Acids

The exogenous RNA can comprise unmodified or modified nucleobases. Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27: 196-197). An RNA can also comprise wholly synthetic nucleotides that do not occur in nature.

In some embodiments, the chemically modification is one provided in PCT/US2016/032454, US Pat. Pub. No. 20090286852, of International Application No. WO/2012/019168, WO/2012/045075, WO/2012/135805, WO/2012/158736, WO/2013/039857, WO/2013/039861, WO/2013/052523, WO/2013/090648, WO/2013/096709, WO/2013/101690, WO/2013/106496, WO/2013/130161, WO/2013/151669, WO/2013/151736, WO/2013/151672, WO/2013/151664, WO/2013/151665, WO/2013/151668, WO/2013/151671, WO/2013/151667, WO/2013/151670, WO/2013/151666, WO/2013/151663, WO/2014/028429, WO/2014/081507, WO/2014/093924, WO/2014/093574, WO/2014/113089, WO/2014/144711, WO/2014/144767, WO/2014/144039, WO/2014/152540, WO/2014/152030, WO/2014/152031, WO/2014/152027, WO/2014/152211, WO/2014/158795, WO/2014/159813, WO/2014/164253, WO/2015/006747, WO/2015/034928, WO/2015/034925, WO/2015/038892, WO/2015/048744, WO/2015/051214, WO/2015/051173, WO/2015/051169, WO/2015/058069, WO/2015/085318, WO/2015/089511, WO/2015/105926, WO/2015/164674, WO/2015/196130, WO/2015/196128, WO/2015/196118, WO/2016/011226, WO/2016/011222, WO/2016/011306, WO/2016/014846, WO/2016/022914, WO/2016/036902, WO/2016/077125, WO/2016/077123, each of which is herein incorporated by reference in its entirety. It is understood that incorporation of a chemically modified nucleotide into a polynucleotide can result in the modification being incorporated into a nucleobase, the backbone, or both, depending on the location of the modification in the nucleotide. In some embodiments, the backbone modification is one provided in EP 2813570, which is herein incorporated by reference in its entirety. In some embodiments, the modified cap is one provided in US Pat. Pub. No. 20050287539, which is herein incorporated by reference in its entirety.

In some embodiments, the modified mRNA comprises one or more of ARCA: anti-reverse cap analog (m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), and Ψ (pseudouridine triphosphate). In embodiments, the modified mRNA comprises N6-methyladenosine. In embodiments, the modified mRNA comprises pseudouridine.

In some embodiments, the exogenous RNA comprises a backbone modification, e.g., a modification to a sugar or phosphate group in the backbone. In some embodiments, the exogenous RNA comprises a nucleobase modification.

In some embodiments, the exogenous mRNA comprises one or more chemically modified nucleotides of Table 1, one or more chemical backbone modifications of Table 2, one or more chemically modified caps of Table 3. For instance, in some embodiments, the exogenous mRNA comprises two or more (e.g., 3, 4, 5, 6, 7, 8, 9, or 10 or more) different types of chemical modifications. As an example, the exogenous mRNA may comprise two or more (e.g., 3, 4, 5, 6, 7, 8, 9, or 10 or more) different types of modified nucleobases, e.g., as described herein, e.g., in Table 1. Alternatively or in combination, the exogenous mRNA may comprise two or more (e.g., 3, 4, 5, 6, 7, 8, 9, or 10 or more) different types of backbone modifications, e.g., as described herein, e.g., in Table 2. Alternatively or in combination, the exogenous mRNA may comprise two or more (e.g., 3, 4, 5, 6, 7, 8, 9, or 10 or more) different types of modified cap, e.g., as described herein, e.g., in Table 3. For instance, in some embodiments, the exogenous mRNA comprises one or more type of modified nucleobase and one or more type of backbone modification; one or more type of modified nucleobase and one or more modified cap; one or more type of modified cap and one or more type of backbone modification; or one or more type of modified nucleobase, one or more type of backbone modification, and one or more type of modified cap.

In some embodiments, the exogenous mRNA comprises one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) modified nucleobases. In some embodiments, all nucleobases of the mRNA are modified. In some embodiments, the exogenous mRNA is modified at one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) positions in the backbone. In some embodiments, all backbone positions of the mRNA are modified.

### Heterologous Untranslated Regions

The exogenous mRNAs described herein can comprise one or more (e.g., two, three, four, or more) heterologous UTRs. The UTR may be, e.g., a 3′ UTR or 5′ UTR. In embodiments, the heterologous UTR comprises a eukaryotic, e.g., animal, e.g., mammalian, e.g., human UTR sequence, or a portion or variant of any of the foregoing. In embodiments, the heterologous UTR comprises a synthetic sequence. In embodiments, the heterologous UTR is other than a viral UTR, e.g., other than a hepatitis virus UTR, e.g., other than Woodchuck hepatitis virus UTR.

While not wishing to be bound by theory, in some embodiments, the 5′ UTR is short, in order to reduce scanning time of the ribosome during translation. In embodiments, the untranslated region is less than 1000, 900, 800, 700, 600, 500, 400, 300, 200, 100, 50, 40, 30, 20, 10, or 5 nucleotides in length. In embodiments, the 5′UTR comprises a sequence having not more than 1000, 900, 800, 700, 600, 500, 400, 300, 200, 100, 50, 40, 30, 20, 10, or 5 consecutive nucleotides from a naturally occurring 5′ UTR. In embodiments, the RNA lacks a 5′ UTR.

In some embodiments, the 5′ UTR does not comprise an AUG upstream of the start codon (uAUG). According to the non-limiting theory herein, some naturally occurring 5′ UTRs contain one or more uAUGs which can regulate, e.g., reduce, translation of the encoded gene. Sometimes, the uAUGs are paired with stop codons, to form uORFs. Accordingly, in some embodiments, the 5′ UTR has sequence similarity to a naturally occurring 5′ UTR, but lacks one or more uAUGs or uORFs relative to the naturally occurring 5′ UTR. The one or more uAUGs can be removed, e.g., by a deletion or substitution mutation.

It is understood that the heterologous UTRs provided herein can be provided as part of a purified RNA, e.g., by contacting an erythroid cell with an mRNA comprising the heterologous UTR. The heterologous UTRs herein can also be provided via DNA, e.g., by contacting the erythroid cell with DNA under conditions that allow the cell to transcribe the DNA into an RNA that comprises the heterologous UTR.

In embodiments, the 3′ UTR comprises a polyA tail, e.g., at least 10, 20, 30, 40, 50, 100, 150, 200, 250, 300, 350, 400, 450, 500, 600, 700, 800, 900, or 1000 adenosines.

In embodiments, the exogenous RNA comprises a 5′ UTR and 3′ UTR that allow circularization of the RNA through binding of an upstream element to a downstream element, directly or indirectly. In embodiments, the exogenous RNA comprises a 5′ cap that participates in circularization.

UTRs Comprising Regulatory Elements

In embodiments, the UTR comprises a regulatory element. The regulatory element may modulate (e.g., upregulate or downregulate) a property (e.g., stability or translation level) of the coding region to which it is operatively linked. In some embodiments, the regulatory element controls the timing of translation of the RNA. For instance, the RNA may be translated in response to phase of the cell cycle, presence or level of a pathogen (e.g., a virus that enters the cell), stage of red blood cell differentiation, presence or level of a molecule inside the cell (e.g., a metabolite, a signalling molecule, or an RNA such as a miRNA), or presence or level of a molecule outside the cell (e.g., a protein that is bound by a receptor on the surface of the red blood cell).

In embodiments, the regulatory element comprises a riboregulator, e.g., as described in Callura et al., “Tracking, tuning, and terminating microbial physiology using synthetic riboregulators” PNAS 107:36, p. 15898-15903. In embodiments, the riboregulator comprises a hairpin that masks a ribosome binding site, thus repressing translation of the mRNA. In embodiments, a trans-activating RNA binds to and opens the hairpin, exposing the ribosome binding site, and allowing the mRNA to be translated. In embodiments, the ribosome binding site is an IRES, e.g., a Human IGF-II 5′ UTR-derived IRES described in Pedersen, S K, et al., Biochem J. 2002 Apr. 1; 363(Pt 1): 37-44:

GACCGGG CATTGCCCCC AGTCTCCCCC AAATTTGGGC ATTGTCCCCG GGTCTTCCAA CGGACTGGGC GTTGCTCCCG GACACTGAGG ACTGGCCCCG GGGTCTCGCT CACCTTCAGC AG (SEQ ID NO: 2)

In embodiments, the regulatory element comprises a toehold switch, e.g., as described in International Application WO2012058488. In embodiments, the toehold functions like a riboregulator and further comprises a short single stranded sequence called a toehold, which has homology to a trans-regulating RNA. In embodiments, the toehold can sample different binding partners, thereby more rapidly detecting whether the trans-regulating RNA is present.

In embodiments, the regulatory element is one described in Araujo et al., “Before It Gets Started: Regulating Translation at the 5′ UTR” Comparative and Functional Genomics, Volume 2012 (2012), Article ID 475731, 8 pages, which is herein incorporated by reference in its entirety.

In embodiments, the regulatory element comprises an upstream open reading frame (uORF). A uORF comprises a uAUG and a stop codon in-frame with the uAUG. uORFs often act as negative regulators of translation, when a ribosome translates the uORF and then stalls at the stop codon, without reaching the downstream coding region. An exemplary uORFs is that found in the fungal arginine attenuator peptide (AAP), which is regulated by arginine concentration. Another exemplary uORF is found in the yeast GCN4, where translation is activated under amino acid starvation conditions. Another uORF is found in Carnitine Palmitoyltransferase 1C (CPT1C) mRNA, where repression is relieved in response to glucose deprivation. In some embodiments, the uORF is a synthetic uORF. In some embodiments, the uORF is one found in the 5′ UTR of the mRNA for cyclin-dependent-kinase inhibitor protein (CDKN2A), thrombopoietin, hairless homolog, TGF-beta3, SRY, IRF6, PRKAR1A, SPINK1, or HBB.

In embodiments, the regulatory element comprises a secondary structure, such as a hairpin. In embodiments, the hairpin has a free energy of about −30, −40, −50, −60, −70, −80, −90, or −100 kcal/mol or stronger and is sufficient to reduce translation of the mRNA compared to an mRNA lacking the hairpin. In embodiments, the secondary structure is one found in TGF-beta1 mRNA, or a fragment or variant thereof, that binds YB-1.

In embodiments, the regulatory element comprises an RPB (RNA-binding protein) biding motif. In embodiments, the RNA binding protein comprises HuR, Musashi, an IRP (e.g., IRP1 or IRP2), SXL, or lin-14. In embodiments, the regulatory element comprises an IRE, SXL binding motif, p21 5′ UTR GC-rich stem loop, or lin-4 motif. IRP1 and IRP2 bind to a stem-loop sequence called an iron-response element (IRE); binding creates a steric block to translation. The SXL protein binds a SXL binding motif, e.g., a poly-U stretches in an intron in the 5′ UTR, causing intron retention. The SXL protein also binds a poly-U region in the 3′ UTR, to block recruitment of the pre-initiation complex and repress translation. SXL also promotes translation of a uORF, repressing translation of the main coding region. The p21 5′ UTR GC-rich stem loop is bound by CUGBP1 (a translational activator) or calreticulin (CRT, a translational repressor).

In some embodiments, the regulatory element comprises a binding site for a trans-acting RNA. In some embodiments, the trans-acting RNA is a miRNA. In embodiments, the untranslated region comprises an RNA-binding sequence, e.g., the lin-14 3′ UTR which comprises conserved sequences that are bound by lin-4 RNA, thereby down-regulating translation of the lin-14 RNA (Wightman et al., Cell, Vol. 75, 855-862, Dec. 3, 1993).

In embodiments, the regulatory element comprises a sequence that binds ribosomal RNA, e.g., that promotes shunting of the ribosome to bypass a segment of the 5′ UTR and arrive at the start codon. In embodiments, the regulatory sequence that promotes shunting is a sequence found in cauliflower mosaic virus or adenovirus.

UTRs of Red Blood Cell Proteins

In some embodiments, the untranslated region is a UTR of an RNA that is expressed in a wild-type erythroid cell, e.g., in a mature red blood cell. In embodiments, the UTR is a UTR of a gene for a type I red blood cell transmembrane protein (e.g., glycophorin A), a type II red blood cell transmembrane protein (e.g., Kell or CD71), or a type III red blood cell transmembrane protein such as GLUT1. In embodiments, the UTR is a UTR of a red blood cell protein such as CD235a, c-Kit, GPA, IL3R, CD34, CD36, CD71, Band 3, hemoglobin, and Alpha 4 integrin. In embodiments, the UTR is a UTR of a gene for spectrin, ankyrin, 4.1R, 4.2, p55, tropomodulin, or 4.9.

In some embodiments, the untranslated region comprises a hemoglobin UTR, e.g., the 3′ hemoglobin UTR of SEQ ID NO: 1: GCTCGCTTTCTTGCTGTCCAATTTCTATTAAAGGTTCCTTTGTTCCCTAAGTCCAACT ACTAAACTGGGGGATATTATGAAGGGCCTTGAGCATCTGGATTCTGCCTAATAAAAA ACATTTATTTTCATTGC. In embodiments, the untranslated region comprises a stretch of at least 10, 20, 30, 40, 50, 60, 70, 80, 90, 100, 110, 120, or 130 nucleotides of SEQ ID NO: 1. In embodiments, the untranslated region comprises a sequence with at least 70%, 75%, 80%, 85%, 90%, 95%, 96%, 97%, 98%, or 99% identity to the sequence of SEQ ID NO: 1.

In some embodiments, the exogenous mRNA comprises a heterologous 3′ UTR. In some embodiments, the exogenous mRNA comprises a heterologous 5′ UTR. In some embodiments, the exogenous mRNA comprises a heterologous 3′ UTR and a heterologous 5′ UTR.

### Regulatory RNAs

The invention includes, in some aspects, an erythroid cell comprising a regulatory RNA. In some embodiments, the cell further comprises an exogenous mRNA.

In related aspects, the invention includes a method of contacting an erythroid cell with a regulatory RNA. In embodiments, the method further comprises contacting the cell with an exogenous mRNA. In embodiments, the cell is contacted with the exogenous mRNA before, during, or after the contacting with the regulatory RNA.

In related aspects, the invention includes a composition (e.g., a purified or isolated composition) comprising: (i) a regulatory RNA (e.g., a miRNA or an anti-miR), and (ii) an exogenous mRNA described herein, e.g., an mRNA that is codon-optimized for expression in a human cell (e.g., in a human erythroid cell), an mRNA comprising a red blood cell transmembrane segment, or an mRNA comprising a heterologous UTR described herein (such as a hemoglobin UTR or a UTR from another red blood cell protein).

In embodiments, the regulatory RNA modulates a property (e.g., stability or translation) of the exogenous mRNA. In some embodiments, the regulatory RNA affects the erythroid cell, e.g., affects its proliferation or differentiation. In some embodiments, affecting proliferation comprises increasing the number of divisions a starting cell makes (e.g., in culture) and/or increasing the total number of cells produced from a starting cell or population. In some embodiments, regulating differentiation comprises promoting maturation and/or enucleation. In some embodiments, the regulatory RNA encodes EPO and, e.g., stimulates expansion of erythroid cells.

In embodiments, the regulatory RNA is a miRNA. In some embodiments, the miRNA is a human miRNA, e.g., an miRNA listed in Table 12 herein, e.g., one of the elements of Table 12 with a designation beginning with “MIR”, or a sequence with no more than 1, 2, 3, 4, or 5 alterations (e.g., substitutions, insertions, or deletions) relative thereto.

In some embodiments, the regulatory RNA is an anti-miR. In some embodiments, an anti-miR inhibits a miRNA (such as an endogenous miRNA) by hybridizing with the miRNA and preventing the miRNA from binding its target mRNA. In some embodiments, the anti-miR binds and/or has complementarity to a human miRNA, e.g., an miRNA listed in Table 12 herein, e.g., one of the elements of Table 12 with a designation beginning with “MIR”, or a sequence with no more than 1, 2, 3, 4, or 5 alterations (e.g., substitutions, insertions, or deletions) relative thereto.

In some embodiments, the regulatory RNA is a siRNA, shRNA, or antisense molecule. In embodiments, the siRNA comprises a sense strand and an antisense strand which can hybridize to each other, wherein the antisense strand can further hybridize to a target mRNA; may have one or two blunt ends; may have one or two overhangs such as 3′ dTdT overhangs; may comprise chemical modifications; may comprise a cap; and may comprise a conjugate. In embodiments, the shRNA comprises a hairpin structure with a sense region, an antisense region, and a loop region, wherein the sense region and antisense region can hybridize to each other, wherein the antisense region can further hybridize to a target mRNA; may have a blunt end; may have an overhang; may comprise chemical modifications; may comprise a cap; and may comprise a conjugate. In embodiments, the antisense molecule comprises a single strand that can hybridize to a target mRNA; may comprise chemical modifications; may comprise a cap; and may comprise a conjugate.

### Lipid Nanoparticle Methods

In some embodiments, an RNA (e.g., mRNA) described herein is introduced into an erythroid cell using lipid nanoparticle (LNPs), e.g., by transfection. Thus, in some aspects, the disclosure provides a method of introducing an mRNA encoding an exogenous protein into an erythroid cell, comprising contacting the erythroid cell with the mRNA and an LNP, e.g., an LNP described herein. The disclosure also provides reaction mixtures comprising an erythroid cell, an mRNA, and an LNP. In some embodiments, the mRNA is complexed with the LNP. In embodiments, the population of cells contacted with the LNPs comprises at least 1×10⁷, 2×10⁷, 5×10⁷, 1×10⁸, 2×10⁸, 5×10⁸, 1×10⁹, 2×10⁹, or 5×10⁹, 1×10¹⁰, 2×10¹⁰, or 5×10¹⁰ cells.

An exemplary LNP comprises a cationic trialkyl lipid, a non-cationic lipid (e.g., PEG-lipid conjugate and a phospholipid), and an mRNA molecule that is encapsulated within the lipid particle. In embodiments, the phospholipid comprises dipalmitoylphosphatidylcholine (DPPC), distearoylphosphatidylcholine (DSPC), or a mixture thereof. In embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof. In embodiments, the PEG-DAA conjugate is selected from the group consisting of a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof. In embodiments, the LNP further comprises cholesterol. Additional LNPs are described, e.g., in US Pat. Pub. 20160256567, which is herein incorporated by reference in its entirety.

Another exemplary LNP can comprise a lipid having a structural Formula (I):

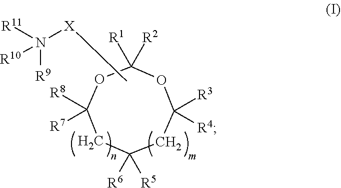

or salts thereof, wherein:
R¹, R², R³, R⁴, R⁵, R⁶, R⁷, and R⁸ are independently selected from the group consisting of hydrogen, optionally substituted C₇-C₃₀ alkyl, optionally substituted C₇-C₃₀ alkenyl and optionally substituted C₇-C₃₀ alkynyl;
provided that (a) at least two of R¹, R², R³, R⁴, R⁵, R⁶, R⁷, and R⁸ are not hydrogen, and (b) two of the at least two of R¹, R², R³, R⁴, R⁵, R⁶, R⁷, and R⁸ that are not hydrogen are present in a 1, 3 arrangement, a 1, 4 arrangement or a 1, 5 arrangement with respect to each other;
X is selected from the group consisting of C₁-C₆ alkyl, C₂-C₆ alkenyl and C₂-C₆ alkynyl;
R⁹, R¹⁰, and R¹¹ are independently selected from the group consisting of hydrogen, optionally substituted C₁-C₇ alkyl, optionally substituted C₂-C₇ alkenyl and optionally substituted C₂-C₇ alkynyl, provided that one of R⁹, R¹⁰, and R¹¹ may be absent; and
n and m are each independently 0 or 1.
For instance, the lipid can comprise one of the following structures:

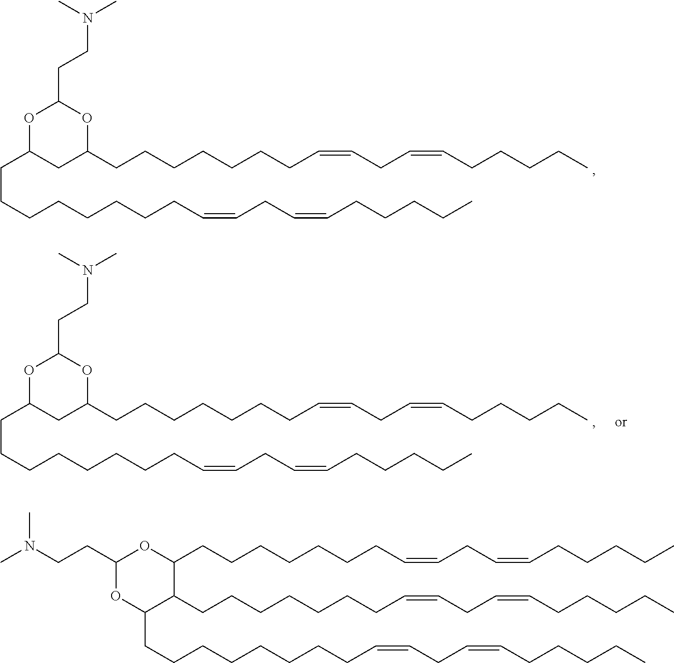

In embodiments, the LNP further comprises a non-cationic lipid such as a phospholipid, cholesterol, or a mixture of a phospholipid and cholesterol. In embodiments, the phospholipid comprises dipalmitoylphosphatidylcholine (DPPC), distearoylphosphatidylcholine (DSPC), or a mixture thereof. Additional LNPs are described, e.g., in US Pat. Pub. 20130064894, which is herein incorporated by reference in its entirety.

Another exemplary LNP comprises: (a) a nucleic acid, e.g., mRNA; (b) a cationic lipid comprising from 50 mol % to 65 mol % (e.g., 52 mol % to 62 mol %) of the total lipid present in the particle; (c) a non-cationic lipid comprising a mixture of a phospholipid and cholesterol or a derivative thereof, wherein the phospholipid comprises from 4 mol % to 10 mol % of the total lipid present in the particle and the cholesterol or derivative thereof comprises from 30 mol % to 40 mol % of the total lipid present in the particle; and (d) a conjugated lipid that inhibits aggregation of particles comprising from 0.5 mol % to 2 mol % of the total lipid present in the particle. In embodiments, the phospholipid comprises dipalmitoylphosphatidylcholine (DPPC), distearoylphosphatidylcholine (DSPC), or a mixture thereof. In embodiments, the conjugated lipid that inhibits aggregation of particles comprises a polyethyleneglycol (PEG)-lipid conjugate. In embodiments, the PEG-lipid conjugate comprises a PEG-diacylglycerol (PEG-DAG) conjugate, a PEG-dialkyloxypropyl (PEG-DAA) conjugate, or a mixture thereof. In embodiments, the PEG-DAA conjugate comprises a PEG-dimyristyloxypropyl (PEG-DMA) conjugate, a PEG-distearyloxypropyl (PEG-DSA) conjugate, or a mixture thereof. Additional LNPs are described, e.g., in U.S. Pat. No. 8,058,069, which is herein incorporated by reference in its entirety.

### Methods of Manufacturing Erythroid Cells

Methods of differentiating erythroid precursor cells into mature erythroid cells are known. See, for example, Douay & Andreu. Transfus Med Rev. 2007 April; 21(2):91-100; Giarratana et al. Nat Biotechnol. 2005 January; 23(1):69-74; Olivier et al. Stem Cells Transl Med. 2012 August; 1(8): 604-614, and references cited therein.

Methods of manufacturing erythroid cells comprising (e.g., expressing) exogenous RNAs and/or proteins are described, e.g., in WO2015/073587 and WO2015/153102, each of which is incorporated by reference in its entirety.

In some embodiments, hematopoietic progenitor cells, e.g., CD34+ hematopoietic progenitor cells, are contacted with a nucleic acid or nucleic acids encoding one or more exogenous polypeptides, and the cells are allowed to expand and differentiate in culture.

In embodiments, the method comprises a step of electroporating the cells, e.g., as described herein.

In some embodiments, the erythroid cells are expanded at least 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated cells. In some embodiments, the population of erythroid cells contains less than 1% live enucleated cells, e.g., contains no detectable live enucleated cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise an exogenous RNA and/or polypeptide. Expression of an exogenous polypeptide is measured, in some embodiments, by FACS using labeled antibodies against the polypeptide. In some embodiments, the population of enucleated cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹²-1×10¹², 1×10¹²-2×10¹², 2×10¹²-5×10¹², or 5×10¹²-1×10¹³ cells.

### Exemplary Exogenous Polypeptides and Uses Thereof

One or more of the exogenous proteins may have post-translational modifications characteristic of eukaryotic cells, e.g., mammalian cells, e.g., human cells. In some embodiments, one or more (e.g., 2, 3, 4, 5, or more) of the exogenous proteins are glycosylated, phosphorylated, or both. In vitro detection of glycoproteins is routinely accomplished on SDS-PAGE gels and Western Blots using a modification of Periodic acid-Schiff (PAS) methods. Cellular localization of glycoproteins may be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments, one or more of the exogenous polypeptides is a fusion protein, e.g., is a fusion with an endogenous red blood cell protein or fragment thereof, e.g., a transmembrane protein, e.g., GPA or a transmembrane fragment thereof.

In some embodiments, the coding region for the exogenous polypeptide is codon-optimized for the cell in which it is expressed, e.g., a mammalian erythroid cell, e.g., a human erythroid cell.

In some embodiments, the erythroid cells comprise at least 1,000, 2,000, 5,000, 10,000, 20,000, 50,000, or 100,000 copies of the exogenous protein per cell. In embodiments, the copy number of the exogenous protein can be determined, e.g., by quantitative Western blot or using standardized fluorescent microspheres (e.g., from Bangs Laboratories) in a flow cytometry assay. In some embodiments, e.g., wherein the exogenous protein is a fluorescent protein, the mean fluorescent intensity (MFI) can be used to estimate protein copy number, e.g., by determining the MFI of a sample, quantifying the copy number of the fluorescent protein in a similar sample (e.g., by quantitative Western blot), and calculating a conversion factor between MFI and protein copy number.

### Physical Characteristics of Enucleated Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell (e.g., an erythroid cell not subjected to hypotonic dialysis). In contrast, a hypotonically loaded RBC sometimes displays altered physical characteristics such as increased osmotic fragility, altered cell size, reduced hemoglobin concentration, or increased phosphatidylserine levels on the outer leaflet of the cell membrane.

Osmotic Fragility

In some embodiments, the enucleated erythroid cell exhibits substantially the same osmotic membrane fragility as an isolated, uncultured erythroid cell that does not comprise an exogenous polypeptide. In some embodiments, the population of enucleated erythroid cells have an osmotic fragility of less than 50% cell lysis at 0.3%, 0.35%, 0.4%, 0.45%, or 0.5% NaCl. In some embodiments, the population of enucleated erythroid cells has an osmotic fragility of less than 50% cell lysis in a solution consisting of 0.3%, 0.35%, 0.4%, 0.45%, or 0.5% NaCl in water. Osmotic fragility is determined, in some embodiments, using the method of Example 59 of WO2015/073587.

Cell Size

In some embodiments, the enucleated erythroid cell has approximately the diameter or volume as a wild-type, untreated reticulocyte. In some embodiments, the enucleated erythroid cell has a volume of about 150 fL, e.g., about 140-160, 130-170, or 120-180 fL. In some embodiments, the population has a mean cell volume of about 150 fL, about 140-160, 130-170, 120-180, 110-190, or 100-200 fL. In some embodiments, at least about 50%, 60%, 70%, 80%, 90%, 95%, or 99% of the cells in the population have a volume of between about 140-160, 130-170, 120-180, 110-190, or 100-200 fL. In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. Volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell, e.g., a mature RBC. In some embodiments, the erythroid cell comprises greater than 1%, 2%, 3%, 4%, 5%, 6%, 7%, 8%, 9% or greater than 10% fetal hemoglobin. In some embodiments, the erythroid cell comprises at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587.

Phosphatidylserine Content

In some embodiments, the enucleated erythroid cell has approximately the same phosphatidylserine content on the outer leaflet of its cell membrane as a wild-type, untreated RBC. Phosphatidylserine is predominantly on the inner leaflet of the cell membrane of wild-type, untreated RBCs, and hypotonic loading can cause the phosphatidylserine to distribute to the outer leaflet where it can trigger an immune response. In some embodiments, the population of RBC comprises less than about 30, 25, 20, 15, 10, 9, 8, 6, 5, 4, 3, 2, or 1% of cells that are positive for Annexin V staining. In some embodiments, at least 50%, 55%, 60%, 65%, 70%, 75%, 80%, 85%, 90%, 91%, 92%, 93%, 94%, 95%, 96%, 97%, 98%, or 99% of the enucleated cells in the population have the same as level of phosphatidylserine exposure as an otherwise similar cultured erythroid cell that does not express an exogenous protein. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587.

Other Characteristics

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the erythroid cells have a half-life of at least 30, 45, or 90 days in a subject.

Phases of Erythroid Cell Differentiation and Maturation

In embodiments, enucleated erythroid cells are produced by exposing CD34+ stem cells to three conditions: first expansion, then differentiation, and finally maturation conditions. Exemplary expansion, differentiation, and maturation conditions are described, e.g., as steps 1, 2, and 3 respectively in Example 3, paragraph [1221] of WO2015/073587, which is herein incorporated by reference in its entirety. In embodiments, expansion phase comprises culturing the cells in an expansion medium (e.g., medium of step 1 above), differentiation phase comprises culturing the cells in a differentiation medium (e.g., medium of step 2 above), and maturation phase comprises culturing the cells in a maturation medium (e.g., medium of step 3 above).

In embodiments, maturation phase begins when about 84% of the cells in the population are positive for GPA, e.g., as measured by a flow cytometry assay. In embodiments, at the beginning of maturation phase, a population of cells is about 54% band3-positive, e.g., as measured by a flow cytometry assay. In embodiments, at the beginning of maturation phase, a population of cells is about 98% alpha4 integrin-positive, e.g., as measured by a flow cytometry assay. In an embodiment, maturation phase begins when about 53% of cells in the erythroid cell population are positive for both band3 and alpha4 integrin. In embodiments, maturation phase begins when the cell population is predominantly pre-erythroblasts and basophilic erythroblasts.

In embodiments, about 99% of cells in an erythroid cell population described herein are positive for GPA. In an embodiment, about 98% of cells in the erythroid cell population are positive for band3. In an embodiment, about 91% of cells in the erythroid cell population are positive for alpha4 integrin. In an embodiment, about 90% of cells in the erythroid cell population are positive for both band3 and alpha4 integrin. In embodiments, the cell population is predominantly polychromatic erythroblasts and orthochromatic erythroblasts. In embodiments, the cell population is about 3% enucleated. In embodiments, the cell population is at about 6% of maximal enucleation, wherein maximal enucleation is the percentage enucleation the cell population reaches at the end of culturing. In embodiments, the cell population has an AHA intensity/incorporation value of about 2,410,000 in a BONCAT assay, e.g., as described in Example 10. In embodiments, this stage is reached when the cells have been exposed to maturation conditions for 3 days (day M3).

In embodiments, about 99.5% of cells in an erythroid cell population described herein are positive for GPA. In an embodiment, about 100% of cells in the erythroid cell population are positive for band3. In an embodiment, about 84.2% of cells in the erythroid cell population are positive for alpha4 integrin. In an embodiment, about 84.2% of cells in the erythroid cell population are positive for both band3 and alpha4 integrin. In embodiments, the cell population is predominantly orthochromatic erythroblasts and reticulocytes. In embodiments, the cell population is about 11% enucleated. In embodiments, the cell population is at about 22% of maximal enucleation. In embodiments, the cell population has an AHA intensity/incorporation value of about 1,870,000 in a BONCAT assay. In embodiments, this stage is reached when the cells have been exposed to maturation conditions for 5 days (day M5).

In embodiments, the cell population is about 34% enucleated. In embodiments, the cell population is at about 68% of maximal enucleation. In embodiments, the cell population has an AHA intensity/incorporation value of about 615,000 in a BONCAT assay. In embodiments, this stage is reached when the cells have been exposed to maturation conditions for 7 days (day M7).

In embodiments, the cell population is about 43% enucleated. In embodiments, the cell population is at about 86% of maximal enucleation. In embodiments, the cell population has an AHA intensity/incorporation value of about 189,000 in a BONCAT assay. In embodiments, this stage is reached when the cells have been exposed to maturation conditions for 9 days (day M9).

In embodiments, an erythroid cell is selected from a pro-erythroblast, early basophilic erythroblast, late basophilic erythroblast, polychromatic erythroblast, orthochromatic erythroblast, reticulocyte, or erythrocyte.

Methods of Treatment with Compositions Herein, e.g., Erythroid Cells

Methods of administering erythroid cells comprising (e.g., expressing) an exogenous RNA and/or protein are described, e.g., in WO2015/073587 and WO2015/153102, each of which is incorporated by reference in its entirety.

In embodiments, the erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In some embodiments, the erythroid cells are administered to a patient every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some aspects, the present disclosure provides a method of treating a disease or condition described herein, comprising administering to a subject in need thereof a composition described herein, e.g., an enucleated red blood cell described herein. In some embodiments, the disease or condition is cancer, an infection (e.g., a viral or bacterial infection), an inflammatory disease, an autoimmune disease, or a metabolic deficiency. In some aspects, the disclosure provides a use of an erythroid cell described herein for treating a disease or condition described herein. In some aspects, the disclosure provides a use of an erythroid cell described herein for manufacture of a medicament for treating a disease or condition described herein.

Types of cancer include acute lymphoblastic leukaemia (ALL), acute myeloid leukaemia (AML), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukaemia (CLL), chronic myeloid leukaemia (CML), colon cancer, colorectal cancer, endometrial cancer, eye cancer, gallbladder cancer, gastric cancer, gestational trophoblastic tumors (GTT), hairy cell leukaemia, head and neck cancer, Hodgkin lymphoma, kidney cancer, laryngeal cancer, leukaemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non-Hodgkin lymphoma (NHL), esophageal cancer, ovarian cancer, pancreatic cancer, penile cancer, prostate cancer, rare cancers, rectal cancer, salivary gland cancer, secondary cancers, skin cancer (non-melanoma), soft tissue sarcoma, stomach cancer, testicular cancer, thyroid cancer, unknown primary cancer, uterine cancer, vaginal cancer, and vulval cancer.

Viral infections include adenovirus, coxsackievirus, hepatitis A virus, poliovirus, Epstein-Barr virus, herpes simplex type 1, herpes simplex type 2, human cytomegalovirus, human herpesvirus type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

Inflammatory disease include bacterial sepsis, rheumatoid arthritis, age related macular degeneration (AMD), systemic lupus erythematosus (an inflammatory disorder of connective tissue), glomerulonephritis (inflammation of the capillaries of the kidney), Crohn's disease, ulcerative colitis, celiac disease, or other idiopathic inflammatory bowel diseases, and allergic asthma.

Autoimmune diseases include systemic lupus erythematosus, glomerulonephritis, rheumatoid arthritis, multiple sclerosis, and type 1 diabetes.

Metabolic deficiencies include Phenylketonuria (PKU), Adenosine Deaminase Deficiency-Severe Combined Immunodeficiency (ADA-SCID), Mitochondrial Neurogastrointestinal Encephalopathy (MNGIE), Primary Hyperoxaluria, Alkaptonuria, and Thrombotic Thrombocytopenic Purpura (TTP).

Exemplary additional features and embodiments are provided below:

1. A method of making an erythroid cell comprising a nucleic acid, e.g., an mRNA, encoding an exogenous protein, comprising:
a) providing an erythroid cell in maturation phase, and
b) contacting the erythroid cell with a nucleic acid, e.g., an mRNA, encoding the exogenous protein, under conditions that allow uptake of the nucleic acid, e.g., an mRNA, by the erythroid cell,
thereby making an erythroid cell comprising a nucleic acid, e.g., an mRNA, encoding an exogenous protein.
   2. The method of embodiment 1, wherein the erythroid cell takes up the nucleic acid, e.g., an mRNA, encoding the exogenous protein.
   3. The method of embodiment 1, comprising providing a population of erythroid cells in maturation phase and contacting a plurality of cells of the population of erythroid cells with the nucleic acid, e.g., an mRNA, encoding the exogenous protein.
   4. The method of embodiment 3, wherein the plurality of cells of the population of erythroid cells each takes up the nucleic acid, e.g., an mRNA, encoding the exogenous protein.
   5. The method of any of embodiments 1-4, wherein after uptake of the nucleic acid, e.g., an mRNA, encoding the exogenous protein, the cell or the plurality of cells express the exogenous protein.
   6. The method of embodiment 5, wherein the cell or the plurality of cells comprise the exogenous protein.
   7. The method of any of embodiments 3-6, wherein the population of erythroid cells in maturation phase is a population of cells expanded in a maturation medium for 3-7 days, e.g., 4-5 or 4-6 days.
   8. A method of manufacturing a population of reticulocytes that express an exogenous protein, the method comprising
(a) providing a population of erythroid precursor cells (e.g., CD34+ cells);
(b) culturing the population of erythroid precursor cells under differentiating conditions to provide a population of differentiating erythroid cells;
(c) contacting a plurality of cells of the population of differentiating erythroid cells with a nucleic acid, e.g., an mRNA, encoding the exogenous protein, under conditions that allow uptake of the nucleic acid, e.g., an mRNA, by the plurality of cells of the population of differentiating erythroid cells; and
(d) further culturing the plurality of cells of the population of differentiating erythroid cells to provide a population of reticulocytes,
   thereby manufacturing a population of reticulocytes that express the exogenous protein.
   9. The method of embodiment 8, wherein the further culturing comprises fewer than 3, 2, or 1 population doubling.

10. The method of any of embodiments 3-9, wherein the population of erythroid cells or the population of differentiating erythroid cells is a population of erythroid cells comprising one or more (e.g., 2, 3, 4, 5, 6, 7, 8, 9, 10, 11, 12, 13, 14, 15, 16, 17, 18, 19, 20, 21, or more) of the following properties:
i.a) 2-40%, 3-33%, 5-30%, 10-25%, or 15-20% of the cells in the population are enucleated;
i.b) greater than 0%, 0.1%, 0.2%, or 0.5%, but less than 2%, 3%, 4%, or 5% of the cells in the population are enucleated;
i.c) greater than 0%, 0.1%, 0.2%, or 0.5%, but less than 6%, 10%, 15%, 20%, or 25% of the cells in the population are enucleated;
i.d) greater than 0%, 0.1%, 0.2%, or 0.5%, but less than 30%, 35%, 40%, 45%, or 50% of the cells in the population are enucleated;
i.e) no more than 1%, 2%, 3%, 5%, 10%, 15%, or 20% of the cells in the population are enucleated;
i.f) no more than 25%, 30%, 35%, 40%, 45%, or 50% of the cells in the population are enucleated;
i.g) the population of cells has reached 6-70%, 10-60%, 20-50%, or 30-40% of maximal enucleation;
i.h) the population of cells has reached no more than 1%, 2%, 3%, 5%, 10%, 15%, or 20% of maximal enucleation;
i.i) the population of cells has reached no more than 25%, 30%, 35%, 40%, 45%, 50%, or 60% of maximal enucleation;
ii.a) the population of cells is fewer than 3, 2, or 1 population doubling from a plateau in cell division;
ii.b) the population of cells is capable of fewer than 3, 2, or 1 population doubling;
ii.c) the population will increase by no more than 1.5, 2, or 3 fold before the population reaches an enucleation level of at least 70% of cells in the population;
iii.a) at least 80%, 85%, 90%, 95%, or 99% of the cells in the population are normoblasts (e.g., polychromatic or orthochromatic normoblasts);
iii.b) at least 50%, 60%, 70%, 75%, or 79% of the cells in the population are normoblasts (e.g., polychromatic or orthochromatic normoblasts);
iii.c) 30-90%, 40-90%, 50-90%, 60-90%, or 70-90% of the cells in the population are normoblasts (e.g., polychromatic or orthochromatic normoblasts);
iii.d) at least 80%, 85%, 90%, 95%, or 99% of the cells in the population exhibit the morphology of a normoblast (e.g., a polychromatic or orthochromatic normoblast);
iii.e) at least 50%, 60%, 70%, 75%, or 79% of the cells in the population exhibit the morphology of a normoblast (e.g., a polychromatic or orthochromatic normoblast);
iii.f) 30-90%, 40-90%, 50-90%, 60-90%, or 70-90% of the cells in the population exhibit the morphology of a normoblast (e.g., a polychromatic or orthochromatic normoblast);
iv.a) the population of cells has at least 60%, 70%, 80%, or 90% of maximal translational activity;
iv.b) the population of cells has at least 20%, 30%, 40%, or 50% of maximal translational activity;
iv.c) the population of cells has a translational activity of at least 600,000, 800,000, 1,000,000, 1,200,000, 1,400,000, 1,600,000, 1,800,000, 2,000,000, 2,200,000, or 2,400,000, as measured by a BONCAT assay, e.g., by the translation assay of Example 10; or
iv. d) the population of cells has a translational activity of 600,000-2,400,000, 800,000-2,200,000, 1,000,000-2,000,000, 1,200,000-1,800,000, or 1,400,000-1,600,000, as measured by a BONCAT assay, e.g., by the translation assay of Example 10.
   11. The method of embodiment 10, wherein the population of erythroid cells or the population of differentiating erythroid cells is a population of erythroid cells comprising a property from i and a property from ii.
   12. The method of embodiment 10, wherein the population of erythroid cells or the population of differentiating erythroid cells is a population of erythroid cells comprising a property from i and a property from iii.
   13. The method of embodiment 10, wherein the population of erythroid cells or the population of differentiating erythroid cells is a population of erythroid cells comprising a property from i and a property from iv.
   14. The method of embodiment 10, wherein the population of erythroid cells or the population of differentiating erythroid cells is a population of erythroid cells comprising a property from ii and a property from iii.
   15. The method of embodiment 10, wherein the population of erythroid cells or the population of differentiating erythroid cells is a population of erythroid cells comprising a property from ii and a property from iv.
   16. The method of embodiment 10, wherein the population of erythroid cells or the population of differentiating erythroid cells is a population of erythroid cells comprising a property from iii and a property from iv.
   17. The method of embodiment 10, wherein the population of erythroid cells or the population of differentiating erythroid cells is a population of erythroid cells comprising a property from i, a property from ii, and a property from iii.
   18. The method of embodiment 10, wherein the population of erythroid cells or the population of differentiating erythroid cells is a population of erythroid cells comprising a property from i, a property from ii, and a property from iv.
   19. The method of embodiment 10, wherein the population of erythroid cells or the population of differentiating erythroid cells is a population of erythroid cells comprising a property from i, a property from iii, and a property from iv.
   20. The method of embodiment 10, wherein the population of erythroid cells or the population of differentiating erythroid cells is a population of erythroid cells comprising a property from ii, a property from iii, and a property from iv.
   21. The method of any of embodiments 10-20, wherein the population of erythroid cells or the population of differentiating erythroid cells is a population of erythroid cells comprising the following properties: i.a and ii.a.
   22. The method of any of embodiments 10-20, wherein the population of erythroid cells or the population of differentiating erythroid cells is a population of erythroid cells comprising the following properties: i.b and ii.a.
   23. The method of any of embodiments 10-20, wherein the population of erythroid cells or the population of differentiating erythroid cells is a population of erythroid cells comprising the following properties: i.c and ii.a.
   24. The method of any of embodiments 10-20, wherein the population of erythroid cells or the population of differentiating erythroid cells is a population of erythroid cells comprising the following properties: i.d and ii.a.
   25. The method of any of embodiments 10-20, wherein the population of erythroid cells or the population of differentiating erythroid cells is a population of erythroid cells comprising the following properties: i.e and ii.a.
   26. The method of any of embodiments 10-20, wherein the population of erythroid cells or the population of differentiating erythroid cells is a population of erythroid cells comprising the following properties: i.f and ii.a.
   27. The method of any of embodiments 10-20, wherein the population of erythroid cells or the population of differentiating erythroid cells is a population of erythroid cells comprising the following properties: i.g and ii.a.
   28. The method of any of embodiments 10-20, wherein the population of erythroid cells or the population of differentiating erythroid cells is a population of erythroid cells comprising the following properties: i.h and ii.a.
   29. The method of any of embodiments 10-20, wherein the population of erythroid cells or the population of differentiating erythroid cells is a population of erythroid cells comprising the following properties: i.i and ii.a.
   30. The method of any of embodiments 10-20, wherein the population of erythroid cells or the population of differentiating erythroid cells is a population of erythroid cells comprising the following properties: i.a and ii.b.
   31. The method of any of embodiments 10-20, wherein the population of erythroid cells or the population of differentiating erythroid cells is a population of erythroid cells comprising the following properties: i.b and ii.b.
   32. The method of any of embodiments 10-20, wherein the population of erythroid cells or the population of differentiating erythroid cells is a population of erythroid cells comprising the following properties: i.c and ii.b.
   33. The method of any of embodiments 10-20, wherein the population of erythroid cells or the population of differentiating erythroid cells is a population of erythroid cells comprising the following properties: i.d and ii.b.
   34. The method of any of embodiments 10-20, wherein the population of erythroid cells or the population of differentiating erythroid cells is a population of erythroid cells comprising the following properties: i.e and ii.b.
   35. The method of any of embodiments 10-20, wherein the population of erythroid cells or the population of differentiating erythroid cells is a population of erythroid cells comprising the following properties: i.f and ii.b.
   36. The method of any of embodiments 10-20, wherein the population of erythroid cells or the population of differentiating erythroid cells is a population of erythroid cells comprising the following properties: i.g and ii.b.
   37. The method of any of embodiments 10-20, wherein the population of erythroid cells or the population of differentiating erythroid cells is a population of erythroid cells comprising the following properties: i.h and ii.b.
   38. The method of any of embodiments 10-20, wherein the population of erythroid cells or the population of differentiating erythroid cells is a population of erythroid cells comprising the following properties: i.i and ii.b.
   39. The method of any of embodiments 10-20, wherein the population of erythroid cells or the population of differentiating erythroid cells is a population of erythroid cells comprising the following properties: i.a and ii.c.
   40. The method of any of embodiments 10-20, wherein the population of erythroid cells or the population of differentiating erythroid cells is a population of erythroid cells comprising the following properties: i.b and ii.c.
   41. The method of any of embodiments 10-20, wherein the population of erythroid cells or the population of differentiating erythroid cells is a population of erythroid cells comprising the following properties: i.c and ii.c.
   42. The method of any of embodiments 10-20, wherein the population of erythroid cells or the population of differentiating erythroid cells is a population of erythroid cells comprising the following properties: i.d and ii.c.
   43. The method of any of embodiments 10-20, wherein the population of erythroid cells or the population of differentiating erythroid cells is a population of erythroid cells comprising the following properties: i.e and ii.c.
   44. The method of any of embodiments 10-20, wherein the population of erythroid cells or the population of differentiating erythroid cells is a population of erythroid cells comprising the following properties: i.f and ii.c.
   45. The method of any of embodiments 10-20, wherein the population of erythroid cells or the population of differentiating erythroid cells is a population of erythroid cells comprising the following properties: i.g and ii.c.
   46. The method of any of embodiments 10-20, wherein the population of erythroid cells or the population of differentiating erythroid cells is a population of erythroid cells comprising the following properties: i.h and ii.c.
   47. The method of any of embodiments 10-20, wherein the population of erythroid cells or the population of differentiating erythroid cells is a population of erythroid cells comprising the following properties: i.i and ii.c.
   48. The method of any of embodiments 10-20, wherein the population of erythroid cells or the population of differentiating erythroid cells is a population of erythroid cells comprising the following properties: i.a and iii.a.
   49. The method of any of embodiments 10-20, wherein the population of erythroid cells or the population of differentiating erythroid cells is a population of erythroid cells comprising the following properties: i.b and iii.a.
   50. The method of any of embodiments 10-20, wherein the population of erythroid cells or the population of differentiating erythroid cells is a population of erythroid cells comprising the following properties: i.c and iii.a.
   51. The method of any of embodiments 10-20, wherein the population of erythroid cells or the population of differentiating erythroid cells is a population of erythroid cells comprising the following properties: i.d and iii.a.
   52. The method of any of embodiments 10-20, wherein the population of erythroid cells or the population of differentiating erythroid cells is a population of erythroid cells comprising the following properties: i.e and iii.a.
   53. The method of any of embodiments 10-20, wherein the population of erythroid cells or the population of differentiating erythroid cells is a population of erythroid cells comprising the following properties: i.f and iii.a.
   54. The method of any of embodiments 10-20, wherein the population of erythroid cells or the population of differentiating erythroid cells is a population of erythroid cells comprising the following properties: i.g and iii.a.
   55. The method of any of embodiments 10-20, wherein the population of erythroid cells or the population of differentiating erythroid cells is a population of erythroid cells comprising the following properties: i.h and iii.a.
   56. The method of any of embodiments 10-20, wherein the population of erythroid cells or the population of differentiating erythroid cells is a population of erythroid cells comprising the following properties: i.i and iii.a.
   57. The method of any of embodiments 10-20, wherein the population of erythroid cells or the population of differentiating erythroid cells is a population of erythroid cells comprising the following properties: i.a and iii.b.
   58. The method of any of embodiments 10-20, wherein the population of erythroid cells or the population of differentiating erythroid cells is a population of erythroid cells comprising the following properties: i.b and iii.b.
   59. The method of any of embodiments 10-20, wherein the population of erythroid cells or the population of differentiating erythroid cells is a population of erythroid cells comprising the following properties: i.c and iii.b.
   60. The method of any of embodiments 10-20, wherein the population of erythroid cells or the population of differentiating erythroid cells is a population of erythroid cells comprising the following properties: i.d and iii.b.
   61. The method of any of embodiments 10-20, wherein the population of erythroid cells or the population of differentiating erythroid cells is a population of erythroid cells comprising the following properties: i.e and iii.b.
   62. The method of any of embodiments 10-20, wherein the population of erythroid cells or the population of differentiating erythroid cells is a population of erythroid cells comprising the following properties: i.f and iii.b.
   63. The method of any of embodiments 10-20, wherein the population of erythroid cells or the population of differentiating erythroid cells is a population of erythroid cells comprising the following properties: i.g and iii.b.
   64. The method of any of embodiments 10-20, wherein the population of erythroid cells or the population of differentiating erythroid cells is a population of erythroid cells comprising the following properties: i.h and iii.b.
   65. The method of any of embodiments 10-20, wherein the population of erythroid cells or the population of differentiating erythroid cells is a population of erythroid cells comprising the following properties: i.i and iii.b.
   66. The method of any of embodiments 10-20, wherein the population of erythroid cells or the population of differentiating erythroid cells is a population of erythroid cells comprising the following properties: i.a and iii.c.
   67. The method of any of embodiments 10-20, wherein the population of erythroid cells or the population of differentiating erythroid cells is a population of erythroid cells comprising the following properties: i.b and iii.c.
   68. The method of any of embodiments 10-20, wherein the population of erythroid cells or the population of differentiating erythroid cells is a population of erythroid cells comprising the following properties: i.c and iii.c.
   69. The method of any of embodiments 10-20, wherein the population of erythroid cells or the population of differentiating erythroid cells is a population of erythroid cells comprising the following properties: i.d and iii.c.
   70. The method of any of embodiments 10-20, wherein the population of erythroid cells or the population of differentiating erythroid cells is a population of erythroid cells comprising the following properties: i.e and iii.c.
   71. The method of any of embodiments 10-20, wherein the population of erythroid cells or the population of differentiating erythroid cells is a population of erythroid cells comprising the following properties: i.f and iii.c.
   72. The method of any of embodiments 10-20, wherein the population of erythroid cells or the population of differentiating erythroid cells is a population of erythroid cells comprising the following properties: i.g and iii.c.
   73. The method of any of embodiments 10-20, wherein the population of erythroid cells or the population of differentiating erythroid cells is a population of erythroid cells comprising the following properties: i.h and iii.c.
   74. The method of any of embodiments 10-20, wherein the population of erythroid cells or the population of differentiating erythroid cells is a population of erythroid cells comprising the following properties: i.i and iii.c.
   75. The method of any of embodiments 10-20, wherein the population of erythroid cells or the population of differentiating erythroid cells is a population of erythroid cells comprising the following properties: i.a and iii.d.
   76. The method of any of embodiments 10-20, wherein the population of erythroid cells or the population of differentiating erythroid cells is a population of erythroid cells comprising the following properties: i.b and iii.d.
   77. The method of any of embodiments 10-20, wherein the population of erythroid cells or the population of differentiating erythroid cells is a population of erythroid cells comprising the following properties: i.c and iii.d.
   78. The method of any of embodiments 10-20, wherein the population of erythroid cells or the population of differentiating erythroid cells is a population of erythroid cells comprising the following properties: i.d and iii.d.
   79. The method of any of embodiments 10-20, wherein the population of erythroid cells or the population of differentiating erythroid cells is a population of erythroid cells comprising the following properties: i.e and iii.d.
   80. The method of any of embodiments 10-20, wherein the population of erythroid cells or the population of differentiating erythroid cells is a population of erythroid cells comprising the following properties: i.f and iii.d.
   81. The method of any of embodiments 10-20, wherein the population of erythroid cells or the population of differentiating erythroid cells is a population of erythroid cells comprising the following properties: i.g and iii.d.
   82. The method of any of embodiments 10-20, wherein the population of erythroid cells or the population of differentiating erythroid cells is a population of erythroid cells comprising the following properties: i.h and iii.d.
   83. The method of any of embodiments 10-20, wherein the population of erythroid cells or the population of differentiating erythroid cells is a population of erythroid cells comprising the following properties: i.i and iii.d.
   84. The method of any of embodiments 10-20, wherein the population of erythroid cells or the population of differentiating erythroid cells is a population of erythroid cells comprising the following properties: i.a and iii.e.
   85. The method of any of embodiments 10-20, wherein the population of erythroid cells or the population of differentiating erythroid cells is a population of erythroid cells comprising the following properties: i.b and iii.e.
   86. The method of any of embodiments 10-20, wherein the population of erythroid cells or the population of differentiating erythroid cells is a population of erythroid cells comprising the following properties: i.c and iii.e.
   87. The method of any of embodiments 10-20, wherein the population of erythroid cells or the population of differentiating erythroid cells is a population of erythroid cells comprising the following properties: i.d and iii.e.
   88. The method of any of embodiments 10-20, wherein the population of erythroid cells or the population of differentiating erythroid cells is a population of erythroid cells comprising the following properties: i.e and iii.e.
   89. The method of any of embodiments 10-20, wherein the population of erythroid cells or the population of differentiating erythroid cells is a population of erythroid cells comprising the following properties: i.f and iii.e.
   90. The method of any of embodiments 10-20, wherein the population of erythroid cells or the population of differentiating erythroid cells is a population of erythroid cells comprising the following properties: i.g and iii.e.
   91. The method of any of embodiments 10-20, wherein the population of erythroid cells or the population of differentiating erythroid cells is a population of erythroid cells comprising the following properties: i.h and iii.e.
   92. The method of any of embodiments 10-20, wherein the population of erythroid cells or the population of differentiating erythroid cells is a population of erythroid cells comprising the following properties: i.i and iii.e.
   93. The method of any of embodiments 10-20, wherein the population of erythroid cells or the population of differentiating erythroid cells is a population of erythroid cells comprising the following properties: i.a and iii.f.
   94. The method of any of embodiments 10-20, wherein the population of erythroid cells or the population of differentiating erythroid cells is a population of erythroid cells comprising the following properties: i.b and iii.f.
   95. The method of any of embodiments 10-20, wherein the population of erythroid cells or the population of differentiating erythroid cells is a population of erythroid cells comprising the following properties: i.c and iii.f.
   96. The method of any of embodiments 10-20, wherein the population of erythroid cells or the population of differentiating erythroid cells is a population of erythroid cells comprising the following properties: i.d and iii.f.
   97. The method of any of embodiments 10-20, wherein the population of erythroid cells or the population of differentiating erythroid cells is a population of erythroid cells comprising the following properties: i.e and iii.f.
   98. The method of any of embodiments 10-20, wherein the population of erythroid cells or the population of differentiating erythroid cells is a population of erythroid cells comprising the following properties: i.f and iii.f.
   99. The method of any of embodiments 10-20, wherein the population of erythroid cells or the population of differentiating erythroid cells is a population of erythroid cells comprising the following properties: i.g and iii.f.
   100. The method of any of embodiments 10-20, wherein the population of erythroid cells or the population of differentiating erythroid cells is a population of erythroid cells comprising the following properties: i.h and iii.f.
   101. The method of any of embodiments 10-20, wherein the population of erythroid cells or the population of differentiating erythroid cells is a population of erythroid cells comprising the following properties: i.i and iii.f.
   102. The method of any of embodiments 10-20, wherein the population of erythroid cells or the population of differentiating erythroid cells is a population of erythroid cells comprising the following properties: i.a and iv.a.
   103. The method of any of embodiments 10-20, wherein the population of erythroid cells or the population of differentiating erythroid cells is a population of erythroid cells comprising the following properties: i.b and iv.a.
   104. The method of any of embodiments 10-20, wherein the population of erythroid cells or the population of differentiating erythroid cells is a population of erythroid cells comprising the following properties: i.c and iv.a.
   105. The method of any of embodiments 10-20, wherein the population of erythroid cells or the population of differentiating erythroid cells is a population of erythroid cells comprising the following properties: i.d and iv.a.
   106. The method of any of embodiments 10-20, wherein the population of erythroid cells or the population of differentiating erythroid cells is a population of erythroid cells comprising the following properties: i.e and iv.a.
   107. The method of any of embodiments 10-20, wherein the population of erythroid cells or the population of differentiating erythroid cells is a population of erythroid cells comprising the following properties: i.f and iv.a.
   108. The method of any of embodiments 10-20, wherein the population of erythroid cells or the population of differentiating erythroid cells is a population of erythroid cells comprising the following properties: i.g and iv.a.
   109. The method of any of embodiments 10-20, wherein the population of erythroid cells or the population of differentiating erythroid cells is a population of erythroid cells comprising the following properties: i.h and iv.a.
   110. The method of any of embodiments 10-20, wherein the population of erythroid cells or the population of differentiating erythroid cells is a population of erythroid cells comprising the following properties: i.i and iv.a.
   111. The method of any of embodiments 10-20, wherein the population of erythroid cells or the population of differentiating erythroid cells is a population of erythroid cells comprising the following properties: i.a and iv.b.
   112. The method of any of embodiments 10-20, wherein the population of erythroid cells or the population of differentiating erythroid cells is a population of erythroid cells comprising the following properties: i.b and iv.b.
   113. The method of any of embodiments 10-20, wherein the population of erythroid cells or the population of differentiating erythroid cells is a population of erythroid cells comprising the following properties: i.c and iv.b.
   114. The method of any of embodiments 10-20, wherein the population of erythroid cells or the population of differentiating erythroid cells is a population of erythroid cells comprising the following properties: i.d and iv.b.
   115. The method of any of embodiments 10-20, wherein the population of erythroid cells or the population of differentiating erythroid cells is a population of erythroid cells comprising the following properties: i.e and iv.b.
   116. The method of any of embodiments 10-20, wherein the population of erythroid cells or the population of differentiating erythroid cells is a population of erythroid cells comprising the following properties: i.f and iv.b.
   117. The method of any of embodiments 10-20, wherein the population of erythroid cells or the population of differentiating erythroid cells is a population of erythroid cells comprising the following properties: i.g and iv.b.
   118. The method of any of embodiments 10-20, wherein the population of erythroid cells or the population of differentiating erythroid cells is a population of erythroid cells comprising the following properties: i.h and iv.b.
   119. The method of any of embodiments 10-20, wherein the population of erythroid cells or the population of differentiating erythroid cells is a population of erythroid cells comprising the following properties: i.i and iv.b.
   120. The method of any of embodiments 10-20, wherein the population of erythroid cells or the population of differentiating erythroid cells is a population of erythroid cells comprising the following properties: i.a and iv.c.
   121. The method of any of embodiments 10-20, wherein the population of erythroid cells or the population of differentiating erythroid cells is a population of erythroid cells comprising the following properties: i.b and iv.c.
   122. The method of any of embodiments 10-20, wherein the population of erythroid cells or the population of differentiating erythroid cells is a population of erythroid cells comprising the following properties: i.c and iv.c.
   123. The method of any of embodiments 10-20, wherein the population of erythroid cells or the population of differentiating erythroid cells is a population of erythroid cells comprising the following properties: i.d and iv.c.
   124. The method of any of embodiments 10-20, wherein the population of erythroid cells or the population of differentiating erythroid cells is a population of erythroid cells comprising the following properties: i.e and iv.c.
   125. The method of any of embodiments 10-20, wherein the population of erythroid cells or the population of differentiating erythroid cells is a population of erythroid cells comprising the following properties: i.f and iv.c.
   126. The method of any of embodiments 10-20, wherein the population of erythroid cells or the population of differentiating erythroid cells is a population of erythroid cells comprising the following properties: i.g and iv.c.
   127. The method of any of embodiments 10-20, wherein the population of erythroid cells or the population of differentiating erythroid cells is a population of erythroid cells comprising the following properties: i.h and iv.c.
   128. The method of any of embodiments 10-20, wherein the population of erythroid cells or the population of differentiating erythroid cells is a population of erythroid cells comprising the following properties: i.i and iv.c.
   129. The method of any of embodiments 10-20, wherein the population of erythroid cells or the population of differentiating erythroid cells is a population of erythroid cells comprising the following properties: i.a and iv.d.
   130. The method of any of embodiments 10-20, wherein the population of erythroid cells or the population of differentiating erythroid cells is a population of erythroid cells comprising the following properties: i.b and iv.d.
   131. The method of any of embodiments 10-20, wherein the population of erythroid cells or the population of differentiating erythroid cells is a population of erythroid cells comprising the following properties: i.c and iv.d.
   132. The method of any of embodiments 10-20, wherein the population of erythroid cells or the population of differentiating erythroid cells is a population of erythroid cells comprising the following properties: i.d and iv.d.
   133. The method of any of embodiments 10-20, wherein the population of erythroid cells or the population of differentiating erythroid cells is a population of erythroid cells comprising the following properties: i.e and iv.d.
   134. The method of any of embodiments 10-20, wherein the population of erythroid cells or the population of differentiating erythroid cells is a population of erythroid cells comprising the following properties: i.f and iv.d.
   135. The method of any of embodiments 10-20, wherein the population of erythroid cells or the population of differentiating erythroid cells is a population of erythroid cells comprising the following properties: i.g and iv.d.
   136. The method of any of embodiments 10-20, wherein the population of erythroid cells or the population of differentiating erythroid cells is a population of erythroid cells comprising the following properties: i.h and iv.d.
   137. The method of any of embodiments 10-20, wherein the population of erythroid cells or the population of differentiating erythroid cells is a population of erythroid cells comprising the following properties: i.i and iv.d.
   138. The method of any of embodiments 10-20, wherein the population of erythroid cells or the population of differentiating erythroid cells is a population of erythroid cells comprising the following properties: iii.a and ii.a.
   139. The method of any of embodiments 10-20, wherein the population of erythroid cells or the population of differentiating erythroid cells is a population of erythroid cells comprising the following properties: iii.b and ii.a.
   140. The method of any of embodiments 10-20, wherein the population of erythroid cells or the population of differentiating erythroid cells is a population of erythroid cells comprising the following properties: iii.c and ii.a.
   141. The method of any of embodiments 10-20, wherein the population of erythroid cells or the population of differentiating erythroid cells is a population of erythroid cells comprising the following properties: iii.d and ii.a.
   142. The method of any of embodiments 10-20, wherein the population of erythroid cells or the population of differentiating erythroid cells is a population of erythroid cells comprising the following properties: iii.e and ii.a.
   143. The method of any of embodiments 10-20, wherein the population of erythroid cells or the population of differentiating erythroid cells is a population of erythroid cells comprising the following properties: iii.f and ii.a.
   144. The method of any of embodiments 10-20, wherein the population of erythroid cells or the population of differentiating erythroid cells is a population of erythroid cells comprising the following properties: iii.a and ii.b.
   145. The method of any of embodiments 10-20, wherein the population of erythroid cells or the population of differentiating erythroid cells is a population of erythroid cells comprising the following properties: iii.b and ii.b.
   146. The method of any of embodiments 10-20, wherein the population of erythroid cells or the population of differentiating erythroid cells is a population of erythroid cells comprising the following properties: iii.c and ii.b.
   147. The method of any of embodiments 10-20, wherein the population of erythroid cells or the population of differentiating erythroid cells is a population of erythroid cells comprising the following properties: iii.d and ii.b.
   148. The method of any of embodiments 10-20, wherein the population of erythroid cells or the population of differentiating erythroid cells is a population of erythroid cells comprising the following properties: iii.e and ii.b.
   149. The method of any of embodiments 10-20, wherein the population of erythroid cells or the population of differentiating erythroid cells is a population of erythroid cells comprising the following properties: iii.f and ii.b.
   150. The method of any of embodiments 10-20, wherein the population of erythroid cells or the population of differentiating erythroid cells is a population of erythroid cells comprising the following properties: iii.a and ii.c.
   151. The method of any of embodiments 10-20, wherein the population of erythroid cells or the population of differentiating erythroid cells is a population of erythroid cells comprising the following properties: iii.b and ii.c.
   152. The method of any of embodiments 10-20, wherein the population of erythroid cells or the population of differentiating erythroid cells is a population of erythroid cells comprising the following properties: iii.c and ii.c.
   153. The method of any of embodiments 10-20, wherein the population of erythroid cells or the population of differentiating erythroid cells is a population of erythroid cells comprising the following properties: iii.d and ii.c.
   154. The method of any of embodiments 10-20, wherein the population of erythroid cells or the population of differentiating erythroid cells is a population of erythroid cells comprising the following properties: iii.e and ii.c.
   155. The method of any of embodiments 10-20, wherein the population of erythroid cells or the population of differentiating erythroid cells is a population of erythroid cells comprising the following properties: iii.f and ii.c.
   156. The method of any of embodiments 10-20, wherein the population of erythroid cells or the population of differentiating erythroid cells is a population of erythroid cells comprising the following properties: iii.a and iv.a.
   157. The method of any of embodiments 10-20, wherein the population of erythroid cells or the population of differentiating erythroid cells is a population of erythroid cells comprising the following properties: iii.b and iv.a.
   158. The method of any of embodiments 10-20, wherein the population of erythroid cells or the population of differentiating erythroid cells is a population of erythroid cells comprising the following properties: iii.c and iv.a.
   159. The method of any of embodiments 10-20, wherein the population of erythroid cells or the population of differentiating erythroid cells is a population of erythroid cells comprising the following properties: iii.d and iv.a.
   160. The method of any of embodiments 10-20, wherein the population of erythroid cells or the population of differentiating erythroid cells is a population of erythroid cells comprising the following properties: iii.e and iv.a.
   161. The method of any of embodiments 10-20, wherein the population of erythroid cells or the population of differentiating erythroid cells is a population of erythroid cells comprising the following properties: iii.f and iv.a.
   162. The method of any of embodiments 10-20, wherein the population of erythroid cells or the population of differentiating erythroid cells is a population of erythroid cells comprising the following properties: iii.a and iv.b.
   163. The method of any of embodiments 10-20, wherein the population of erythroid cells or the population of differentiating erythroid cells is a population of erythroid cells comprising the following properties: iii.b and iv.b.
   164. The method of any of embodiments 10-20, wherein the population of erythroid cells or the population of differentiating erythroid cells is a population of erythroid cells comprising the following properties: iii.c and iv.b.
   165. The method of any of embodiments 10-20, wherein the population of erythroid cells or the population of differentiating erythroid cells is a population of erythroid cells comprising the following properties: iii.d and iv.b.
   166. The method of any of embodiments 10-20, wherein the population of erythroid cells or the population of differentiating erythroid cells is a population of erythroid cells comprising the following properties: iii.e and iv.b.
   167. The method of any of embodiments 10-20, wherein the population of erythroid cells or the population of differentiating erythroid cells is a population of erythroid cells comprising the following properties: iii.f and iv.b.
   168. The method of any of embodiments 10-20, wherein the population of erythroid cells or the population of differentiating erythroid cells is a population of erythroid cells comprising the following properties: iii.a and iv.c.
   169. The method of any of embodiments 10-20, wherein the population of erythroid cells or the population of differentiating erythroid cells is a population of erythroid cells comprising the following properties: iii.b and iv.c.
   170. The method of any of embodiments 10-20, wherein the population of erythroid cells or the population of differentiating erythroid cells is a population of erythroid cells comprising the following properties: iii.c and iv.c.
   171. The method of any of embodiments 10-20, wherein the population of erythroid cells or the population of differentiating erythroid cells is a population of erythroid cells comprising the following properties: iii.d and iv.c.
   172. The method of any of embodiments 10-20, wherein the population of erythroid cells or the population of differentiating erythroid cells is a population of erythroid cells comprising the following properties: iii.e and iv.c.
   173. The method of any of embodiments 10-20, wherein the population of erythroid cells or the population of differentiating erythroid cells is a population of erythroid cells comprising the following properties: iii.f and iv.c.
   174. The method of any of embodiments 10-20, wherein the population of erythroid cells or the population of differentiating erythroid cells is a population of erythroid cells comprising the following properties: iii.a and iv.d.
   175. The method of any of embodiments 10-20, wherein the population of erythroid cells or the population of differentiating erythroid cells is a population of erythroid cells comprising the following properties: iii.b and iv.d.
   176. The method of any of embodiments 10-20, wherein the population of erythroid cells or the population of differentiating erythroid cells is a population of erythroid cells comprising the following properties: iii.c and iv.d.
   177. The method of any of embodiments 10-20, wherein the population of erythroid cells or the population of differentiating erythroid cells is a population of erythroid cells comprising the following properties: iii.d and iv.d.
   178. The method of any of embodiments 10-20, wherein the population of erythroid cells or the population of differentiating erythroid cells is a population of erythroid cells comprising the following properties: iii.e and iv.d.
   179. The method of any of embodiments 10-20, wherein the population of erythroid cells or the population of differentiating erythroid cells is a population of erythroid cells comprising the following properties: iii.f and iv.d.
   180. The method of any of embodiments 10-20, wherein the population of erythroid cells or the population of differentiating erythroid cells is a population of erythroid cells comprising the following properties: ii.a and iv.a.
   181. The method of any of embodiments 10-20, wherein the population of erythroid cells or the population of differentiating erythroid cells is a population of erythroid cells comprising the following properties: ii.b and iv.a.
   182. The method of any of embodiments 10-20, wherein the population of erythroid cells or the population of differentiating erythroid cells is a population of erythroid cells comprising the following properties: ii.c and iv.a.
   183. The method of any of embodiments 10-20, wherein the population of erythroid cells or the population of differentiating erythroid cells is a population of erythroid cells comprising the following properties: ii.a and iv.b.
   184. The method of any of embodiments 10-20, wherein the population of erythroid cells or the population of differentiating erythroid cells is a population of erythroid cells comprising the following properties: ii.b and iv.b.
   185. The method of any of embodiments 10-20, wherein the population of erythroid cells or the population of differentiating erythroid cells is a population of erythroid cells comprising the following properties: ii.c and iv.b.
   186. The method of any of embodiments 10-20, wherein the population of erythroid cells or the population of differentiating erythroid cells is a population of erythroid cells comprising the following properties: ii.a and iv.c.
   187. The method of any of embodiments 10-20, wherein the population of erythroid cells or the population of differentiating erythroid cells is a population of erythroid cells comprising the following properties: ii.b and iv.c.
   188. The method of any of embodiments 10-20, wherein the population of erythroid cells or the population of differentiating erythroid cells is a population of erythroid cells comprising the following properties: ii.c and iv.c.
   189. The method of any of embodiments 10-20, wherein the population of erythroid cells or the population of differentiating erythroid cells is a population of erythroid cells comprising the following properties: ii.a and iv.d.
   190. The method of any of embodiments 10-20, wherein the population of erythroid cells or the population of differentiating erythroid cells is a population of erythroid cells comprising the following properties: ii.b and iv.d.
   191. The method of any of embodiments 10-20, wherein the population of erythroid cells or the population of differentiating erythroid cells is a population of erythroid cells comprising the following properties: ii.c and iv.d.
   192. The method of any of embodiments 3-191, wherein the population of erythroid cells or the population of differentiating erythroid cells is a population of erythroid cells comprising the following property: 84-99%, 85-95%, or about 90% of the cells in the population are GPA-positive, e.g., as measured by a flow cytometry assay, e.g., a flow cytometry assay of Example 10.
   193. The method of any of embodiments 3-191, wherein the population of erythroid cells or the population of differentiating erythroid cells is a population of erythroid cells comprising the following property: at least 84%, 85%, 90%, 95%, or 99% of the cells in the population are GPA-positive, e.g., as measured by a flow cytometry assay, e.g., a flow cytometry assay of Example 10.
   194. The method of any of embodiments 3-191, wherein the population of erythroid cells or the population of differentiating erythroid cells is a population of erythroid cells comprising the following property: 54-99%, 55-98%, 60-95%, 65-90%, 70-85%, or 75-80% of the cells in the population are band3-positive, e.g., as measured by a flow cytometry assay, e.g., a flow cytometry assay of Example 10.
   195. The method of any of embodiments 3-191, wherein the population of erythroid cells or the population of differentiating erythroid cells is a population of erythroid cells comprising the following property: at least 54%, 55%, 60%, 65%, 70%, 75%, 80%, 85%, 90%, 95%, 98%, or 99% of the cells in the population are band3-positive, e.g., as measured by a flow cytometry assay, e.g., a flow cytometry assay of Example 10.
   196. The method of any of embodiments 3-191, wherein the population of erythroid cells or the population of differentiating erythroid cells is a population of erythroid cells comprising the following property: 96-100%, 97-99%, or about 98% of the cells in the population are alpha4 integrin-positive, e.g., as measured by a flow cytometry assay, e.g., a flow cytometry assay of Example 10.
   197. The method of any of embodiments 3-191, wherein the population of erythroid cells or the population of differentiating erythroid cells is a population of erythroid cells comprising the following property: at least 95%, 96%, 97%, 98%, or 99% of the cells in the population are alpha4 integrin-positive, e.g., as measured by a flow cytometry assay, e.g., a flow cytometry assay of Example 10.
   198. The method of any of embodiments 3-197, wherein prior to or after contacting the plurality of cells with the nucleic acid, e.g., an mRNA, encoding the exogenous protein, the plurality of cells are separated from the population of erythroid cells or the population of differentiating erythroid cells, e.g., the plurality of cells are separated from the population based on enucleation status (e.g., the plurality of cells are nucleated cells and the rest of the population are enucleated cells).
   199. The method of any of embodiments 3-197, comprising prior to or after contacting the plurality of cells with the nucleic acid, e.g., an mRNA, encoding the exogenous protein, synchronizing the population of erythroid cells or the population of differentiating erythroid cells, e.g., by arresting the growth, development, hemoglobin synthesis, or the process of enucleation of the population, e.g., by incubating the population with an inhibitor of enucleation (e.g., an inhibitor of histone deacetylase (HDAC), an inhibitor of mitogen-activated protein kinase (MAPK), an inhibitor of cyclin-dependent kinase (CDK), or a proteasome inhibitor).
   200. The method of embodiment 199, wherein arresting occurs prior to enucleation of more than 1, 2, 3, 4, 5, 6, 7, 8, 9 or 10% of the cells in the population.
   201. A method of manufacturing a population of reticulocytes that express an exogenous protein, the method comprising:
(e) providing a population of erythroid precursor cells (e.g., CD34+ cells);
(f) culturing the population of erythroid precursor cells under differentiating conditions to provide a population of differentiating erythroid cells;
(g) contacting the differentiating erythroid cells with an mRNA encoding the exogenous protein, under conditions that allow uptake of the mRNA by the differentiating erythroid cells, wherein the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated); and
(h) further culturing the differentiating erythroid cells to provide a population of reticulocytes, thereby manufacturing a population of reticulocytes that express the exogenous protein.
   202. The method of embodiment 201, wherein the further culturing comprises fewer than 3, 2, or 1 population doubling.
   203. The method of embodiment 201 or 202, wherein the contacting is performed when at least 50% (at least 60%, 70%, 75%, 80%, 90%, or 95%) of the differentiating erythroid cells exhibit the morphology of a normoblast (e.g., a polychromatic or orthochromatic normoblast).
   204. A method of manufacturing a population of reticulocytes that express an exogenous protein, comprising (a) providing a population of erythroid precursor cells, (b) culturing the population of erythroid precursor cells under differentiating conditions to provide a population of differentiating erythroid cells, (c) contacting the differentiating erythroid cells with an mRNA encoding the exogenous protein, wherein the improvement comprises: the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated).
   205. The method of embodiment 204, wherein the contacting is performed when the population of differentiating erythroid cells has fewer than 3, 2, or 1 population doubling before a plateau in cell division.
   206. The method of embodiment 204 or 205, wherein the contacting is performed when at least 50% (at least 60%, 70%, 75%, 80%, 90%, or 95%) of the differentiating erythroid cells exhibit the morphology of a normoblast (e.g., a polychromatic or orthochromatic normoblast).
   207. An erythroid cell, e.g., an enucleated erythroid cell, comprising:

an exogenous mRNA comprising a coding region operatively linked to a heterologous untranslated region (UTR), wherein the heterologous UTR comprises a regulatory element.
208. An erythroid cell, e.g., an enucleated erythroid cell, comprising an exogenous mRNA that comprises one or more chemically modified nucleotides of Table 1, one or more chemical backbone modifications of Table 2, one or more chemically modified caps of Table 3, or any combination thereof.
   209. A method of producing an erythroid cell, e.g., enucleated erythroid cell, comprising:
a) contacting an erythroid cell, e.g., a nucleated erythroid cell, with an exogenous mRNA comprising a coding region operatively linked to a heterologous UTR comprising a regulatory element, (e.g., isolated RNA or in vitro transcribed RNA), and
b) maintaining the contacted erythroid cell under conditions suitable for uptake of the exogenous mRNA,
thereby producing the erythroid cell, e.g., an enucleated erythroid cell.
   210. A method of producing an erythroid cell, e.g., enucleated erythroid cell, comprising:
a) contacting an erythroid cell, e.g., a nucleated erythroid cell, with an exogenous mRNA comprising one or more chemically modified nucleotides of Table 1, one or more chemical backbone modifications of Table 2, one or more chemically modified caps of Table 3, or any combination thereof; and
b) maintaining the contacted erythroid cell under conditions suitable for uptake of the exogenous mRNA, thereby producing the erythroid cell, e.g., an enucleated erythroid cell.
   211. A method of producing an exogenous protein in an enucleated erythroid cell:
a) providing an erythroid cell, e.g., a nucleated erythroid cell, comprising an exogenous mRNA comprising a coding region operatively linked to a heterologous UTR comprising a regulatory element, (e.g., isolated RNA or in vitro transcribed RNA), and
b) culturing the erythroid cell under conditions suitable for production of the exogenous protein,
thereby producing the exogenous protein.
   212. A method of producing an exogenous protein in an enucleated erythroid cell:
a) providing an erythroid cell, e.g., a nucleated erythroid cell, comprising one or more chemically modified nucleotides of Table 1, one or more chemical backbone modifications of Table 2, one or more chemically modified caps of Table 3, or any combination thereof, and
b) culturing the erythroid cell under conditions suitable for production of the exogenous protein,
thereby producing the exogenous protein.
   213. A method of providing a subject with an exogenous protein, providing a subject with an enucleated erythroid cell which can produce an exogenous protein, or treating a subject, comprising administering to the subject:
an erythroid cell, e.g., a nucleated erythroid cell, comprising an exogenous mRNA comprising a coding region operatively linked to a heterologous UTR comprising a regulatory element, (e.g., isolated RNA or in vitro transcribed RNA),
thereby providing a subject with an exogenous protein, providing a subject with an enucleated erythroid cell which can produce an exogenous protein, or treating a subject.
   214. A method of providing a subject with an exogenous protein, providing a subject with an enucleated erythroid cell which can produce an exogenous protein, or treating a subject, comprising administering to the subject:
an erythroid cell, e.g., a nucleated erythroid cell, comprising an exogenous mRNA comprising one or more chemically modified nucleotides of Table 1, one or more chemical backbone modifications of Table 2, one or more chemically modified caps of Table 3, or any combination thereof,
thereby providing a subject with an exogenous protein, providing a subject with an enucleated erythroid cell which can produce an exogenous protein, or treating a subject.
   215. A method of evaluating an erythroid cell, e.g., enucleated erythroid cell (or a batch of such cells) comprising:
a) providing an erythroid cell, e.g., a nucleated erythroid cell, comprising an exogenous mRNA comprising a coding region operatively linked to a heterologous UTR comprising a regulatory element (or a batch of such cells), and
b) evaluating the erythroid cell, e.g., the nucleated erythroid cell (or batch of such cells) for a preselected parameter,
thereby evaluating the erythroid cell, e.g., enucleated erythroid cell (or a batch of such cells).
   216. A method of evaluating an erythroid cell, e.g., enucleated erythroid cell (or a batch of such cells) comprising:
a) providing an erythroid cell, e.g., a nucleated erythroid cell, comprising one or more chemically modified nucleotides of Table 1, one or more chemical backbone modifications of Table 2, one or more chemically modified caps of Table 3, or any combination thereof, and
b) evaluating the erythroid cell, e.g., the nucleated erythroid cell (or batch of such cells) for a preselected parameter,
thereby evaluating the erythroid cell, e.g., enucleated erythroid cell (or a batch of such cells).
   217. The method of embodiment 207, wherein at least 50%, 60%, 70%, 80%, 85%, 90%, or 95% of the cells in the population comprise the exogenous protein, e.g., 5 days after contacting with the mRNA.
   218. The method of embodiment 207, wherein the cells in the population comprise at least 1,000, 2,000, 5,000, 10,000, 20,000, 50,000, or 100,000 copies of the exogenous protein, e.g., 5 days after the contacting with the mRNA.
   219. The method of embodiment 207, wherein the cells comprise at least 1,000, 2,000, 5,000, 10,000, 20,000, 50,000, or 100,000 copies of the exogenous protein for at least 5, 6, 7, 8, 9, 10, 11, 12, 13, 14, or 15 days after contacting with the mRNA.
   220. A method of making an erythroid cell comprising an mRNA that encodes an exogenous protein, comprising:
providing a reaction mixture comprising an erythroid cell and an mRNA encoding the exogenous protein, under conditions which inhibit degradation of mRNA, e.g., by inclusion in the reaction mixture a ribonuclease inhibitor, and
maintaining the reaction mixture under conditions that allow uptake of the mRNA by the erythroid cell,
thereby making an erythroid cell comprising an mRNA encoding an exogenous protein.
   221. The method of embodiment 220, comprising providing a population of erythroid cells and contacting the population with the mRNA encoding the exogenous protein.
   222. The method of embodiment 220 or 221, wherein a plurality of erythroid cells of the population each takes up an mRNA encoding the exogenous protein.
   223. The method of any of embodiments 220-222, wherein the cell or plurality of cells express the exogenous protein.
   224. The method of any of embodiments 220-223, wherein the cell or plurality of cells comprise the exogenous protein.
   225. The method of any of embodiments 220-224, which further comprises electroporating the cell or population of cells.
   226. The method of any of embodiments 220-225, which further comprises contacting a population of erythroid cells with a ribonuclease inhibitor.
   227. The method of any of embodiments 220-226, which comprises contacting the population of cells with the ribonuclease inhibitor before, during, or after contacting the cells with the mRNA.
   228. The method of any of embodiments 220-227, which comprises contacting the cells with the ribonuclease inhibitor at day 4, 5, or 6 of maturation phase. 229. The method of any of embodiments 220-228, wherein the cell is in maturation phase.
   230. The method of any of embodiments 220-229, which comprises contacting the cells with the ribonuclease inhibitor at a time when the cells comprise one or more (e.g., 2, 3, 4, 5, 6, 7, 8, 9, 10, 11, 12, 13, 14, 15, 16, 17, 18, 19, 20, 21, or more) of the following properties:
i.a) 2-40%, 3-33%, 5-30%, 10-25%, or 15-20% of the cells in the population are enucleated;
i.b) greater than 0%, 0.1%, 0.2%, or 0.5%, but less than 2%, 3%, 4%, or 5% of the cells in the population are enucleated;
i.c) greater than 0%, 0.1%, 0.2%, or 0.5%, but less than 6%, 10%, 15%, 20%, or 25% of the cells in the population are enucleated;
i.d) greater than 0%, 0.1%, 0.2%, or 0.5%, but less than 30%, 35%, 40%, 45%, or 50% of the cells in the population are enucleated;
i.e) no more than 1%, 2%, 3%, 5%, 10%, 15%, or 20% of the cells in the population are enucleated;
i.f) no more than 25%, 30%, 35%, 40%, 45%, or 50% of the cells in the population are enucleated;
i.g) the population of cells has reached 6-70%, 10-60%, 20-50%, or 30-40% of maximal enucleation;
i.h) the population of cells has reached no more than 1%, 2%, 3%, 5%, 10%, 15%, or 20% of maximal enucleation;
i.i) the population of cells has reached no more than 25%, 30%, 35%, 40%, 45%, 50%, or 60% of maximal enucleation;
ii.a) the population of cells is fewer than 3, 2, or 1 population doubling from a plateau in cell division;
ii.b) the population of cells is capable of fewer than 3, 2, or 1 population doubling;
ii.c) the population will increase by no more than 1.5, 2, or 3 fold before the population reaches an enucleation level of at least 70% of cells in the population;
iii.a) at least 80%, 85%, 90%, 95%, or 99% of the cells in the population are normoblasts (e.g., polychromatic or orthochromatic normoblasts);
iii.b) at least 50%, 60%, 70%, 75%, or 79% of the cells in the population are normoblasts (e.g., polychromatic or orthochromatic normoblasts);
iii.c) 30-90%, 40-90%, 50-90%, 60-90%, or 70-90% of the cells in the population are normoblasts (e.g., polychromatic or orthochromatic normoblasts);
iii.d) at least 80%, 85%, 90%, 95%, or 99% of the cells in the population exhibit the morphology of a normoblast (e.g., a polychromatic or orthochromatic normoblast);
iii.e) at least 50%, 60%, 70%, 75%, or 79% of the cells in the population exhibit the morphology of a normoblast (e.g., a polychromatic or orthochromatic normoblast);
iii.f) 30-90%, 40-90%, 50-90%, 60-90%, or 70-90% of the cells in the population exhibit the morphology of a normoblast (e.g., a polychromatic or orthochromatic normoblast);
iv.a) the population of cells has at least 60%, 70%, 80%, or 90% of maximal translational activity;
iv.b) the population of cells has at least 20%, 30%, 40%, or 50% of maximal translational activity;
iv.c) the population of cells has a translational activity of at least 600,000, 800,000, 1,000,000, 1,200,000, 1,400,000, 1,600,000, 1,800,000, 2,000,000, 2,200,000, or 2,400,000, as measured by a BONCAT assay, e.g., by the translation assay of Example 10; or
iv. d) the population of cells has a translational activity of 600,000-2,400,000, 800,000-2,200,000, 1,000,000-2,000,000, 1,200,000-1,800,000, or 1,400,000-1,600,000, as measured by a BONCAT assay, e.g., by the translation assay of Example 10.
   231. The method of embodiment 230, wherein the population of erythroid cells or the population of differentiating erythroid cells is a population of erythroid cells comprising a property from i and a property from ii.
   232. The method of embodiment 230, wherein the population of erythroid cells or the population of differentiating erythroid cells is a population of erythroid cells comprising a property from i and a property from iii.
   233. The method of embodiment 230, wherein the population of erythroid cells or the population of differentiating erythroid cells is a population of erythroid cells comprising a property from i and a property from iv.
   234. The method of embodiment 230, wherein the population of erythroid cells or the population of differentiating erythroid cells is a population of erythroid cells comprising a property from ii and a property from iii.
   235. The method of embodiment 230, wherein the population of erythroid cells or the population of differentiating erythroid cells is a population of erythroid cells comprising a property from ii and a property from iv.
   236. The method of embodiment 230, wherein the population of erythroid cells or the population of differentiating erythroid cells is a population of erythroid cells comprising a property from iii and a property from iv.
   237. The method of embodiment 230, wherein the population of erythroid cells or the population of differentiating erythroid cells is a population of erythroid cells comprising a property from i, a property from ii, and a property from iii.
   238. The method of embodiment 230, wherein the population of erythroid cells or the population of differentiating erythroid cells is a population of erythroid cells comprising a property from i, a property from ii, and a property from iv.
   239. The method of embodiment 230, wherein the population of erythroid cells or the population of differentiating erythroid cells is a population of erythroid cells comprising a property from i, a property from iii, and a property from iv.
   240. The method of embodiment 230, wherein the population of erythroid cells or the population of differentiating erythroid cells is a population of erythroid cells comprising a property from ii, a property from iii, and a property from iv.
   241. The method of any of embodiments 220-240, which comprises contacting the cells with the ribonuclease inhibitor at a time when (e.g., by a flow cytometry assay, e.g., a flow cytometry assay of Example 10) the cells comprise one or more (e.g., 2, 3, 4, 5, or more) of the following properties:
84-99%, 85-95%, or about 90% of the cells in the population are GPA-positive;
at least 84%, 85%, 90%, 95%, or 99% of the cells in the population are GPA-positive;
54-99%, 55-98%, 60-95%, 65-90%, 70-85%, or 75-80% of the cells in the population are band3-positive;
at least 54%, 55%, 60%, 65%, 70%, 75%, 80%, 85%, 90%, 95%, 98%, or 99% of the cells in the population are band3-positive;
96-100%, 97-99%, or about 98% of the cells in the population are alpha4 integrin-positive; or
at least 95%, 96%, 97%, 98%, or 99% of the cells in the population are alpha4 integrin-positive.
   242. The method of any of embodiments 220-241, wherein the mRNA is in vitro transcribed mRNA.
   243. The method of any of embodiments 220-242, wherein at least 80%, 85%, 90%, or 95% of the cells of the population are viable 5 days after the cells are contacted with the mRNA.
   244. The method of any of embodiments 220-243, wherein at least 10%, 20%, 30%, 40%, 50%, 60%, 70%, 80%, or 90% of the cells of the population are enucleated 5 days after the cells are contacted with the mRNA.
   245. The method of any of embodiments 220-244, wherein the proportion of cells that are enucleated 5 days after the cells are contacted with the mRNA is at least 50%, 60%, 70%, 80%, 90%, or 95% of the proportion of cells that are enucleated in an otherwise similar population of cells not treated with the ribonuclease inhibitor.
   246. The method of any of embodiments 220-245, wherein the population of cells comprises at least 1×10⁶, 2×10⁶, 5×10⁶, 1×10⁷, 2×10⁷, 5×10⁷, or 1×10⁸ cells at the time the cells are contacted with the mRNA.
   247. The method of any of embodiments 220-246, wherein the population of cells expands by at least 10%, 20%, 30%, 40%, 50%, 60%, 70%, 80%, or 90% within 5 days after the cells are contacted with the mRNA.
   248. The method of any of embodiments 220-247, wherein at least 50%, 60%, 70%, 80%, 85%, 90%, or 95% of the cells of the population express the exogenous protein, e.g., 5 days after the cells are contacted with the mRNA.
   249. The method of any of embodiments 220-248, wherein at least 50%, 60%, 70%, 80%, 85%, 90%, or 95% of the cells of the population comprise at least 1,000, 2,000, 5,000, 10,000, 20,000, 50,000, or 100,000 copies of the exogenous protein, e.g., 5 days after the cells are contacted with the mRNA.
   250. The method of any of embodiments 220-249, wherein the population of cells comprises at least 10%, 20%, 30%, 40%, 50%, 60%, 70%, 80%, or 90% more, or at least 2-fold, 3-fold, 4-fold, or 5-fold more of the exogenous protein than an otherwise similar population of cells not treated with the ribonuclease inhibitor.
   251. A reaction mixture comprising: i) an erythroid cell, ii) an mRNA comprising an exogenous protein and iii) a ribonuclease inhibitor.
   252. The reaction mixture of embodiment 251, wherein the mRNA is inside the erythroid cell.
   253. The reaction mixture of embodiment 251 or 252, which comprises a plurality of erythroid cells.
   254. A method of assaying a reaction mixture comprising enucleated erythroid cells that comprise an exogenous protein for a ribonuclease inhibitor, comprising:
providing a reaction mixture comprising enucleated erythroid cells that comprise an exogenous protein,
assaying for the presence or level of a ribonuclease inhibitor, e.g., by ELISA, Western blot, or mass spectrometry, e.g., in an aliquot of the reaction mixture.
   255. The method of embodiment 254, further comprising comparing the level of ribonuclease inhibitor to a reference value.
   256. The method of embodiment 255, further comprising responsive to the comparison, one or more of:
classifying the population, e.g., as meeting a requirement or not meeting a requirement, e.g., wherein the requirement is met when the level of ribonuclease inhibitor is below the reference value,
classifying the population as suitable or not suitable for a subsequent processing step, e.g., when the population is suitable for a subsequent purification step when the level of ribonuclease inhibitor is above the reference value,
classifying the population as suitable or not suitable for use as a therapeutic, or
formulating or packaging the population, or an aliquot thereof, for therapeutic use, e.g., when the level of ribonuclease inhibitor is below the reference value.
   257. The reaction mixture or method of any of embodiments 220-256, wherein the ribonuclease inhibitor is RNAsin Plus, Protector RNAse Inhibitor, or Ribonuclease Inhibitor Huma.
   258. A method of making an erythroid cell comprising an mRNA that encodes an exogenous protein, comprising:
providing a reaction mixture comprising an erythroid cell and an mRNA encoding the exogenous protein, under conditions which inhibit protein degradation, e.g., by inclusion in the reaction mixture a proteasome inhibitor, and
maintaining the reaction mixture under conditions that allow uptake of the mRNA by the erythroid cell,
thereby making an erythroid cell comprising an mRNA encoding an exogenous protein.
   259. The method of embodiment 258, comprising providing a population of erythroid cells and contacting the population with the mRNA encoding the exogenous protein.
   260. The method of embodiment 258 or 259, wherein a plurality of erythroid cells of the population each takes up an mRNA encoding the exogenous protein.
   261. The method of any of embodiments 258-260, wherein the cell or plurality of cells express the exogenous protein.
   262. The method of any of embodiments 258-261, wherein the cell or plurality of cells comprise the exogenous protein.
   263. The method of any of embodiments 258-262, which further comprises electroporating the cell or population of cells.
   264. The method of any of embodiments 258-263, which further comprises contacting a population of erythroid cells with a proteasome inhibitor.
   265. The method of any of embodiments 258-264, which comprises contacting the population of cells with the proteasome inhibitor before, during, or after contacting the cells with the mRNA, e.g., 0.5-2 days before or after contacting the cells with the mRNA.
   266. The method of any of embodiments 258-265, which comprises contacting the cells with the proteasome inhibitor at day 4, 5, or 6 of maturation phase.
   267. The method of any of embodiments 258-266, wherein the cell is in maturation phase.
   268. The method of any of embodiments 258-267, which comprises contacting the cells with the proteasome inhibitor at a time when the cells comprise one or more (e.g., 2, 3, 4, 5, 6, 7, 8, 9, 10, 11, 12, 13, 14, 15, 16, 17, 18, 19, 20, 21, or more) of the following properties:
i.a) 2-40%, 3-33%, 5-30%, 10-25%, or 15-20% of the cells in the population are enucleated;
i.b) greater than 0%, 0.1%, 0.2%, or 0.5%, but less than 2%, 3%, 4%, or 5% of the cells in the population are enucleated;
i.c) greater than 0%, 0.1%, 0.2%, or 0.5%, but less than 6%, 10%, 15%, 20%, or 25% of the cells in the population are enucleated;
i.d) greater than 0%, 0.1%, 0.2%, or 0.5%, but less than 30%, 35%, 40%, 45%, or 50% of the cells in the population are enucleated;
i.e) no more than 1%, 2%, 3%, 5%, 10%, 15%, or 20% of the cells in the population are enucleated;
i.f) no more than 25%, 30%, 35%, 40%, 45%, or 50% of the cells in the population are enucleated;
i.g) the population of cells has reached 6-70%, 10-60%, 20-50%, or 30-40% of maximal enucleation;
i.h) the population of cells has reached no more than 1%, 2%, 3%, 5%, 10%, 15%, or 20% of maximal enucleation;
i.i) the population of cells has reached no more than 25%, 30%, 35%, 40%, 45%, 50%, or 60% of maximal enucleation;
ii.a) the population of cells is fewer than 3, 2, or 1 population doubling from a plateau in cell division;
ii.b) the population of cells is capable of fewer than 3, 2, or 1 population doubling;
ii.c) the population will increase by no more than 1.5, 2, or 3 fold before the population reaches an enucleation level of at least 70% of cells in the population;
iii.a) at least 80%, 85%, 90%, 95%, or 99% of the cells in the population are normoblasts (e.g., polychromatic or orthochromatic normoblasts);
iii.b) at least 50%, 60%, 70%, 75%, or 79% of the cells in the population are normoblasts (e.g., polychromatic or orthochromatic normoblasts);
iii.c) 30-90%, 40-90%, 50-90%, 60-90%, or 70-90% of the cells in the population are normoblasts (e.g., polychromatic or orthochromatic normoblasts);
iii.d) at least 80%, 85%, 90%, 95%, or 99% of the cells in the population exhibit the morphology of a normoblast (e.g., a polychromatic or orthochromatic normoblast);
iii.e) at least 50%, 60%, 70%, 75%, or 79% of the cells in the population exhibit the morphology of a normoblast (e.g., a polychromatic or orthochromatic normoblast);
iii.f) 30-90%, 40-90%, 50-90%, 60-90%, or 70-90% of the cells in the population exhibit the morphology of a normoblast (e.g., a polychromatic or orthochromatic normoblast);
iv.a) the population of cells has at least 60%, 70%, 80%, or 90% of maximal translational activity;
iv.b) the population of cells has at least 20%, 30%, 40%, or 50% of maximal translational activity;
iv.c) the population of cells has a translational activity of at least 600,000, 800,000, 1,000,000, 1,200,000, 1,400,000, 1,600,000, 1,800,000, 2,000,000, 2,200,000, or 2,400,000, as measured by a BONCAT assay, e.g., by the translation assay of Example 10; or
iv. d) the population of cells has a translational activity of 600,000-2,400,000, 800,000-2,200,000, 1,000,000-2,000,000, 1,200,000-1,800,000, or 1,400,000-1,600,000, as measured by a BONCAT assay, e.g., by the translation assay of Example 10.
   269. The method of embodiment 268, wherein the population of erythroid cells or the population of differentiating erythroid cells is a population of erythroid cells comprising a property from i and a property from ii.
   270. The method of embodiment 268, wherein the population of erythroid cells or the population of differentiating erythroid cells is a population of erythroid cells comprising a property from i and a property from iii.
   271. The method of embodiment 268, wherein the population of erythroid cells or the population of differentiating erythroid cells is a population of erythroid cells comprising a property from i and a property from iv.
   272. The method of embodiment 268, wherein the population of erythroid cells or the population of differentiating erythroid cells is a population of erythroid cells comprising a property from ii and a property from iii.
   273. The method of embodiment 268, wherein the population of erythroid cells or the population of differentiating erythroid cells is a population of erythroid cells comprising a property from ii and a property from iv.
   274. The method of embodiment 268, wherein the population of erythroid cells or the population of differentiating erythroid cells is a population of erythroid cells comprising a property from iii and a property from iv.
   275. The method of embodiment 268, wherein the population of erythroid cells or the population of differentiating erythroid cells is a population of erythroid cells comprising a property from i, a property from ii, and a property from iii.
   276. The method of embodiment 268, wherein the population of erythroid cells or the population of differentiating erythroid cells is a population of erythroid cells comprising a property from i, a property from ii, and a property from iv.
   277. The method of embodiment 268, wherein the population of erythroid cells or the population of differentiating erythroid cells is a population of erythroid cells comprising a property from i, a property from iii, and a property from iv.
   278. The method of embodiment 268, wherein the population of erythroid cells or the population of differentiating erythroid cells is a population of erythroid cells comprising a property from ii, a property from iii, and a property from iv.
   279. The method of any of embodiments 258-278, which comprises contacting the cells with the proteasome inhibitor at a time when (e.g., by a flow cytometry assay, e.g., a flow cytometry assay of Example 10) the cells comprise one or more (e.g., 2, 3, 4, 5, or more) of the following properties:
84-99%, 85-95%, or about 90% of the cells in the population are GPA-positive;
at least 84%, 85%, 90%, 95%, or 99% of the cells in the population are GPA-positive;
54-99%, 55-98%, 60-95%, 65-90%, 70-85%, or 75-80% of the cells in the population are band3-positive;
at least 54%, 55%, 60%, 65%, 70%, 75%, 80%, 85%, 90%, 95%, 98%, or 99% of the cells in the population are band3-positive;
96-100%, 97-99%, or about 98% of the cells in the population are alpha4 integrin-positive; or
at least 95%, 96%, 97%, 98%, or 99% of the cells in the population are alpha4 integrin-positive.
   280. The method of any of embodiments 258-279, wherein the mRNA is in vitro transcribed mRNA.
   281. The method of any of embodiments 258-280, wherein at least 50%, 60%, 70%, 80%, 85%, 90%, or 95% of the cells of the population are viable 5 days after the cells are contacted with the mRNA.
   282. The method of any of embodiments 258-281, wherein at least 10%, 20%, 30%, 40%, 50%, 60%, 70%, 80%, or 90% of the cells of the population are enucleated 5 days after the cells are contacted with the mRNA.
   283. The method of any of embodiments 258-282, wherein the proportion of cells that are enucleated 5 days after the cells are contacted with the mRNA is at least 50%, 60%, 70%, 80%, 90%, or 95% of the proportion of cells that are enucleated in an otherwise similar population of cells not treated with the proteasome inhibitor.
   284. The method of any of embodiments 258-283, wherein the population of cells comprises at least 1×10⁶, 2×10⁶, 5×10⁶, 1×10⁷, 2×10⁷, 5×10⁷, or 1×10⁸ cells at the time the cells are contacted with the mRNA.
   285. The method of any of embodiments 258-284, wherein the population of cells expands by at least 10%, 20%, 30%, 40%, 50%, 60%, 70%, 80%, or 90% within 5 days after the cells are contacted with the mRNA.
   286. The method of any of embodiments 258-285, wherein at least 50%, 60%, 70%, 80%, 85%, 90%, or 95% of the cells of the population express the exogenous protein, e.g., 5 days after the cells are contacted with the mRNA.
   287. The method of any of embodiments 258-286, wherein at least 50%, 60%, 70%, 80%, 85%, 90%, or 95% of the cells of the population comprise at least 1,000, 2,000, 5,000, 10,000, 20,000, 50,000, or 100,000 copies of the exogenous protein, e.g., 5 days after the cells are contacted with the mRNA.
   288. The method of any of embodiments 258-287, wherein the population of cells comprises at least 10%, 20%, 30%, 40%, 50%, 60%, 70%, 80%, or 90% more, or at least 2-fold, 3-fold, 4-fold, or 5-fold more of the exogenous protein than an otherwise similar population of cells not treated with the proteasome inhibitor.
   289. A reaction mixture comprising: i) an erythroid cell, ii) an mRNA comprising an exogenous protein and iii) a proteasome inhibitor.
   290. The reaction mixture of embodiment 289, wherein the mRNA is inside the erythroid cell.
   291. The reaction mixture of embodiment 289 or 290, which comprises a plurality of erythroid cells.
   292. A method of assaying a reaction mixture comprising enucleated erythroid cells that comprise an exogenous protein for a proteasome inhibitor, comprising:
providing a reaction mixture comprising enucleated erythroid cells that comprise an exogenous protein,
assaying for the presence or level of a proteasome inhibitor, e.g., by ELISA, Western blot, or mass spectrometry, e.g., in an aliquot of the reaction mixture.
   293. The method of embodiment 292, further comprising comparing the level of proteasome inhibitor to a reference value.
   294. The method of embodiment 293, further comprising, responsive to the comparison, one or more of:
classifying the population, e.g., as meeting a requirement or not meeting a requirement, e.g., wherein the requirement is met when the level of proteasome inhibitor is below the reference value,
classifying the population as suitable or not suitable for a subsequent processing step, e.g., when the population is suitable for a subsequent purification step when the level of proteasome inhibitor is above the reference value,
classifying the population as suitable or not suitable for use as a therapeutic, or
formulating or packaging the population, or an aliquot thereof, for therapeutic use, e.g., when the level of proteasome inhibitor is below the reference value.
   295. The reaction mixture or method of any of embodiments 258-294, wherein the proteasome inhibitor is a 20S proteasome inhibitor, e.g., MG-132 or carfilzomib, or a 26S proteasome inhibitor, e.g., bortezomib.
   296. A method of making an erythroid cell comprising an mRNA encoding a first exogenous protein and a second exogenous protein, comprising:
a) providing an erythroid cell, e.g., in maturation phase, and
b) contacting the erythroid cell with an mRNA encoding the first exogenous protein and a second mRNA encoding the second exogenous protein, under conditions that allow uptake of the first mRNA and second mRNA by the erythroid cell,
thereby making an erythroid cell comprising the first mRNA and the second mRNA.
   297. The method of embodiment 296, wherein the erythroid cell comprises at least 1,000, 2,000, 5,000, 10,000, 20,000, 50,000, or 100,000 copies of the first exogenous protein and the second exogenous protein, e.g., 5 days after the contacting with the mRNA.
   298. A method of producing a population of erythroid cells expressing a first exogenous protein and a second exogenous protein, comprising:
a) providing a population of erythroid cells, e.g., in maturation phase, and
b) contacting the population of erythroid cells with a first mRNA encoding a first protein and a second mRNA encoding a second protein,
thereby making an erythroid cell comprising an mRNA encoding an exogenous protein
wherein at least 80%, 85%, 90%, 95%, 96%, 97%, 98%, or 99% of cells in the population comprise both of the first mRNA and the second mRNA.
   299. The method of embodiment 298, wherein the population of erythroid cells comprises an average of at least 1,000, 2,000, 5,000, 10,000, 20,000, 50,000, or 100,000 copies of the first exogenous protein and the second exogenous protein per cell, e.g., 5 days after the contacting with the mRNA.
   300. The method of any of embodiments 296-299, wherein the contacting comprises performing electroporation.
   301. The method of any of embodiments 298-300, wherein the population of cells comprise the first exogenous protein and the second exogenous protein in at least 80%, 85%, 90%, 95%, 96%, 97%, 98%, or 99% of cells for at least 5 days after the cells were contacted with the first and second mRNAs.
   302. A population of erythroid cells wherein at least 80%, 85%, 90%, 95%, 96%, 97%, 98%, or 99% of cells in the population express a first exogenous protein and a second exogenous protein, wherein the population was not made by contacting the cells with DNA encoding the first or second exogenous protein.
   303. A method of producing a plurality of erythroid cells comprising a predetermined number of copies of an exogenous protein per cell, comprising contacting the population with a predetermined amount of mRNA encoding the exogenous protein, thereby making the erythroid cell comprising the predetermined amount of the exogenous protein.
   304. The method of embodiment 303, further comprising evaluating one or more of the plurality of erythroid cells (e.g., enucleated erythroid cells) to determine the amount of the exogenous protein.
   305. A method of evaluating the amount of an exogenous protein in a sample of erythroid cells, e.g., enucleated erythroid cells comprising:
providing a plurality of erythroid cells comprising a predetermined number of copies of an exogenous protein per cell, which was made by contacting the population with a predetermined amount of mRNA encoding the exogenous protein, and
determining the amount of the exogenous protein in the plurality of erythroid cells.
   306. The method of embodiment any of embodiments 303-305, wherein:
contacting the cell population with 0.6±20% ug of mRNA per 5E6 cells in the population yields a population of cells expressing 1,000,000±20% copies of the exogenous protein per cell,
contacting the cell population with 0.4±20% ug of mRNA per 5E6 cells in the population yields a population of cells expressing 870,000±20% copies of the exogenous protein per cell,
contacting the cell population with 0.2±20% ug of mRNA per 5E6 cells in the population yields a population of cells expressing 610,000±20% copies of the exogenous protein per cell,
contacting the cell population with 0.1±20% ug of mRNA per 5E6 cells in the population yields a population of cells expressing 270,000±20% copies of the exogenous protein per cell,
contacting the cell population with 0.05±20% ug of mRNA per 5E6 cells in the population yields a population of cells expressing 100,000±20% copies of the exogenous protein per cell, or
contacting the cell population with 0.025±20% ug of mRNA per 5E6 cells in the population yields a population of cells expressing 43,000±20% copies of the exogenous protein per cell.
   307. The method of any of embodiments 303-306, wherein at least 50%, 60%, 70%, 80%, 85%, 90%, or 95% of the cells of the population express the exogenous protein 1 day after the cells are contacted with the exogenous protein.

### EXAMPLES

### Example 1: Methods of Delivering Exogenous Modified or Unmodified RNA

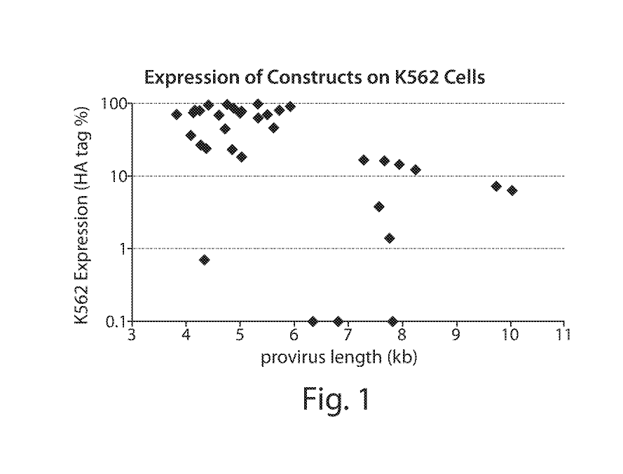

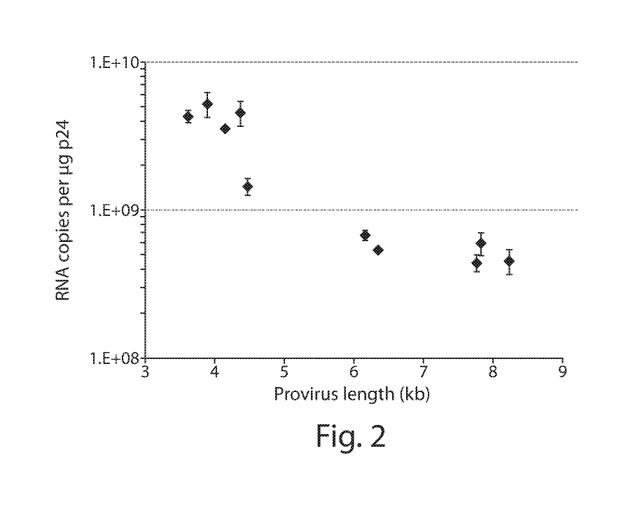

For lentivirally transduced K562 cells the expression of an epitope tag (HA tag) contained on the transgene is inversely correlated to the provirus length, with an approximately 1-log decrease in percent of cells expressing the HA tag for provirus constructs larger than approximately 6 kb (see FIG. 1). While not intending to be bound by any particular theory, it is believed that the reason for the decrease in transduction efficiency has to do with the reduced packaging efficiency of longer provirus sequences within lentiviruses. A set of lentivirus constructs of provirus lengths ranging from 3.6 kb to 8.2 kb were tested (see FIG. 2). The number of lentivirus particles produced was quantified using the number of p24 capsid proteins measured by ELISA. The number of copies of provirus RNA was measured by quantitative polymerase chain reaction (qPCR). Normalization provides a quantification of RNA copies per microgram p24 capsid protein as a function of provirus length. In the experiments, for provirus length <5 kb about 3×10⁹ RNA copies per microgram p24 could be seen. For constructs >6 kb no virus preparation exhibited more than about 8×10⁸ RNA copies per microgram p24 capsid. A size-dependent difference between RNA-containing and RNA-deficient virus preparations can be observed leading to a reduction in transduction efficiency.

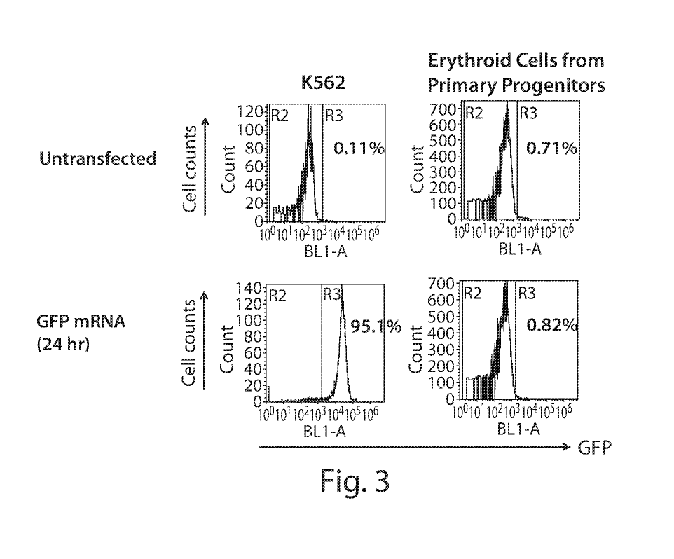

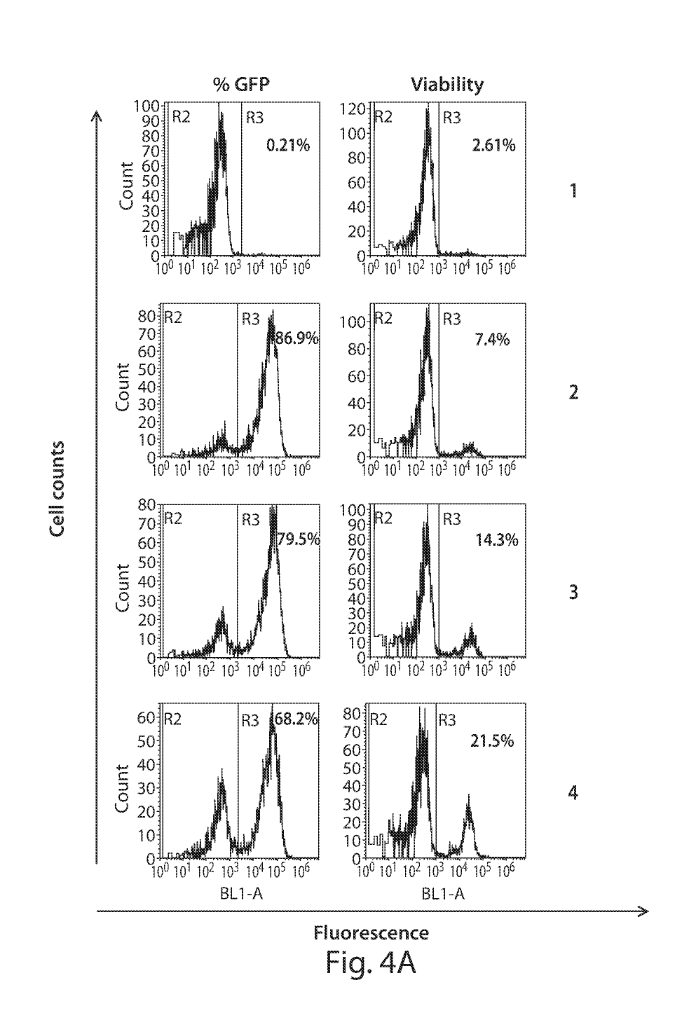

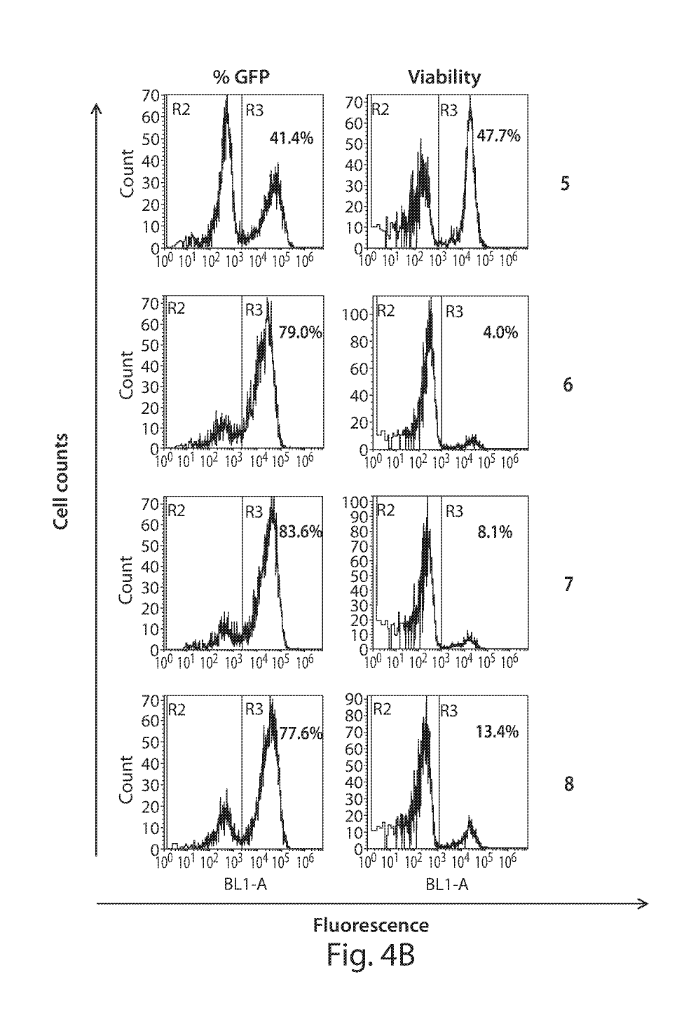

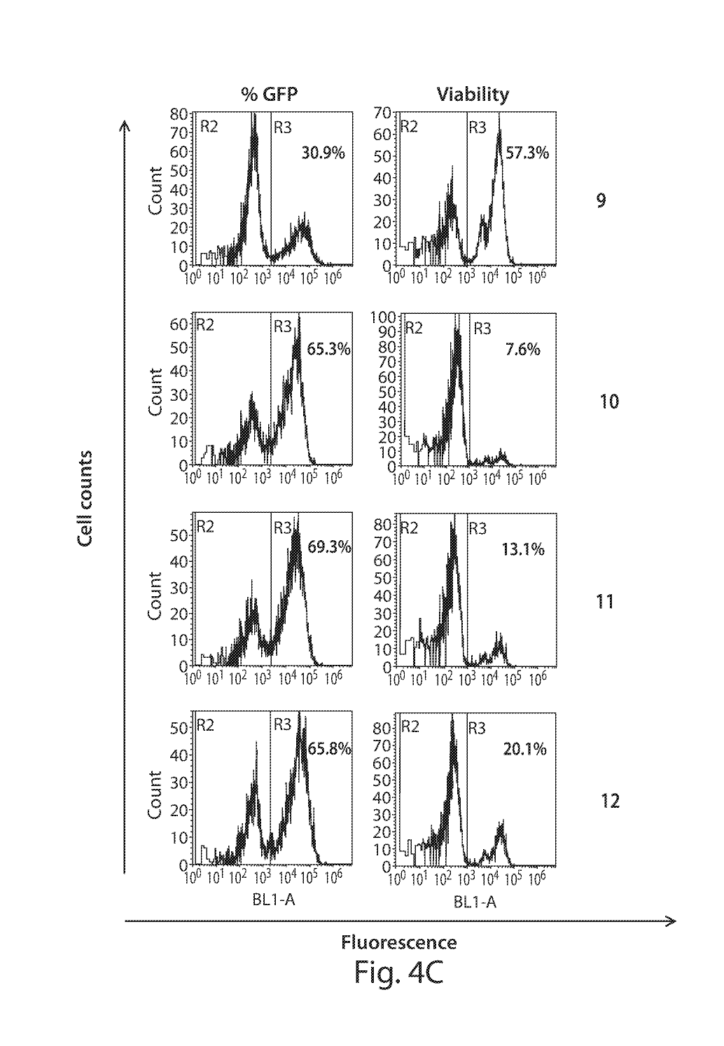

Electroporation by a single pulse of 260V/150 μF of K562 cells and cultured erythroid cells (from primary cells) with mRNA encoding for green fluorescent protein (GFP) was performed (see FIG. 3). Successful gene transfer was measured by reading the fluorescence from the GFP, which requires that the mRNA enter the cell and then be translated into protein. The conditions from the literature that lead to successful electroporation of K562 cells (Van Tendeloo et al., Blood 2001 98(1):49-56) are insufficient for the effective delivery of exogenous nucleic acids to cultured erythroid cells (derived from primary progenitors). More than 50 different conditions for electroporation of cultured erythroid cells from primary progenitors were tested. Transfection efficiencies generally ranged from 0.1% transfected cells to more than 85% transfected cells (see FIGS. 4A-4C). FIGS. 4A-4C show the translation of GFP from mRNA following electroporation of cultured erythroid cells from primary progenitors at day 8 of differentiation for 12 different conditions. Viability was measured using LIVE/DEAD stain from Life Technologies, in which cells that were negative for the stain were considered viable. Condition 1 corresponds to the untransfected control (0.21% GFP, 97.39% viability). Depending on the electroporation conditions used, cells had very good uptake of mRNA (86.9%) and high viability (92.6%), e.g. condition 2, or poor uptake of mRNA (30.9%) and poor viability (42.7%), e.g. condition 9.

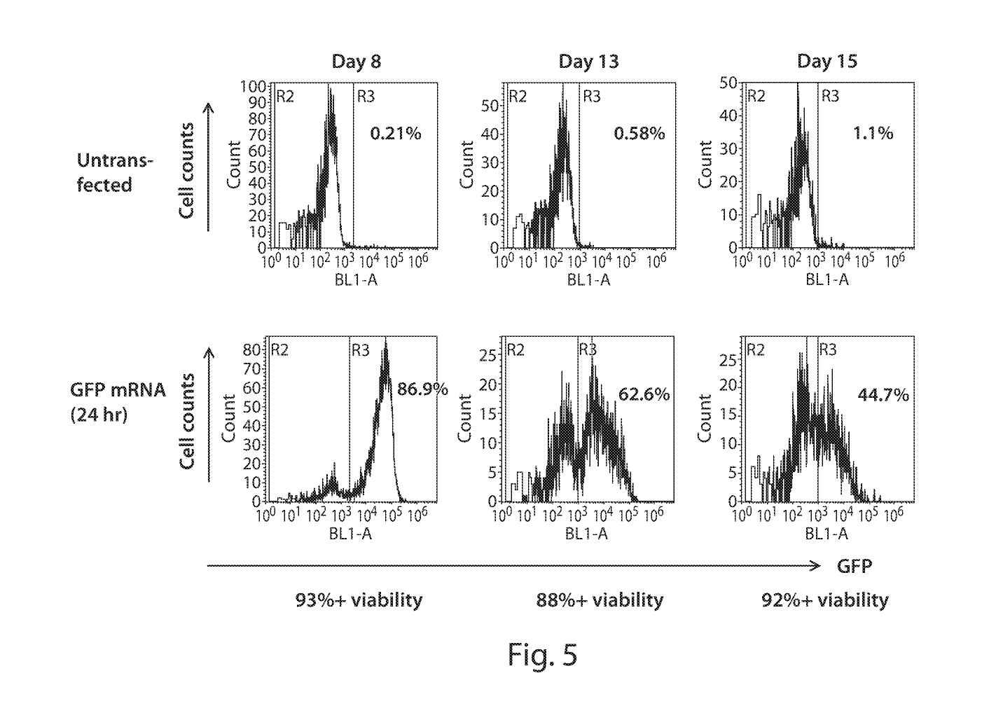

As the cells continue to differentiate, different electroporation conditions are required to achieve good transgene uptake and expression while maintaining high viability. Greater than 50 conditions were tested on cells over the course of an approximately 20 day differentiation culture to identify conditions that were conducive to good transfection and good viability. FIG. 5 shows the successful transfection of cells with GFP mRNA by electroporation at three different time points—day 8, day 13, and day 15. Suitable conditions are summarized in Tables 5 to 7. Cultured erythroid cells were also transfected with GFP mRNA by electroporation on day 10 and day 12 of differentiation, resulting in GFP expression (data not shown).

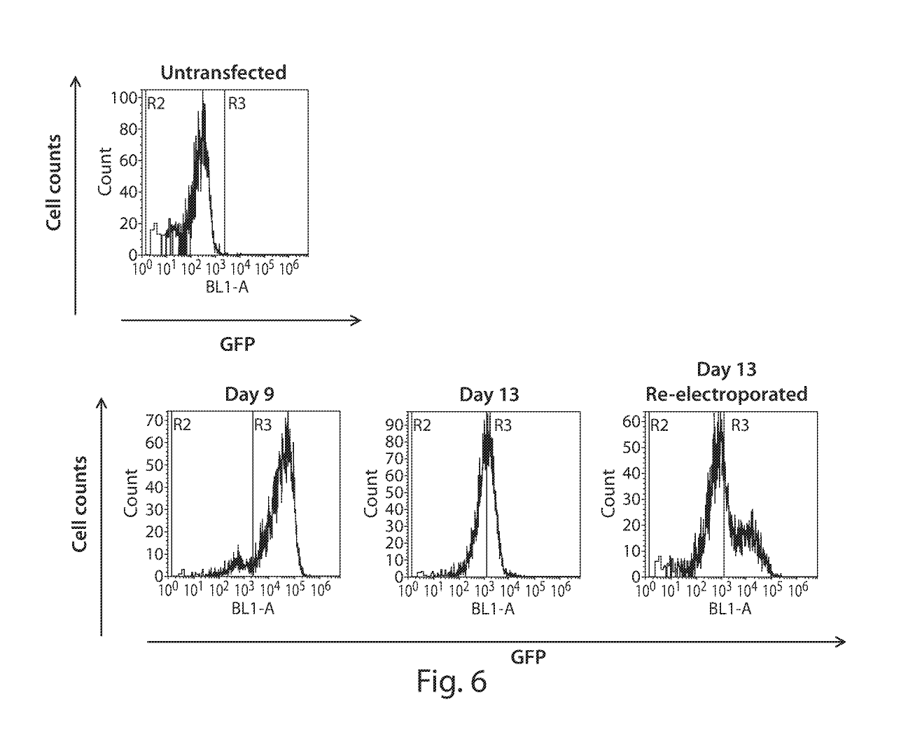

It was also observed that electroporation under the conditions disclosed herein of erythroid cells cultured from primary progenitors did not appear to damage the cells' ability to terminally differentiate. Cells that had been electroporated once were re-electroporated and again successfully took up and translated the transgene. FIG. 6 shows a population of erythroid cells cultured from primary progenitors that were electroporated at day 9, allowed to divide for four days during which the amount of GFP fluorescence decreased—likely because of dilution of the mRNA and protein through cell division—and then re-electroporated at day 13.

Cultured erythroid cells were electroporated with GFP mRNA on day 4 of differentiation, which is during the expansion phase where the cells are relatively undifferentiated. On day 8 of differentiation, the cells showed GFP fluorescence and high viability by 7AAD staining, as shown in Table 8. In Table 8, P1 indicates the percentage of the main population that constitutes cells (e.g., high P1 values mean low levels of debris); % GFP indicates the percent of cells in P1 that show GFP fluorescence, MFI is the mean fluorescent intensity of the GFP+ cells, and % AAD-indicates the percent of cells that are AAD negative, where viable cells are AAD negative.

### Example 2: ELISA

P24 protein was quantified using a commercial kit (Clontech) following manufacturer's protocol. Briefly, viral supernatants were dispensed into tubes with 20 uL lysis buffer and incubated at 37 C for 60 minutes, then transferred to a microtiter plate. The microtiter plate was washed and incubated with 100 μl of Anti-p24 (Biotin conjugate) detector antibody at 37 C for 60 minutes. Following a wash, the plate was incubated with 100 μl of Streptavidin-HRP conjugate at room temperature for 30 minutes, then washed again. 12. Substrate Solution was added to the plate and incubated at room temperature (18-25° C.) for 20 (±2) minutes. The reaction was topped with stop solution, and colorimetric readout detected by absorbance at 450 nm.

### Example 3: qPCR

Viral RNA copies were quantified using a commercial lentivector qRT-PCR kit (Clontech) following manufacturer's protocol. Briefly, an RNA virus purification kit was used to extract RNA from lentiviral supernatant. The PCR reaction was performed with standard lentivirus primers (forward and reverse) that recognize conserved sequences on the viral genome and are not dependent on the specific transgene encoded by the vector. The RT reaction was performed with a 42 C 5 min incubation followed by a 95° C. 10 sec incubation, followed by 40 cycles of 95 C for 5 sec and 60 C for 30 sec. The instrument used was a Life Technologies QuantStudio.

### Example 4: Production of mRNA by In Vitro Transcription

Kits for in vitro production of mRNA are available commercially, e.g., from Life Technologies MAxiscript T7 kit. Briefly, a gene of interest is cloned into an appropriate T7 promoter-containing plasmid DNA by standard molecular biology techniques. The transcription reaction is set up with 1 ug DNA template, 2 uL 10×transcription buffer, 1 uL each of 10 mM ATP, CTP, GTP, and UTP, 2 uL of T7 polymerase enzyme mix, in a total volume of 20 uL. The reaction is mixed thoroughly and incubated for 1 hr at 37 C. To remove contaminating residual plasmid DNA, 1 uL turbo DNAse is added and the reaction incubated for 15 minutes at 37 C. The reaction is stopped by the addition of 1 uL 0.5 M EDTA. The transcript is purified by gel electrophoresis or spin column purification.

### Example 5: Electroporation

Cells are washed in RPMI buffer, loaded into a Life Technologies Neon electroporation instrument at a density of 1×10̂7 cells/mL in a total volume of 10 uL, and electroporated with the following conditions: 1 pulse of 1000 V, 50 ms pulse width.

### Example 6: Electroporation with Chemically Modified mRNA

Chemically modified mRNA encoding GFP was purchased from TriLink. The RNA contains pseudo-uridine and 5-methyl cytosine. Differentiating erythroid cells were electroporated at day 4, 8, 10, or 12 of differentiation. On all days of differentiation tested, and under different electroporation conditions tested, GFP fluorescence was observed. Table 9 indicates the GFP fluorescence levels observed when cells were electroporated on day 4 and observed on day 8. Table 10 indicates GFP fluorescence levels observed when cells were electroporated on day 12 and observed on day 15. GFP fluorescence was also observed in cells electroporated at day 8 or 10 of differentiation (data not shown).

Cell viability and proliferation ability were measured in electroporated cells, using trypan blue staining. The cells were electroporated at day 8 of differentiation with unmodified GFP mRNA or TriLink chemically modified RNA comprising pseudo-uridine and 5-methyl cytosine. On day 9, GFP fluorescence was observed in the cells receiving unmodified or modified RNA (data not shown). Also on day 9, the total number of cells, number of live cells, and cell viability were measured. In the samples electroporated with unmodified mRNA, the number of live cells was lower than the number of live cells in the control cells that were electroporated without adding exogenous nucleic acid (see Table 11). This decline was partially reversed when modified RNA was used (Table 11). This indicates that electroporation with unmodified RNA may reduce cell growth or viability, and use of modified RNA can at least partially rescue growth or viability.

### Example 7: Heterologous Untranslated Regions

Erythroid cells were electroporated with in vitro transcribed, GFP mRNA having a hemoglobin 3′ UTR sequence appended (“Hemo-GFP”). The mRNA was not chemically modified. The cells were then assayed for GFP fluorescence by flow cytometry two days after electroporation. 59.7% of the cells were GFP-positive. The mean fluorescence intensity of the GFP-positive cells was 35069 units.

### Example 8: mRNA Electroporation During Maturation Phase

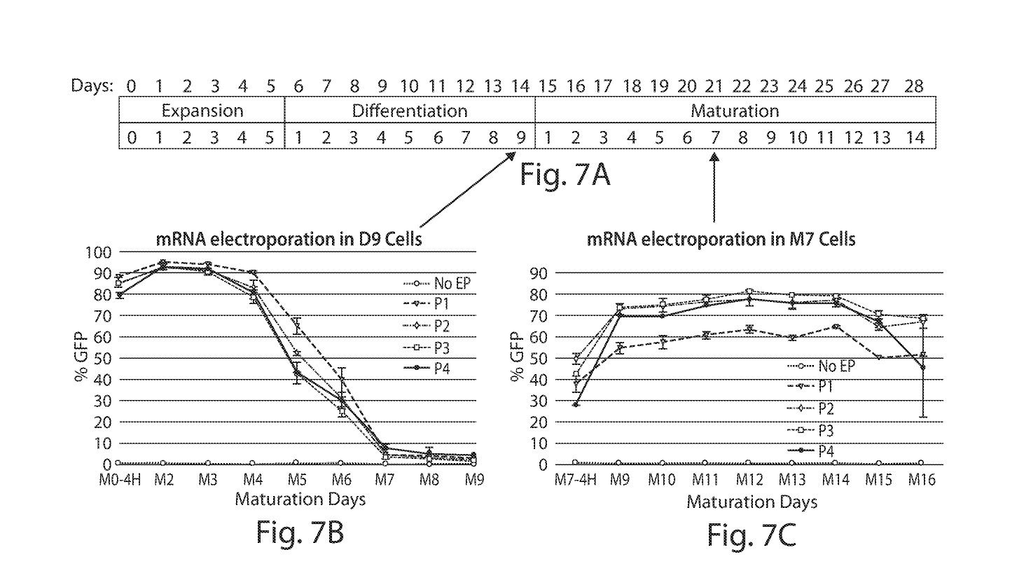

As illustrated in FIG. 7A, red blood cell differentiation can be divided into three phases: expansion (days 0-5 of expansion, which correspond to days 0-5 overall), differentiation (days 1-9 of differentiation, which correspond to days 6-14 overall), and maturation (days 1-14 of maturation, which correspond to days 15-28 overall). Expansion describes the phase of hematopoietic progenitor cell isolation and expansion in a non-differentiating environment, in order to amplify early stage cultures to meet clinical dose requirements. Differentiation describes the use of growth factors and media additives to induce erythropoiesis and specialize for red blood cell function. Maturation refers to a final stage in which red blood cells first lose their nucleus and subsequently their mitochondria and ribosome content. The mature red blood cell does not have the capacity for new mRNA synthesis or protein translation.

Red blood cell differentiation was performed in vitro, and the cells were electroporated with GFP mRNA at different timepoints. When the cells were electroporated at differentiation day 9 (overall day 14), GFP expression was observed initially but declined over the course of the 9-day experiment (FIG. 7B). When cells were electroporated on maturation day 7 (overall day 21), GFP expression was prolonged throughout the course of the 9-day experiment (FIG. 7C). Under four different electroporation protocols (P1-P4) the result was similar, indicating that this effect is relatively independent of electroporation conditions.

It was surprising that electroporation at such late stages worked as well as it did. As cited by Steinberg (Steinberg, M., Disorders of Hemoglobin: Genetics, Pathophysiology, and Clinical Management, Cambridge University Press, 2001) The adult red cell is organized to carry the synthesized hemoglobin for its role in gaseous transport; the nucleus, the capacity for protein synthesis, and the ability to diversify its function have been cast off for the ultimate purpose of hemoglobin transport via biologically economical means.” The art generally regarded maturation as a phase when erythroid cells are enucleated and shed ribosomes and mitochondria. Shedding ribosomes leads to the expectation that maturation phase erythroid cells translate poorly and therefore should be incapable of new protein synthesis.

Thus, it was surprising that maturation phase erythroid cells could translate a transgenic mRNA at least as well as a differentiation phase erythroid cell, and even more surprising that the maturation phase erythroid cell produced more sustained level of transgenic protein than the differentiation phase erythroid cell. This identifies a unique stage of erythroid development, contrary to traditional models, in which new protein synthesis from exogenously provided RNA can be achieved in enucleated red blood cells. This identifies hitherto unknown pathways for Achieving Stable Protein Production in Late Stage Red Blood Cell Products.

### Example 9: Timing of Electroporation

Several different timepoints were tested for electroporating an mRNA encoding a reporter protein (GFP) into a population of erythroid cells under maturation conditions. Specifically, electroporation was tested at days 4, 5, 6, and 7 of maturation. The cells were assayed for GFP expression by flow cytometry at every 24 hours for at least 6 days after electroporation. Suitable electroporation conditions are described, e.g., in Example 1 herein and in International Application WO2016/183482, which is herein incorporated by reference in its entirety.

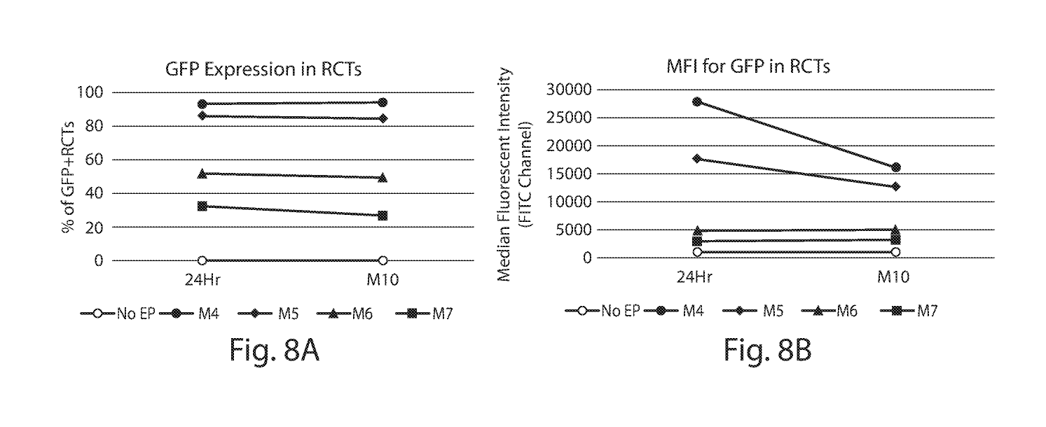

As shown in FIG. 8A, cells electroporated at all timepoints gave prolonged GFP expression. However, cells electroporated at days M4 and M5 gave a higher percentage of cells expressing GFP than cells electroporated at M6 or M7. This experiment indicates a window of erythroid cell maturation that is particularly amenable to expression of a transgene. FIG. 8B shows that GFP levels in the population decline somewhat in cells transfected at M4 or M5 over the time course; however GFP expression in these cells is still higher than that in control cells and cells electroporated at later timepoints.

While not wishing to be bound by theory, the window may indicate a timepoint that is early in maturation enough that the cell's translation machinery has not yet been lost, while simultaneously being late enough in maturation that the exogenous mRNA and encoded protein do not get unduly diluted by subsequent cell division. This window was further characterized as described in Example 10.

### Example 10: Characteristics of Maturing Erythroid Cells

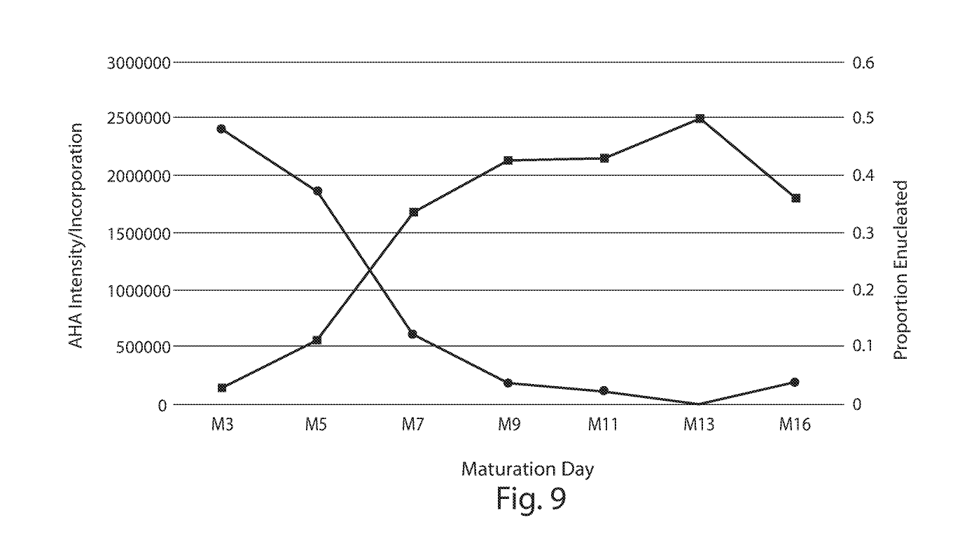

Next, maturing erythroid cells were characterized at several timepoints for their translation activity and enucleation level. Translational activity was measured by biorthogonal noncanonical amino acid tagging, or BONCAT. Suitable BONCAT assays are described, e.g., in Hatzenpichler et al., “In situ visualization of newly synthesized proteins in environmental microbes using amino acid tagging and click chemistry” Environmental Microbiology (2014) 16(8), 2568-2590. This assay is based on the in vivo incorporation of a surrogate for L-methionine, the non-canonical amino acid L-azidohomoalanine (AHA), following fluorescent labeling of incorporated AHA cellular proteins by Click Chemistry. The protocol has been modified and optimized for mammalian primary cells particularly human erythroid progenitors by increasing the AHA concentration from 1 mM to 2 mM, optimized the incubation time to 3 h, and dibenzocyclooctyne group (DBCO) has been used which allows Copper-free Click Chemistry to be analyzed by gel electrophoresis and infrared imaging. A population of erythroid cells was exposed to expansion, differentiation, and maturation conditions, and samples of 3×10⁶ cells were collected on days M3, M5, M7, M9, M11, M15, and M16. As shown in FIG. 9, translational activity of the cells declined dramatically over the time course, indicating that the erythroid cells were losing translation machinery. Over the same time course, the proportion of enucleated cells in the population rose dramatically.

Cell surface markers were also assayed in erythroid cells at different stages of maturation, by flow cytometry. As shown in Table 13, the percentage of GPA-positive cells and Band3-positive cells rose from during maturation, and the percentage of Alpha4 integrin-positive remained high throughout the time course.

### Example 11: Ribonuclease Inhibitors Increase Protein Expression in Electroporated Erythroid Cells

This experiment demonstrates that exposing erythroid cells to ribonuclease inhibitors increases expression of a transgene.

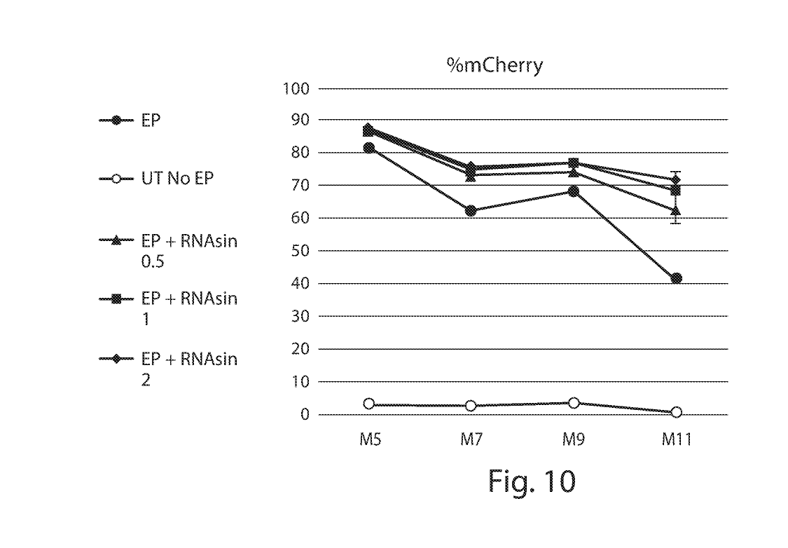

Erythroid cells were differentiated, exposed to maturation conditions, and electroporated at day M4 with mRNA encoding a reporter gene (mCherry). 2×10⁶ cells were treated with RNasin before the mRNA was added to the cells at a level of 0.5 U/uL, 1 U/uL, or 2 U/ul, or no RNasin as a control. A non-electroporated control was also included. The cells were assayed at days M5, M7, M9, and M11. As shown in FIG. 10, the percentage of cells expressing mCherry was higher in cells treated with RNasin than in cells without RNasin, especially at the M11 timepoint. RNasin treatment did not negatively impact cell viability or enucleation (data not shown).

### Example 12: Proteasome Inhibitors Increase Protein Expression in Electroporated Erythroid Cells

This experiment demonstrates that exposing erythroid cells to protease inhibitors increases expression of a transgene.

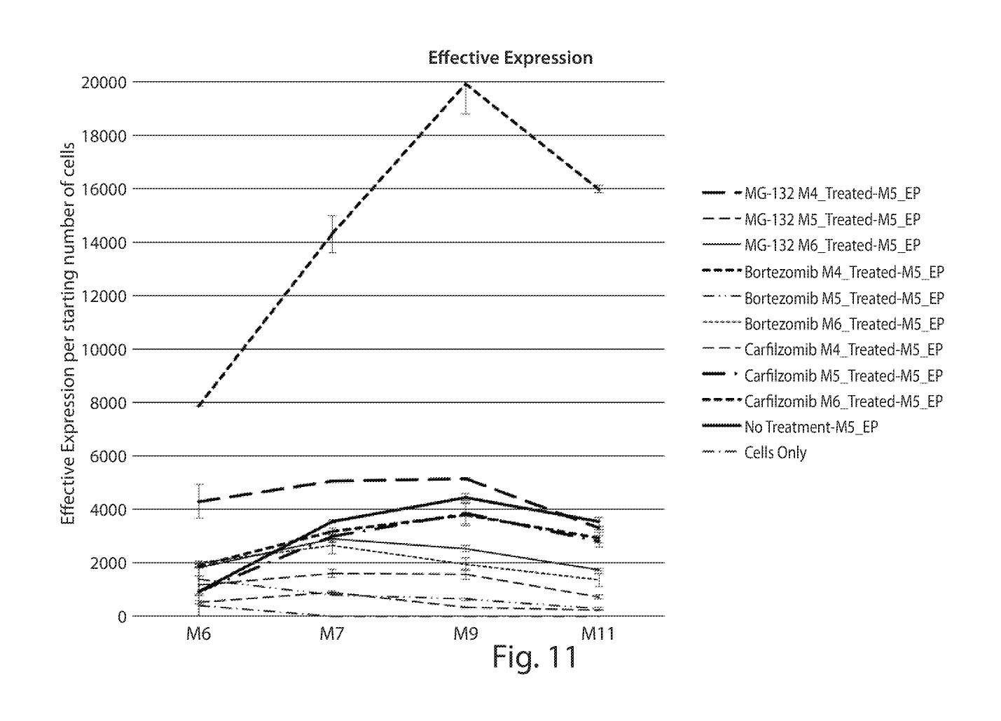

Erythroid cells were differentiated, exposed to maturation conditions, and electroporated at day M5 with mRNA encoding a reporter gene (GFP). Cells were treated with a proteasome inhibitor selected from MG-132, bortezomib, and carfilzomib, at day M4, M5, or M6. All cell samples resulted in a high percentage of GFP-positive cells, over 75%, when assayed at M7, M9, and M11 (data not shown). As shown in FIG. 11, treatment with the 20S proteasome inhibitors, MG-132 or bortezomib, before electroporation, resulted in increased effective expression of GFP at one or more timepoints. The bortezomib treatment resulted in a 4-fold increase in effective expression of GFP compared to cells not treated with a proteasome inhibitor. Treatment with the 20S proteasome inhibitors before electroporation also resulted in normal enucleation (data not Shown).

### Example 13: Co-Expression of Two or More RNAs

This Example demonstrates co-expression of two or more mRNAs in erythroid cells.

First, erythroid cells were electroporated at day M5 with EGFP mRNA alone (Table 14, first data column), mCherry mRNA alone (Table 14, second data column), or both mRNAs (Table 14, third data column). EGFP and mCherry fluorescence was assayed by flow cytometry on days M6, M11, M18, and M18. The percentage of cells expressing both). EGFP and mCherry was consistently high across timepoints (66.05%-86.55%) and comparable to the percent of cells fluorescing after electroporation with just one of the mRNAs. This experiment indicates that it is possible to achieve uniform expression of two mRNAs simultaneously.

Expression levels were also assayed. At day M13, the effective expression of mCherry in cells electroporated with mCherry mRNA only was 117, and the effective expression of mCherry in cells electroporated with both mCherry mRNA and EGFP mRNA was 85. The effective expression of EGFP in cells electroporated with EGFP mRNA only was 219, and the effective expression of EGFP in cells electroporated with both mCherry mRNA and EGFP mRNA was 201. Thus, expression levels were similar in cells electroporated with one or two mRNAs.

Viability was no lower in cells co-electroporated with two mRNAs than in cells electroporated with either mRNA alone (data not shown).

Another pair of mRNAs, encoding HA-tagged m4-1BBL and FLAG-tagged Avelumab were co-expressed in erythroid cells. RNA was added to 25×10⁶ erythroid cells at differentiation day 6 (D6) or differentiation day 7 (D7), at a concentration of 0.6 or 0.8 mg/ml mRNA. The exogenous proteins were detected by flow cytometry using an anti-HA antibody and an anti-FLAG antibody. As shown in Table 15, co-expression of the proteins was achieved in 58.5% of cells, a number comparable to the number of cells that expressed either protein alone in samples electroporated with only one of the mRNAs.

### Example 14: Dose-Expression Studies

This experiment demonstrates that a predetermined amount of an exogenous protein can be produced by contacting a population of erythroid cells with a predetermined amount of mRNA encoding the exogenous protein.

5×10⁶ cells at day 4 of maturation were contacted with different amounts of mRNA (between 0.0025 and 0.6 ug RNA per sample) and electroporated. Protein expression was assayed 24 hours after electroporation. The exogenous protein was quantified by flow cytometry using an anti-HA antibody. The average number of proteins per cell was calculated and is shown in Table 16. The percent of cells expressing the exogenous protein is also shown in Table 16. Notably, at all mRNA levels tested, the percent of cells expressing the exogenous protein is high. However, the number of copies per cell rises roughly linearly with the amount of mRNA used. Thus, the amount of protein expression desired can be obtained by selecting an appropriate of mRNA, while maintaining uniform expression across the population of cells.

### Example 15: Expression from Modified RNAs

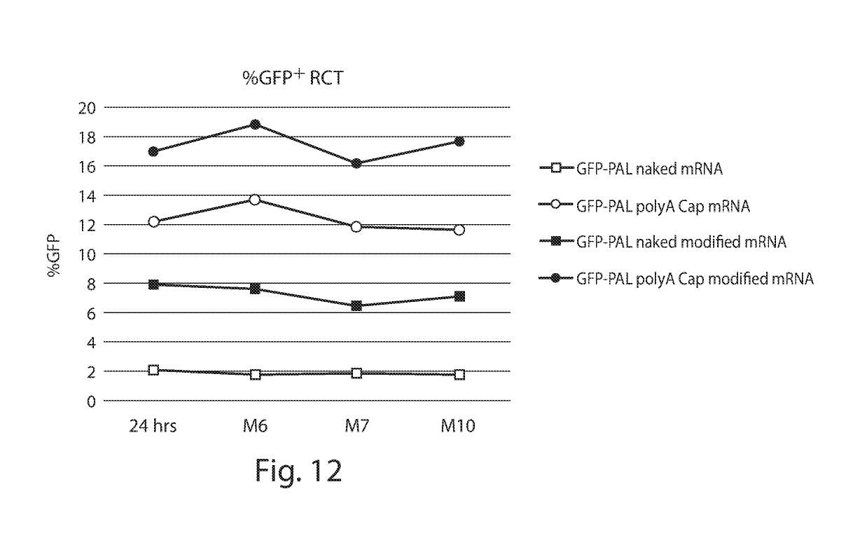

Modified mRNA was produced, comprising one or more of a 5′ cap (ARCA), polyA tail, and pseudouridine. The mRNA comprises an IRES to promote translation, an HA-encoding region to facilitate detection, and a region encoding a fusion of GFP and PAL (phenylalanine ammonia lyase). The mRNA was introduced into erythroid cells by electroporation at day M4 and was analyzed at days M5 (24 hours later), M6, M7, and M10. GFP expression was measured by flow cytometry. As shown in FIG. 12, cells expressing pseudouridine mRNA had a higher percentage of GFP-positive cells than cells expressing completely unmodified RNA. Addition of a polyA tail and cap increased the percentage of GFP-positive cells further. Finally, the percentage of cells showing expression of the GFP reporter was highest in the cells contacted with mRNA having a cap, poly-A tail, and pseudouridine incorporation.

### Tables

TABLE 1
Modified nucleotides

| | |
|---|---|
| 5-aza-uridine | N2-methyl-6-thio-guanosine |
| 2-thio-5-aza-midine | N2,N2-dimethyl-6-thio-guanosine |
| 2-thiouridine | pyridin-4-one ribonucleoside |
| 4-thio-pseudouridine | 2-thio-5-aza-uridine |
| 2-thio-pseudouridine | 2-thiomidine |
| 5-hydroxyuridine | 4-thio-pseudomidine |
| 3-methyluridine | 2-thio-pseudowidine |
| 5-carboxymethyl-uridine | 3-methylmidine |
| 1-carboxymethyl-pseudouridine | 1-propynyl-pseudomidine |
| 5-propynyl-uridine | 1-methyl-1-deaza-pseudomidine |
| 1-propynyl-pseudouridine | 2-thio-1-methyl-1-deaza-pseudouridine |
| 5-taurinomethyluridine | 4-methoxy-pseudomidine |
| 1-taurinomethyl-pseudouridine | 5′-O-(1-Thiophosphate)-Adenosine |
| 5-taurinomethyl-2-thio-uridine | 5′-O-(1-Thiophosphate)-Cytidine |
| 1-taurinomethyl-4-thio-uridine | 5′-O-(1-thiophosphate)-Guanosine |
| 5-methyl-uridine | 5′-O-(1-Thiophophate)-Uridine |
| 1-methyl-pseudouridine | 5′-O-(1-Thiophosphate)-Pseudouridine |
| 4-thio-1-methyl-pseudouridine | 2′-O-methyl-Adenosine |
| 2-thio-1-methyl-pseudouridine | 2′-O-methyl-Cytidine |
| 1-methyl-1-deaza-pseudouridine | 2′-O-methyl-Guanosine |
| 2-thio-1-methyl-1-deaza-pseudomidine | 2′-O-methyl-Uridine |
| dihydrouridine | 2′-O-methyl-Pseudouridine |
| dihydropseudouridine | 2′-O-methyl-Inosine |
| 2-thio-dihydromidine | 2-methyladenosine |
| 2-thio-dihydropseudouridine | 2-methylthio-N6-methyladenosine |
| 2-methoxyuridine | 2-methylthio-N6 isopentenyladenosine |
| 2-methoxy-4-thio-uridine | 2-methylthio-N6-(cis- |
| 4-methoxy-pseudouridine | hydroxyisopentenyl)adenosine |
| 4-methoxy-2-thio-pseudouridine | N6-methyl-N6-threonylcarbamoyladenosine |
| 5-aza-cytidine | N6-hydroxynorvalylcarbamoyladenosine |
| pseudoisocytidine | 2-methylthio-N6-hydroxynorvalyl |
| 3-methyl-cytidine | carbamoyladenosine |
| N4-acetylcytidine | 2′-O-ribosyladenosine (phosphate) |
| 5-formylcytidine | 1,2′-O-dimethylinosine |
| N4-methylcytidine | 5,2′-O-dimethylcytidine |
| 5-hydroxymethylcytidine | N4-acetyl-2′-O-methylcytidine |
| 1-methyl-pseudoisocytidine | Lysidine |
| pyrrolo-cytidine | 7-methylguanosine |
| pyrrolo-pseudoisocytidine | N2,2′-O-dimethylguanosine |
| 2-thio-cytidine | N2,N2,2′-O-trimethylguanosine |
| 2-thio-5-methyl-cytidine | 2′-O-ribosylguanosine (phosphate) |
| 4-thio-pseudoisocytidine | Wybutosine |
| 4-thio-1-methyl-pseudoisocytidine | Peroxywybutosine |
| 4-thio-1-methyl-1-deaza-pseudoisocytidine | Hydroxywybutosine |
| 1-methyl-1-deaza-pseudoisocytidine | undermodified hydroxywybutosine |
| zebularine | methylwyosine |
| 5-aza-zebularine | queuosine |
| 5-methyl-zebularine | epoxyqueuosine |
| 5-aza-2-thio-zebularine | galactosyl-queuosine |
| 2-thio-zebularine | mannosyl-queuosine |
| 2-methoxy-cytidine | 7-cyano-7-deazaguanosine |
| 2-methoxy-5-methyl-cytidine | 7-aminomethyl-7-deazaguanosine |
| 4-methoxy-pseudoisocytidine | archaeosine |
| 4-methoxy-1-methyl-pseudoisocytidine | 5,2′-O-dimethyluridine |
| 2-aminopurine | 4-thiouridine |
| 2,6-diaminopurine | 5-methyl-2-thiouridine |
| 7-deaza-adenine | 2-thio-2′-O-methyluridine |
| 7-deaza-8-aza-adenine | 3-(3-amino-3-carboxypropyl)uridine |
| 7-deaza-2-aminopurine | 5-methoxyuridine |
| 7-deaza-8-aza-2-aminopurine | uridine 5-oxyacetic acid |
| 7-deaza-2,6-diaminopurine | uridine 5-oxyacetic acid methyl ester |
| 7-deaza-8-aza-2,6-diarninopurine | 5-(carboxyhydroxymethyl)uridine) |
| 1-methyladenosine | 5-(carboxyhydroxymethyl)uridine methyl ester |
| N6-isopentenyladenosine | 5-methoxycarbonylmethyluridine |
| N6-(cis-hydroxyisopentenyl)adenosine | 5-methoxycarbonylmethyl-2′-O-methyluridine |
| 2-methylthio-N6-(cis-hydroxyisopentenyl) | 5-methoxycarbonylmethyl-2-thiouridine |
| adenosine | 5-aminomethyl-2-thiouridine |
| N6-glycinylcarbamoyladenosine | 5-methylaminomethyluridine |
| N6-threonylcarbamoyladenosine | 5-methylaminomethyl-2-thiouridine |
| 2-methylthio-N6-threonyl | 5-methylaminomethyl-2-selenouridine |
| carbamoyladenosine | 5-carbamoylmethyluridine |
| N6,N6-dimethyladenosine | 5-carbamoylmethyl-2′-O-methyluridine |
| 7-methyladenine | 5-carboxymethylaminomethyluridine |
| 2-methylthio-adenine | 5-carboxymethylaminomethyl-2′-O- |
| 2-methoxy-adenine | methyluridine |
| inosine | 5-carboxymethylaminomethyl-2-thiouridine |
| 1-methyl-inosine | N4,2′-O-dimethylcytidine |
| wyosine | 5-carboxymethyluridine |
| wybutosine | N6,2′-O-dimethyladenosine |
| 7-deaza-guanosine | N,N6,O-2′-trimethyladenosine |
| 7-deaza-8-aza-guanosine | N2,7-dimethylguanosine |
| 6-thio-guanosine | N2,N2,7-trimethylguanosine |
| 6-thio-7-deaza-guanosine | 3,2′-O-dimethyluridine |
| 6-thio-7-deaza-8-aza-guanosine | 5-methyldihydrouridine |
| 7-methyl-guanosine | 5-formyl-2′-O-methylcytidine |
| 6-thio-7-methyl-guanosine | 1,2′-O-dimethylguanosine |
| 7-methylinosine | 4-demethylwyosine |
| 6-methoxy-guanosine | Isowyosine |
| 1-methylguanosine | N6-acetyladenosine |
| N2-methylguanosine | |
| N2,N2-dimethylguanosine | |
| 8-oxo-guanosine | |
| 7-methyl-8-oxo-guanosine | |
| 1-methyl-6-thio-guanosine | |

TABLE 2
Backbone modifications

2′-O-Methyl backbone
Peptide Nucleic Acid (PNA) backbone
phosphorothioate backbone
morpholino backbone
carbamate backbone
siloxane backbone
sulfide backbone
sulfoxide backbone
sulfone backbone
formacetyl backbone
thioformacetyl backbone
methyleneformacetyl backbone
riboacetyl backbone
alkene containing backbone
sulfamate backbone
sulfonate backbone
sulfonamide backbone
methyleneimino backbone
methylenehydrazino backbone
amide backbone

TABLE 3
Modified caps

m7GpppA
m7GpppC
m2,7GpppG
m2,2,7GpppG
m7Gpppm7G
m7,2′OmeGpppG
m72′dGpppG
m7,3′OmeGpppG
m7,3′dGpppG
GppppG
m7GppppG
m7GppppA
m7GppppC
m2,7GppppG
m2,2,7GppppG
m7Gppppm7G
m7,2′OmeGppppG
m72′dGppppG
m7,3′OmeGppppG
m7,3′dGppppG

TABLE 4
Selected Diseases, Receivers and Targets

| Category | Disease | Exogenous polypeptide | Target |
|---|---|---|---|
| Amyloidoses | AA Amyloidosis | an antibody-like binder to serum | Serum amyloid A |
| | | amyloid A protein or serum | protein and amyloid |
| | | amyloid P component | placques |
| Amyloidoses | beta2 miFcroglobulin | an antibody-like binder to beta-2 | Beta2 microglobulin or |
| | amyloidosis | microglobulin or serum amyloid | amyloid placques |
| | | P component | |
| Amyloidoses | Light chain amyloidosis | an antibody-like binder to light | Antibody light chain or |
| | | chain, serum amyloid P | amyloid placques |
| | | component | |
| Cell clearance | Cancer | an antibody-like binder to CD44 | a circulating tumor cell |
| Cell clearance | Cancer | an antibody-like binder to | a circulating tumor cell |
| | | EpCam | |
| Cell clearance | Cancer | an antibody-like binder to Her2 | a circulating tumor cell |
| Cell clearance | Cancer | an antibody-like binder to EGFR | a circulating tumor cell |
| Cell clearance | Cancer (B cell) | an antibody-like binder to CD20 | a cancerous B cell |
| Cell clearance | Cancer (B cell) | an antibody-like binder to CD19 | a cancerous B cell |
| Clearance Ab | Antiphospholipid | beta2-glycoprotein-1 | pathogenic self- |
| | syndrome | | antibody against beta2- |
| | | | glycoprotein-1 |
| Clearance Ab | Catastrophic | beta2-glycoprotein-1 | pathogenic self- |
| | antiphospholipid | | antibody against beta2- |
| | syndrome | | glycoprotein-1 |
| Clearance Ab | Cold agglutinin disease | I/i antigen | Pathogenic self- |
| | | | antibody against I/i |
| | | | antigen |
| Clearance Ab | Goodpasture syndrome | a3 NC1 domain of collagen (IV) | pathogenic self- |
| | | | antibody against a3 |
| | | | NC1 domain of |
| | | | Collagen (IV) |
| Clearance Ab | Immune | Platelet Glycoproteins (Ib-IX, | pathogenic self- |
| | thrombocytopenia | IIb-IIIa, IV, Ia-IIa) | antibody against platelet |
| | purpura | | glycoprotein |
| Clearance Ab | Membranous | Phospholipase A2 receptor | pathogenic self- |
| | Nephropathy | | antibody against |
| | | | phospholipase A2 |
| | | | receptor |
| Clearance Ab | Warm antibody hemolytic | Glycophorin A, glycophorin B, | pathogenic self- |
| | anemia | and/or glycophorin C, Rh | antibody against |
| | | antigen | glycophorins and/or Rh |
| | | | antigen |
| Complement | Age-related macular | a suitable complement regulatory | active complement |
| | degeneration | protein | |
| Complement | Atypical hemolytic | complement factor H, or a | active complement |
| | uremic syndrome | suitable complement regulatory | |
| | | protein | |
| Complement | Autoimmune hemolytic | a suitable complement regulatory | active complement |
| | anemia | molecule | |
| Complement | Complement Factor I | Complement factor I, a suitable | active complement |
| | deficiency | complement regulatory protein | |
| Complement | Non-alcoholic | a suitable complement regulatory | active complement |
| | steatohepatitis | molecule | |
| Complement | Paroxysmal nocturnal | a suitable complement regulatory | active complement |
| | hemoglobinuria | protein | |
| Enzyme | 3-methylcrotonyl-CoA | 3-methylcrotonyl-CoA | 3- |
| | carboxylase deficiency | carboxylase | hydroxyvalerylcarnitine, |
| | | | 3- |
| | | | methylcrotonylglycine |
| | | | (3-MCG) and 3- |
| | | | hydroxyisovaleric acid |
| | | | (3-HIVA) |
| Enzyme | Acute Intermittent | Porphobilinogen deaminase | Porphobilinogen |
| | Porphyria | | |
| Enzyme | Acute lymphoblastic | Asparaginase | Asparagine |
| | leukemia | | |
| Enzyme | Acute lymphocytic | Asparaginase | Asparagine |
| | leukemia, acute myeloid | | |
| | leukemia | | |
| Enzyme | Acute myeloblastic | Asparaginase | Asparagine |
| | leukemia | | |
| Enzyme | Adenine | adenine | Insoluble purine 2,8- |
| | phosphoribosyltransferase | phosphoribosyltransferase | dihydroxyadenine |
| | deficiency | | |
| Enzyme | Adenosine deaminase | Adenosine deaminase | Adenosine |
| | deficiency | | |
| Enzyme | Afibrinogenomia | FI | enzyme replacement |
| Enzyme | Alcohol poisoning | Alcohol dehydrogenase/oxidase | Ethanol |
| Enzyme | Alexander's disease | FVII | enzyme replacement |
| Enzyme | Alkaptonuria | homogentisate oxidase | homogentisate |
| Enzyme | Argininemia | Ammonia monooxygenase | ammonia |
| Enzyme | argininosuccinate aciduria | Ammonia monooxygenase | ammonia |
| Enzyme | citrullinemia type I | Ammonia monooxygenase | ammonia |
| Enzyme | Citrullinemia type II | Ammonia monooxygenase | ammonia |
| Enzyme | Complete LCAT | Lecithin-cholesterol | Cholesterol |
| | deficiency, Fish-eye | acyltransferase (LCAT) | |
| | disease, atherosclerosis, | | |
| | hypercholesterolemia | | |
| Enzyme | Cyanide poisoning | Thiosulfate-cyanide | Cyanide |
| | | sulfurtransferase | |
| Enzyme | Diabetes | Hexokinase, glucokinase | Glucose |
| Enzyme | Factor II Deficiency | FII | enzyme replacement |
| Enzyme | Familial hyperarginemia | Arginase | Arginine |
| Enzyme | Fibrin Stabilizing factor | FXIII | enzyme replacement |
| | Def. | | |
| Enzyme | Glutaric acidemia type I | lysine oxidase | 3-hydroxyglutaric and |
| | | | glutaric acid (C5-DC), |
| | | | lysine |
| Enzyme | Gout | Uricase | Uric Acid |
| Enzyme | Gout - hyperuricemia | Uricase | Uric acid (Urate |
| | | | crystals) |
| Enzyme | Hageman Def. | FXII | enzyme replacement |
| Enzyme | Hemolytic anemia due to | pyrimidine 5′ nucleotidase | pyrimidines |
| | pyrimidine 5′ nucleotidase | | |
| | deficiency | | |
| Enzyme | Hemophilia A | Factor VIII | Thrombin (factor II a) |
| | | | or Factor X |
| Enzyme | Hemophilia B | Factor IX | Factor XIa or Factor X |
| Enzyme | Hemophilia C | FXI | enzyme replacement |
| Enzyme | Hepatocellular carcinoma, | Arginine deiminase | Arginine |
| | melanoma | | |
| Enzyme | Homocystinuria | Cystathionine B synthase | homocysteine |
| Enzyme | hyperammonemia/ornithinemia/ | Ammonia monooxygenase | Ammonia |
| | citrullinemia | | |
| | (ornithine transporter | | |
| | defect) | | |
| Enzyme | Isovaleric acidemia | Leucine metabolizing enzyme | leucine |
| Enzyme | Lead poisoning | d-aminolevulinate | lead |
| | | dehydrogenase | |
| Enzyme | Lesch-Nyhan syndrome | Uricase | Uric acid |
| Enzyme | Maple syrup urine disease | Leucine metabolizing enzyme | Leucine |
| Enzyme | Methylmalonic acidemia | methylmalonyl-CoA mutase | methylmalonate |
| | (vitamin b12 non- | | |
| | responsive) | | |
| Enzyme | Mitochondrial | thymidine phosphorylase | thymidine |
| | neurogastrointestinal | | |
| | encephalomyopathy | | |
| Enzyme | Mitochondrial | Thymidine phosphorylase | Thymidine |
| | neurogastrointestinal | | |
| | encephalomyopathy | | |
| | (MNGIE) | | |
| Enzyme | Owren's disease | FV | enzyme replacement |
| Enzyme | p53-null solid tumor | Serine dehyrdatase or serine | serine |
| | | hydroxymethyl transferase | |
| Enzyme | Pancreatic | Asparaginase | asparagine |
| | adenocarcinoma | | |
| Enzyme | Phenylketonuria | Phenylalanine hydroxylase, | Phenylalanine |
| | | phenylalanine ammonia lyase | |
| Enzyme | Primary hyperoxaluria | Oxalate oxidase | Oxalate |
| Enzyme | Propionic acidemia | Propionate conversion enzyme? | Proprionyl coA |
| Enzyme | Purine nucleoside | Purine nucleoside phosphorylase | Inosine, dGTP |
| | phosphorylase deficiency | | |
| Enzyme | Stuart-Power Def. | FX | enzyme replacement |
| Enzyme | Thrombotic | ADAMTS13 | ultra-large von |
| | Thrombocytopenic | | willebrand factor |
| | Purpura | | (ULVWF) |
| Enzyme | Transferase deficient | galactose dehydrogenase | Galactose-1-phosphate |
| | galactosemia | | |
| | (Galactosemia type 1) | | |
| Enzyme | Tyrosinemia type 1 | tyrosine phenol-lyase | tyrosine |
| Enzyme | von Willebrand disease | vWF | enzyme replacement |
| IC clearance | IgA Nephropathy | Complement receptor 1 | Immune complexes |
| IC clearance | Lupus nephritis | Complement receptor 1 | immune complex |
| IC clearance | Systemic lupus | Complement receptor 1 | immune complex |
| | erythematosus | | |
| Infectious | Anthrax (B. anthracis) | an antibody-like binder to B. anthracis | B. anthracis |
| | infection | surface protein | |
| Infectious | C. botulinum infection | an antibody-like binder to C. botulinum | C. botulinum |
| | | surface protein | |
| Infectious | C. difficile infection | an antibody-like binder to C. difficile | C. difficile |
| | | surface protein | |
| Infectious | Candida infection | an antibody-like binder to | candida |
| | | candida surface protein | |
| Infectious | E. coli infection | an antibody-like binder to E. coli | E. coli |
| | | surface protein | |
| Infectious | Ebola infection | an antibody-like binder to Ebola | Ebola |
| | | surface protein | |
| Infectious | Hepatitis B (HBV) | an antibody-like binder to HBV | HBV |
| | infection | surface protein | |
| Infectious | Hepatitis C (HCV) | an antibody-like binder to HCV | HCV |
| | infection | surface protein | |
| Infectious | Human | an antibody-like binder to HIV | HIV |
| | immunodeficiency virus | envelope proteins or CD4 or | |
| | (HIV) infection | CCR5 or | |
| Infectious | M. tuberculosis infection | an antibody-like binder to M. tuberculosis | M. tuberculosis |
| | | surface protein | |
| Infectious | Malaria (P. falciparum) | an antibody-like binder to P. falciparum | P. falciparum |
| | infection | surface protein | |
| Lipid | Hepatic lipase deficiency, | Hepatic lipase (LIPC) | Lipoprotein, |
| | hypercholesterolemia | | intermediate density |
| | | | (IDL) |
| Lipid | Hyperalphalipoproteinemia 1 | Cholesteryl ester transfer | Lipoprotein, high |
| | | protein(CETP) | density (HDL) |
| Lipid | hypercholesterolemia | an antibody-like binder to low- | LDL |
| | | density lipoprotein (LDL), LDL | |
| | | receptor | |
| Lipid | hypercholesterolemia | an antibody-like binder to high- | HDL |
| | | density lipoprotein (HDL) or | |
| | | HDL receptor | |
| Lipid | lipoprotein lipase | lipoprotein lipase | chilomicrons and very |
| | deficiency | | low density lipoproteins |
| | | | (VLDL) |
| Lipid | Lipoprotein lipase | lipoprotein lipase (LPL) | Lipoprotein, very low |
| | deficiency, disorders of | | density (VLDL) |
| | lipoprotein metabolism | | |
| Lysosomal | Aspartylglucosaminuria | N-Aspartylglucosaminidase | glycoproteins |
| storage | (208400) | | |
| Lysosomal | Cerebrotendinous | Sterol 27-hydroxylase | lipids, cholesterol, and |
| storage | xanthomatosis | | bile acid |
| | (cholestanol lipidosis; | | |
| | 213700) | | |
| Lysosomal | Ceroid lipofuscinosis | Palmitoyl-protein thioesterase-1 | lipopigments |
| storage | Adult form (CLN4, Kufs' | | |
| | disease; 204300) | | |
| Lysosomal | Ceroid lipofuscinosis | Palmitoyl-protein thioesterase-1 | lipopigments |
| storage | Infantile form (CLN1, | | |
| | Santavuori-Haltia disease; | | |
| | 256730) | | |
| Lysosomal | Ceroid lipofuscinosis | Lysosomal transmembrane | lipopigments |
| storage | Juvenile form (CLN3, | CLN3 protein | |
| | Batten disease, Vogt- | | |
| | Spielmeyer disease; | | |
| | 204200) | | |
| Lysosomal | Ceroid lipofuscinosis Late | Lysosomal pepstatin-insensitive | lipopigments |
| storage | infantile form (CLN2, | peptidase | |
| | Jansky-Bielschowsky | | |
| | disease; 204500) | | |
| Lysosomal | Ceroid lipofuscinosis | Transmembrane CLN8 protein | lipopigments |
| storage | Progressive epilepsy with | | |
| | intellectual disability | | |
| | (600143) | | |
| Lysosomal | Ceroid lipofuscinosis | Transmembrane CLN6 protein | lipopigments |
| storage | Variant late infantile form | | |
| | (CLN6; 601780) | | |
| Lysosomal | Ceroid lipofuscinosis | Lysosomal transmembrane | lipopigments |
| storage | Variant late infantile | CLN5 protein | |
| | form, Finnish type | | |
| | (CLN5; 256731) | | |
| Lysosomal | Cholesteryl ester storage | lisosomal acid lipase | lipids and cholesterol |
| storage | disease (CESD) | | |
| Lysosomal | Congenital disorders of | Phosphomannomutase-2 | N-glycosylated protein |
| storage | N-glycosylation CDG Ia | | |
| | (solely neurologic and | | |
| | neurologic-multivisceral | | |
| Lysosomal | Congenital disorders of | α-1,2-Mannosyltransferase | N-glycosylated protein |
| storage | N-glycosylation, type I | | |
| | (pre-Golgi glycosylation | | |
| | defects) | | |
| Lysosomal | Cystinosis | Cystinosin (lysosomal cystine | Cysteine |
| storage | | transporter) | |
| Lysosomal | Fabry's disease (301500) | Trihexosylceramide α- | globotriaosylceramide |
| storage | | galactosidase | |
| Lysosomal | Farber's disease | Ceramidase | lipids |
| storage | (lipogranulomatosis; | | |
| | 228000) | | |
| Lysosomal | Fucosidosis (230000) | α-L-Fucosidase | fucose and complex |
| storage | | | sugars |
| Lysosomal | Galactosialidosis | Protective protein/cathepsin A | lysosomal content |
| storage | (Goldberg's syndrome, | (PPCA) | |
| | combined neuraminidase | | |
| | and β-galactosidase | | |
| | deficiency; 256540) | | |
| Lysosomal | Gaucher's disease | Glucosylceramide β-glucosidase | sphingolipids |
| storage | | | |
| Lysosomal | Glutamyl ribose-5- | ADP-ribose protein hydrolase | glutamyl ribose 5- |
| storage | phosphate storage disease | | phosphate |
| | (305920) | | |
| Lysosomal | Glycogen storage disease | alpha glucosidase | glycogen |
| storage | type 2 (Pompe's disease) | | |
| Lysosomal | GM1 gangliosidosis, | Ganglioside β-galactosidase | acidic lipid material, |
| storage | generalized | | gangliosides |
| Lysosomal | GM2 activator protein | GM2 activator protein | gangliosides |
| storage | deficiency (Tay-Sachs | | |
| | disease AB variant, | | |
| | GM2A; 272750) | | |
| Lysosomal | GM2 gangliosidosis | Ganglioside β-galactosidase | gangliosides |
| storage | | | |
| Lysosomal | Infantile sialic acid | Na phosphate cotransporter, | sialic acid |
| storage | storage disorder (269920) | sialin | |
| Lysosomal | Krabbe's disease (245200) | Galactosylceramide β- | sphingolipids |
| storage | | galactosidase | |
| Lysosomal | Lysosomal acid lipase | Lysosomal acid lipase | cholesteryl |
| storage | deficiency (278000) | | esters and triglycerides |
| Lysosomal | Metachromatic | Arylsulfatase A | sulfatides |
| storage | leukodystrophy (250100) | | |
| Lysosomal | Mucolipidosis ML II (I- | N-Acetylglucosaminyl-1- | N-linked glycoproteins |
| storage | cell disease; 252500) | phosphotransfeerase catalytic | |
| | | subunit | |
| Lysosomal | Mucolipidosis ML III | N-acetylglucosaminyl-1- | N-linked glycoproteins |
| storage | (pseudo-Hurler's | phosphotransfeerase | |
| | polydystrophy) | | |
| Lysosomal | Mucolipidosis ML III | Catalytic subunit | N-linked glycoproteins |
| storage | (pseudo-Hurler's | | |
| | polydystrophy) Type III- | | |
| | A (252600) | | |
| Lysosomal | Mucolipidosis ML III | Substrate-recognition subunit | N-linked glycoproteins |
| storage | (pseudo-Hurler's | | |
| | polydystrophy) Type III- | | |
| | C (252605) | | |
| Lysosomal | Mucopolysaccharidosis | α-1-Iduronidase | glycosaminoglycans |
| storage | MPS I H/S (Hurler-Scheie | | |
| | syndrome; 607015) | | |
| Lysosomal | Mucopolysaccharidosis | α-1-Iduronidase | glycosaminoglycans |
| storage | MPS I-H (Hurler's | | |
| | syndrome; 607014) | | |
| Lysosomal | Mucopolysaccharidosis | Iduronate sulfate sulfatase | glycosaminoglycans |
| storage | MPS II (Hunter's | | |
| | syndrome; 309900) | | |
| Lysosomal | Mucopolysaccharidosis | Heparan-S-sulfate sulfamidase | glycosaminoglycans |
| storage | MPS III (Sanfilippo's | | |
| | syndrome) Type III-A | | |
| | (252900) | | |
| Lysosomal | Mucopolysaccharidosis | N-acetyl-D-glucosaminidase | glycosaminoglycans |
| storage | MPS III (Sanfilippo's | | |
| | syndrome) Type III-B | | |
| | (252920) | | |
| Lysosomal | Mucopolysaccharidosis | Acetyl-CoA-glucosaminide N- | glycosaminoglycans |
| storage | MPS III (Sanfilippo's | acetyltransferase | |
| | syndrome) Type III-C | | |
| | (252930) | | |
| Lysosomal | Mucopolysaccharidosis | N-acetyl-glucosaminine-6- | glycosaminoglycans |
| storage | MPS III (Sanfilippo's | sulfate sulfatase | |
| | syndrome) Type III-D | | |
| | (252940) | | |
| Lysosomal | Mucopolysaccharidosis | α-1-Iduronidase | glycosaminoglycans |
| storage | MPS I-S (Scheie's | | |
| | syndrome; 607016) | | |
| Lysosomal | Mucopolysaccharidosis | Galactosamine-6-sulfate | glycosaminoglycans |
| storage | MPS IV (Morquio's | sulfatase | |
| | syndrome) Type IV-A | | |
| | (253000) | | |
| Lysosomal | Mucopolysaccharidosis | β-Galactosidase | glycosaminoglycans |
| storage | MPS IV (Morquio's | | |
| | syndrome) Type IV-B | | |
| | (253010) | | |
| Lysosomal | Mucopolysaccharidosis | Hyaluronidase deficiency | glycosaminoglycans |
| storage | MPS IX (hyaluronidase | | |
| | deficiency; 601492) | | |
| Lysosomal | Mucopolysaccharidosis | N-Acetyl galactosamine α-4- | glycosaminoglycans |
| storage | MPS VI (Maroteaux- | sulfate sulfatase (arylsulfatase B) | |
| | Lamy syndrome; 253200) | | |
| Lysosomal | Mucopolysaccharidosis | β-Glucuronidase | glycosaminoglycans |
| storage | MPS VII (Sly's syndrome; | | |
| | 253220) | | |
| Lysosomal | Mucosulfatidosis | Sulfatase-modifying factor-1 | sulfatides |
| storage | (multiple sulfatase | | |
| | deficiency; 272200) | | |
| Lysosomal | Niemann-Pick disease | Sphingomyelinase | sphingomyelin |
| storage | type A | | |
| Lysosomal | Niemann-Pick disease | Sphingomyelinase | sphingomyelin |
| storage | type B | | |
| Lysosomal | Niemann-Pick disease | NPC1 protein | sphingomyelin |
| storage | Type C1/Type D | | |
| | ((257220) | | |
| Lysosomal | Niemann-Pick disease | Epididymal secretory protein 1 | sphingomyelin |
| storage | Type C2 (607625) | (HE1; NPC2 protein) | |
| Lysosomal | Prosaposin deficiency | Prosaposin | sphingolipids |
| storage | (176801) | | |
| Lysosomal | Pycnodysostosis (265800) | Cathepsin K | kinins |
| storage | | | |
| Lysosomal | Sandhoff's disease; | β-Hexosaminidase B | gangliosides |
| storage | 268800 | | |
| Lysosomal | Saposin B deficiency | Saposin B | sphingolipids |
| storage | (sulfatide activator | | |
| | deficiency) | | |
| Lysosomal | Saposin C deficiency | Saposin C | sphingolipids |
| storage | (Gaucher's activator | | |
| | deficiency) | | |
| Lysosomal | Schindler's disease Type I | N-Acetyl-galactosaminidase | glycoproteins |
| storage | (infantile severe form; | | |
| | 609241) | | |
| Lysosomal | Schindler's disease Type | N-Acetyl-galactosaminidase | glycoproteins |
| storage | II (Kanzaki disease, adult- | | |
| | onset form; 609242) | | |
| Lysosomal | Schindler's disease Type | N-Acetyl-galactosaminidase | glycoproteins |
| storage | III (intermediate form; | | |
| | 609241) | | |
| Lysosomal | Sialidosis (256550) | Neuraminidase 1 (sialidase) | mucopolysaccharides |
| storage | | | and mucolipids |
| Lysosomal | Sialuria Finnish type | Na phosphate cotransporter, | sialic acid |
| storage | (Salla disease; 604369) | sialin | |
| Lysosomal | Sialuria French type | UDP-N-acetylglucosamine-2- | sialic acid |
| storage | (269921) | epimerase/N-acetylmannosamine | |
| | | kinase, sialin | |
| Lysosomal | Sphingolipidosis Type I | Ganglioside β-galactosidase | sphingolipids |
| storage | (230500) | | |
| Lysosomal | Sphingolipidosis Type II | Ganglioside β-galactosidase | sphingolipids |
| storage | (juvenile type; 230600) | | |
| Lysosomal | Sphingolipidosis Type III | Ganglioside β-galactosidase | sphingolipids |
| storage | (adult type; 230650) | | |
| Lysosomal | Tay-Sachs disease; | β-Hexosaminidase A | gangliosides |
| storage | 272800 | | |
| Lysosomal | Winchester syndrome | Metalloproteinase-2 | mucopolysaccharides |
| storage | (277950) | | |
| Lysosomal | Wolman's disease | lysosomal acid lipase | lipids and cholesterol |
| storage | | | |
| Lysosomal | α-Mannosidosis (248500), | α-D-Mannosidase | carbohydrates and |
| storage | type I (severe) or II (mild) | | glycoproteins |
| Lysosomal | β-Mannosidosis (248510) | β-D-Mannosidase | carbohydrates and |
| storage | | | glycoproteins |
| Toxic | alpha hemolysin | an antibody-like binder to alpha | alpha hemolysin |
| Molecule | poisoning | hemolysin | |
| Toxic | antrax toxin poisoning | an antibody-like binder to | anthrax toxin |
| Molecule | | anthrax toxin | |
| Toxic | bacterial toxin-induced | an antibody-like binder to | bacterial toxin |
| Molecule | shock | bacterial toxin | |
| Toxic | botulinum toxin poisoning | an antibody-like binder to | botulinum toxin |
| Molecule | | botulinum toxin | |
| Toxic | Hemochromatosis (iron | iron chelator | molecular iron |
| Molecule | poisoning) | | |
| Toxic | Methanol poisoning | Methanol dehdrogenase | Methanol |
| Molecule | | | |
| Toxic | Nerve gas poisoning | Butyryl cholinesterase | Sarin |
| Molecule | | | |
| Toxic | Prion disease caused by | an antibody-like binder to prion | Prion protein PRP |
| Molecule | PRP | protein PRP | |
| Toxic | Prion disease caused by | an antibody-like binder to prion | Prion protein PRPc |
| Molecule | PRPc | protein PRPc | |
| Toxic | Prion disease caused by | an antibody-like binder to prion | Prion protein PRPsc |
| Molecule | PRPsc | protein PRPsc | |
| Toxic | Prion disease caused by | an antibody-like binder to prion | Prion protein PRPres |
| Molecule | PRPres | protein PRPres | |
| Toxic | Sepsis or cytokine storm | an antibody-like binder to | cytokines |
| Molecule | | cytokines or Duffy antigen | |
| | | receptor of chemokines (DARC) | |
| Toxic | spider venom poisoning | an antibody-like binder to spider | spider venom |
| Molecule | | venom | |
| Toxic | Wilson disease | copper chelator | molecular copper |
| Molecule | | | |

TABLE 5
Electroporation Conditions (Day 8-9)

| | | Pulse | | | Cell |
|---|---|---|---|---|---|
| Sample | Pulse Voltage | width | Pulse number | % GFP | viability |
| 1 | No | | | 0.21 | 97.39 |
| | electroporation | | | | |
| 2 | 1400 | 20 | 1 | 86.9 | 92.6 |
| 3 | 1500 | 20 | 1 | 79.5 | 85.7 |
| 4 | 1600 | 20 | 1 | 68.2 | 78.5 |
| 5 | 1700 | 20 | 1 | 41.4 | 52.3 |
| 6 | 1100 | 30 | 1 | 79 | 96 |
| 7 | 1200 | 30 | 1 | 83.6 | 91.9 |
| 8 | 1300 | 30 | 1 | 77.6 | 86.6 |
| 9 | 1400 | 30 | 1 | 30.9 | 42.7 |
| 10 | 1000 | 40 | 1 | 65.3 | 92.4 |
| 11 | 1100 | 40 | 1 | 69.3 | 86.9 |
| 12 | 1200 | 40 | 1 | 65.8 | 79.9 |
| 13 | 1100 | 20 | 2 | 81.3 | 92.8 |
| 14 | 1200 | 20 | 2 | 82.1 | 91.2 |
| 15 | 1300 | 20 | 2 | 78.2 | 86.3 |
| 16 | 1400 | 20 | 2 | 79.3 | 88.1 |
| 17 | 850 | 30 | 2 | 32.4 | 95.9 |
| 18 | 950 | 30 | 2 | 59.5 | 93.8 |
| 19 | 1050 | 30 | 2 | 72 | 90.8 |
| 20 | 1150 | 30 | 2 | 74.8 | 84.8 |
| 21 | 1300 | 10 | 3 | 88.3 | 94.2 |
| 22 | 1400 | 10 | 3 | 88.7 | 93.3 |
| 23 | 1500 | 10 | 3 | 86.5 | 90.3 |
| 24 | 1600 | 10 | 3 | 83.3 | 87.7 |

TABLE 6
Electroporation Conditions (Day 12-13)

| | | Pulse | | | Cell |
|---|---|---|---|---|---|
| Sample | Pulse Voltage | width | Pulse number | % GFP | viability |
| 1 | No | | | 0.58 | 96.7 |
| | electroporation | | | | |
| 2 | 1400 | 20 | 1 | 42.5 | 94.9 |
| 3 | 1500 | 20 | 1 | 54.8 | 91.8 |
| 4 | 1600 | 20 | 1 | 56.9 | 91.6 |
| 5 | 1700 | 20 | 1 | 61.5 | 88.7 |
| 6 | 1100 | 30 | 1 | 13.5 | 95.6 |
| 7 | 1200 | 30 | 1 | 29 | 95.5 |
| 8 | 1300 | 30 | 1 | 43.8 | 94.3 |
| 9 | 1400 | 30 | 1 | 44.5 | 92.9 |
| 10 | 1000 | 40 | 1 | 6.5 | 95.3 |
| 11 | 1100 | 40 | 1 | 21.7 | 94.8 |
| 12 | 1200 | 40 | 1 | 33.2 | 92.3 |
| 13 | 1100 | 20 | 2 | 18 | 95.8 |
| 14 | 1200 | 20 | 2 | 29.3 | 95.2 |
| 15 | 1300 | 20 | 2 | 42 | 94.5 |
| 16 | 1400 | 20 | 2 | 51.5 | 91.8 |
| 17 | 850 | 30 | 2 | 2.7 | 95.9 |
| 18 | 950 | 30 | 2 | 7.3 | 95.3 |
| 19 | 1050 | 30 | 2 | 13.5 | 94.5 |
| 20 | 1150 | 30 | 2 | 20.7 | 94.7 |
| 21 | 1300 | 10 | 3 | 27.3 | 95.9 |
| 22 | 1400 | 10 | 3 | 38.8 | 95.3 |
| 23 | 1500 | 10 | 3 | 55 | 94.1 |
| 24 | 1600 | 10 | 3 | 62.6 | 93.3 |

TABLE 7
Electroporation Conditions (Day 14-16)

| | | Pulse | Pulse | | Cell |
|---|---|---|---|---|---|
| Sample | Pulse Voltage | width | number | % GFP | viability |
| 0 | No electroporation | | | 1.1 | 5.2 |
| 1 | 1700 | 20 | 1 | 44.7 | 7.7 |
| 2 | 1700 | 20 | 2 | 44.1 | 15.5 |
| 3 | 1700 | 20 | 3 | 42.7 | 25 |
| 4 | 1600 | 10 | 3 | 37.6 | 7.6 |
| 5 | 1600 | 10 | 6 | 34.9 | 19.1 |
| 6 | 1600 | 10 | 8 | 20.1 | 47.8 |
| 7 | 1600 | 20 | 1 | 36.7 | 5.7 |
| 8 | 1600 | 20 | 2 | 37.2 | 14.6 |
| 9 | 1600 | 20 | 3 | 40.2 | 13 |
| 10 | 1700 | 10 | 1 | 21.7 | 4.9 |
| 11 | 1700 | 10 | 2 | 43 | 9.7 |
| 12 | 1700 | 10 | 3 | 24.9 | 33.9 |

TABLE 8
GFP fluorescence of electroporated Day 4 cells.

| | | % GFP+ | | |
|---|---|---|---|---|
| | % P1 | cells | MFI | % AAD- |
| Non-electroporated control | 87.3 | 0.85 | 2,678 | 98.6 |
| Electroporated, condition A, | 79 | 91.6 | 121,279 | 98.6 |
| trial 1 | | | | |
| Electroporated, condition A, | 80.8 | 90.6 | 105,741 | 98.5 |
| trial 2 | | | | |
| Electroporated, condition B, | 83.5 | 58.8 | 25,482 | 98.4 |
| trial 1 | | | | |
| Electroporated, condition B, | 85.9 | 19.6 | 10,709 | 98.7 |
| trial 2 | | | | |
| Electroporated, condition C, | 87 | 35 | 17,086 | 98.7 |
| trial 1 | | | | |
| Electroporated, condition C, | 86.3 | 13.1 | 8,114 | 98.8 |
| trial 2 | | | | |

TABLE 9
GFP fluorescence of Day 4 cells electroporated with chemically
modified RNA

| | | % GFP+ | | |
|---|---|---|---|---|
| | % P1 | cells | MFI | % AAD- |
| Non-electroporated control | 87.3 | 0.85 | 2,678 | 98.6 |
| Electroporated, condition A, | 87.2 | 96.6 | 75,393 | 98.0 |
| trial 1 | | | | |
| Electroporated, condition A, | 87.4 | 96.3 | 75,853 | 98.5 |
| trial 2 | | | | |
| Electroporated, condition B, | 88.4 | 60.8 | 23,097 | 98.9 |
| trial 1 | | | | |
| Electroporated, condition B, | 87.6 | 57.8 | 21,759 | 98.7 |
| trial 2 | | | | |
| Electroporated, condition C, | 88.7 | 61 | 24,857 | 98.8 |
| trial 1 | | | | |
| Electroporated, condition C, | 88.4 | 50.9 | 20,358 | 98.5 |
| trial 2 | | | | |

TABLE 10
GFP fluorescence of Day 12 cells electroporated with chemically
modified RNA

| | % P1 | % GFP+ cells | MFI | % AAD- |
|---|---|---|---|---|
| Non-electroporated | 92.2 | 0.86 | 3,754 | 95 |
| control | | | | |
| Electroporated, trial 1 | 93.7 | 55.2 | 22,748 | 98 |
| Electroporated, trial 2 | 90.7 | 90 | 107,091 | 94 |

TABLE 11
Evaluation of cell viability and proliferation ability by trypan blue staining
after electroporation

| | Day 8 | Day 9 | Day 9 | Day 9 |
|---|---|---|---|---|
| | Total | Total | Live | Cell |
| | cells (M) | Cells (M) | Cells (M) | viability |
| Electroporated without | 0.21 | 0.441 | 0.441 | 100 |
| exogenous nucleic acid | | | | |
| Electroporated with | 0.2 | 0.376 | 0.37 | 99 |
| unmodified GFP mRNA, | | | | |
| 1 ug | | | | |
| Electroporated with | 0.2 | 0.354 | 0.332 | 94 |
| unmodified GFP mRNA, | | | | |
| 2 ug | | | | |
| Electroporated with | 0.2 | 0.414 | 0.381 | 92 |
| modified GFP mRNA, 1 ug | | | | |

TABLE 12
human noncoding RNAs

A1BG-AS1 A2M-AS1 A2ML1-AS1 AADACL2-AS1 AATK-AS1 ABCA9-AS1 ABCC5-AS1 ABHD11-AS1
ABHD14A-ACY1 ABHD15-AS1 ACAP2-IT1 ACTA2-AS1 ACTN1-AS1 ACVR2B-AS1 LOC100130964
ADAMTS19-AS1 ADAMTS9-AS1 ADAMTS9-AS2 ADAMTSL4-AS1 ADARB2-AS1 ADD3-AS1 ADGRA1-
AS1 ADGRL3-AS1 ADIPOQ-AS1 ADIRF-AS1 ADNP-AS1 ADORA2A-AS1 ADPGK-AS1 LOC104968398
AFAP1-AS1 AFF2-IT1 AGAP1-IT1 AGAP2-AS1 AGBL1-AS1 AGBL4-IT1 AGBL5-AS1 AGPAT4-IT1 AKT3-
IT1 ALDH1L1-AS1 ALDH1L1-AS2 ALG9-IT1 ALKBH3-AS1 ALMS1-IT1 ALOX12-AS1 APTR AMMECR1-
IT1 ANKRD10-IT1 ANKRD33B-AS1 ANKRD34C-AS1 ANKRD44-IT1 ANO1-AS1 ANO1-AS2 ANP32A-IT1
LOC280665 AIRN UCH1LAS AOAH-IT1 AP4B1-AS1 APCDD1L-AS1 APOA1-AS APOBEC3B-AS1 APOC4-
APOC2 AATBC ABALON AQP4-AS1 ARAP1-AS1 ARAP1-AS2 ARHGAP19-SLIT1 ARHGAP22-IT1
ARHGAP26-AS1 ARHGAP26-IT1 ARHGAP31-AS1 ARHGAP5-AS1 ARHGEF19-AS1 ARHGEF26-AS1
ARHGEF3-AS1 ARHGEF38-IT1 ARHGEF7-AS1 ARHGEF7-AS2 ARHGEF7-IT1 ARHGEF9-IT1 ARID4B-IT1
ARL2-SNX15 ARMC2-AS1 ARNTL2-AS1 ARPP21-AS1 ARRDC1-AS1 ARRDC3-AS1 ARSD-AS1 ASAP1-IT1
ASAP1-IT2 ASB16-AS1 ASH1L-AS1 ASMTL-AS1 ASTN2-AS1 ATE1-AS1 ATG10-AS1 ATG10-IT1 ATP11A-
AS1 ATP13A4-AS1 ATP13A5-AS1 ATP1A1-AS1 ATP1B3-AS1 ATP2A1-AS1 ATP2B2-IT1 ATP2B2-IT2
ATP6V0E2-AS1 ATP6V1B1-AS1 ATP6V1G2-DDX39B ATXN8OS AZIN1-AS1 LOC100506431 B3GALT5-AS1
B4GALT1-AS1 B4GALT4-AS1 BAALC-AS1 BAALC-AS2 BACE1-AS BACE2-IT1 BACH1-IT2 BACH1-IT3
BAIAP2-AS1 BARX1-AS1 BBOX1-AS1 BCDIN3D-AS1 BLACE BDNF-AS BEAN1-AS1 BGLT3 BHLHE40-
AS1 BIN3-IT1 BIRC6-AS1 BIRC6-AS2 BLACAT1 BPESC1 BLOC1S1-RDH5 BLOC1S5-TXNDC5 BMP7-AS1
BMPR1B-AS1 BOK-AS1 BOLA3-AS1 BANCR BCYRN1 BRE-AS1 BCAR4 BREA2 BRWD1-AS1 BRWD1-IT2
BSN-AS2 BISPR BTBD9-AS1 BVES-AS1 BZRAP1-AS1 C10orf32-ASMT C10orf71-AS1 C15orf59-AS1
C1QTNF1-AS1 C1QTNF3-AMACR C1QTNF9-AS1 C1RL-AS1 C2-AS1 C20orf166-AS1 C21orf62-AS1
C21orf91-OT1 C3orf67-AS1 C5orf66-AS1 C5orf66-AS2 C8orf34-AS1 C8orf37-AS1 C9orf135-AS1 C9orf173-
AS1 C9orf41-AS1 CA3-AS1 CACNA1C-AS1 CACNA1C-AS2 CACNA1C-AS4 CACNA1C-IT1 CACNA1C-IT2
CACNA1C-IT3 CACNA1G-AS1 CACNA2D3-AS1 CACTIN-AS1 CADM2-AS1 CADM2-AS2 CADM3-AS1
CALML3-AS1 CAMTA1-IT1 CASC11 CASC15 CASC16 CASC17 CASC18 CASC19 CASC2 CASC20 CASC21
CASC23 CASC6 CASC8 CASC9 CAPN10-AS1 CARD8-AS1 CARS-AS1 CASK-AS1 CECR3 CECR7 CATIP-
AS1 CATIP-AS2 CATR1 CBR3-AS1 CCDC13-AS1 CCDC144NL-AS1 CCDC148-AS1 CCDC183-AS1 CCDC26
CCDC37-AS1 CCDC39-AS1 CCL15-CCL14 CCND2-AS1 CCNT2-AS1 CD27-AS1 CD81-AS1 CDC37L1-AS1
CDC42-IT1 CDH23-AS1 CDIPT-AS1 CDKN1A-AS1 CDKN2A-AS1 CDKN2B-AS1 CDR1-AS CEBPA-AS1
CEBPB-AS1 CEBPZOS CECR5-AS1 CELF2-AS1 CELF2-AS2 CELSR3-AS1 CEP83-AS1 CERS3-AS1 CERS6-
AS1 CCHE1 CFAP44-AS1 CFAP58-AS1 CFLAR-AS1 CHKB-AS1 CHKB-CPT1B CHL1-AS1 CHODL-AS1
CISTR CHRM3-AS1 CHRM3-AS2 Clorf145 C1orf220 C11orf39 C11orf72 C14orf144 C18orf15 C3orf49 C5orf17
C5orf56 C6orf7 C7orf13 C8orf49 CIRBP-AS1 CKMT2-AS1 CLDN10-AS1 CLIP1-AS1 CLSTN2-AS1 CLUHP3
CLYBL-AS1 CLYBL-AS2 CDRT7 CDRT8 CNOT10-AS1 CNTFR-AS1 CNTN4-AS1 CNTN4-AS2 COL18A1-
AS1 COL18A1-AS2 COL4A2-AS1 COL5A1-AS1 LOC387720 CAHM CCAT1 CCAT2 CRNDE COPG2IT1
COX10-AS1 CPB2-AS1 CPEB1-AS1 CPEB2-AS1 CPS1-IT1 CRHR1-IT1 CRTC3-AS1 CRYM-AS1 CSE1L-AS1
CSMD2-AS1 CSNK1G2-AS1 CSTF3-AS1 CTBP1-AS CTBP1-AS2 CYP17A1-AS1 CYP1B1-AS1 CYP4A22-AS1
CYP51A1-AS1 D21S2088E DBET DAB1-AS1 DACT3-AS1 DAOA-AS1 DAPK1-IT1 DARS-AS1 DBH-AS1
DCTN1-AS1 DCUN1D2-AS DDC-AS1 DDX11-AS1 DDX26B-AS1 LOC642846 DLEU1 DLEU2 DENND5B-
AS1 DEPDC1-AS1 DGUOK-AS1 DHRS4-AS1 DIAPH2-AS1 DIAPH3-AS1 DIAPH3-AS2 DICER1-AS1 DANCR
DGCR10 DGCR11 DGCR7 DGCR9 DIO2-AS1 DIO3OS DIP2A-IT1 DISC1FP1 DISC1-IT1 DIRC3 DISC2 PKI55
DLEU1-AS1 DLEU7-AS1 DLG1-AS1 DLG3-AS1 DLG5-AS1 DLGAP1-AS1 DLGAP1-AS2 DLGAP1-AS3
DLGAP1-AS4 DLGAP1-AS5 DLGAP2-AS1 DLGAP4-AS1 DLX2-AS1 DLX6-AS1 DLX6-AS2 DMD-AS3
DNAH17-AS1 DNAJB5-AS1 DNAJB8-AS1 DNAJC27-AS1 DNAJC3-AS1 DNAJC9-AS1 DNM3-IT1 DNM3OS
DNMBP-AS1 DALIR DOCK4-AS1 DOCK9-AS1 DOCK9-AS2 DSCR10 DSCR8 DSCR9 DRAIC DPH6-AS1
DPP10-AS1 DPP10-AS3 DPYD-AS1 DPYD-AS2 DPYD-IT1 DSCAS DSCAM-AS1 DSCAM-IT1 DSCR4-IT1
DSG1-AS1 DSG2-AS1 DNAH10OS DYX1C1-CCPG1 ERICD E2F3-IT1 EAF1-AS1 EDNRB-AS1 EDRF1-AS1
EEF1E1-BLOC1S5 EFCAB14-AS1 EFCAB6-AS1 EFCAB10 EGFLAM-AS1 EGFLAM-AS2 EGFLAM-AS3
EGFLAM-AS4 EGFR-AS1 ELDR EHD4-AS1 EHHADH-AS1 EHMT1-IT1 EIF1AX-AS1 EIF1B-AS1 EIF2B5-
AS1 EIF2B5-IT1 EIF3J-AS1 ELFN1-AS1 ELMO1-AS1 ELOVL2-AS1 ESRG EMC3-AS1 EML2-AS1 EMX2OS
LOC105376387 ERVK13-1 ENO1-AS1 ENOX1-AS1 ENOX1-AS2 ENTPD1-AS1 ENTPD3-AS1 EGOT EP300-
AS1 EPB41L4A-AS1 EPB41L4A-AS2 EPHAl-AS1 EPHA5-AS1 EPN2-AS1 EPN2-IT1 ERC2-IT1 ERI3-IT1
ERICH1-AS1 ERICH3-AS1 ERICH6-AS1 ETV5-AS1 EVX1-AS EWSAT1 EXOC3-AS 1 EXTL3-AS1 EZR-AS1
F10-AS1 F11-AS1 FAM13A-AS1 FAM155A-IT1 FAM167A-AS1 FAM170B-AS1 FAM181A-AS1 FAM212B-
AS1 FAM222A-AS1 FAM24B-CUZD1 FAM53B-AS1 FAM83A-AS1 FAM83C-AS1 FAM83H-AS1 FAM106A
FAM106B FAM106CP FAM138A FAM138B FAM138C FAM138D FAM138E FAM138F FAM157C FAM182A
FAM182B FAM183CP FAM197Y4 FAM197Y5 FAM197Y7 FAM201A FAM215A FAM223A FAM223B
FAM224A FAM224B FAM225A FAM225B FAM226A FAM226B FAM230B FAM230C FAM231D FAM27B
FAM27C FAM27D1 FAM27E2 FAM27E3 FAM27L FAM41AY1 FAM41AY2 FAM41C FAM66A FAM66B
FAM66C FAM66D FAM66E FAM74A1 FAM74A3 FAM74A4 FAM74A6 FAM74A7 FAM85A FAM87A
FAM87B FAM95A FAM95B1 FAM95C FAM99A FAM99B FANK1-AS1 FARP1-AS1 FAS-AS1 FAR2P1
FAR2P2 FAR2P3 FBXL19-AS1 FBXO22-AS1 FBXO3-AS1 FBXO36-IT1 FER1L6-AS1 FER1L6-AS2 FEZF1-
AS1 FGD5-AS1 FGF10-AS1 FGF12-AS1 FGF12-AS2 FGF12-AS3 FGF13-AS1 FGF14-AS1 FGF14-AS2 FGF14-
IT1 FIRRE FKBP1A-SDCBP2 FKSG29 FLG-AS1 FLJ16171 FLJ26850 FLJ33360 FLJ35934 FLJ43879 FLJ45079
FLNB-AS1 FLVCR1-AS1 FMR1-AS1 FNDC1-IT1 FOCAD-AS1 FALEC FTCDNL1 FOXCUT FOXC2-AS1
FOXD2-AS1 FOXD3-AS1 FENDRR FOXG1-AS1 FOXN3-AS1 FOXN3-AS2 FOXP1-AS1 FOXP4-AS1 FREM2-
AS1 FRMD6-AS1 FRMD6-AS2 FRMPD3-AS1 FRMPD4-AS1 FRY-AS1 FSIP2-AS1 FTCD-AS1 FTO-IT1 FTX
FUT8-AS1 FZD10-AS1 GABPB1-AS1 GABRG3-AS1 GAS5-AS1 GAS6-AS1 GAS6-AS2 GAS8-AS1 GAPLINC
GACAT1 GACAT2 GACAT3 GCRG224 GHET1 GATA2-AS1 GATA3-AS1 GATA6-AS1 GCSAML-AS1
GAEC1 GFOD1-AS1 GHRLOS GJA9-MYCBP GK-AS1 GLIDR GLIS2-AS1 GLIS3-AS1 GLYCTK-AS1 GMDS-
AS1 GNA14-AS1 GNAS-AS1 GNG12-AS1 GPC5-AS1 GPC5-AS2 GPC5-IT1 GPC6-AS1 GPC6-AS2 GPR1-AS
GPR158-AS1 GRID1-AS1 GRIK1-AS1 GRIK1-AS2 GRK5-IT1 GRM5-AS1 GRM7-AS1 GRM7-AS2 GRM7-AS3
GASS GDF5OS GRTP1-AS1 GSN-AS1 GTF3C2-AS1 GTSE1-AS1 GYG2-AS1 H19 H1FX-AS1 HAGLROS
HAND2-AS1 HAO2-IT1 HAS2-AS1 HCFC1-AS1 LOC100131635 LOC646268 LOC728040 LOC100130298
LOC729970 LOC727925 HDAC11-AS1 HECTD2-AS1 HPYR1 HELLPAR HCCAT5 HEIH HULC HEXA-AS1
HEXDC-IT1 HHATL-AS1 HHIP-AS1 HID1-AS1 HIF1A-AS1 HIF1A-AS2 HAR1A HAR1B HIPK1-AS1 HCG11
HCG14 HCG17 HCG18 HCG20 HCG21 HCG22 HCG23 HCG24 HCG25 HCG26 HCG27 HCG4 HCG4B HCG8
HCG9 HCP5 HLA-F-AS1 HLCS-IT1 HLTF-AS1 HLX-AS1 HM13-AS1 HMGN3-AS1 HMMR-AS1 HNF1A-AS1
HNF4A-AS1 HNRNPU-AS1 HNRNPUL2-BSCL2 HORMAD2-AS1 HOTAIR HOXA-AS2 HOXA-AS3 HOTTIP
HOTAIRM1 HOXA10-AS HOXA10-HOXA9 HOXA11-AS HOXB-AS1 HOXB-AS2 HOXB-AS3 HOXB-AS4
HOXC-AS1 HOXC-AS2 HOXC-AS3 HOXC13-AS HAGLR HOXD-AS2 HPN-AS1 HS1BP3-IT1 HS65T2-AS1
HSPB2-C11orf52 HTR2A-AS1 HTR5A-AS1 HTT-AS HPVC1 HYMAI HYI-AS1 IBAS7-AS1 ID2-AS1 IDH1-
AS1 IDI2-AS1 IFNG-AS1 IFT74-AS1 IGBP1-AS1 TRAIN IGF2-AS IGF2BP2-AS1 IGFBP7-AS1 IGSF11-AS1
IL10RB-AS1 IL12A-AS1 IL21-AS1 IL21R-AS1 ILF3-AS1 IPW INE1 INE2 INHBA-AS1 INMT-FAM188B
INO80B-WBP1 INTS6-AS1 IPO11-LRRC70 IPO9-AS1 IQCF5-AS1 IQCH-AS1 IQCJ-SCHIP1-AS1 ISM1-AS1
ISPD-AS1 ISX-AS1 ITCH-IT1 ITFG1-AS1 ITGA9-AS1 ITGB2-AS1 ITGB5-AS1 ITIH4-AS1 ITPK1-AS1 ITPKB-
AS1 ITPKB-IT1 ITPR1-AS1 JADRR JAKMIP2-AS1 JARID2-AS1 JAZF1-AS1 JHDM1D-AS1 JMJD1C-AS1 JPX
JRKL-AS1 KANSL1-AS1 KBTBD11-OT1 KCNAB1-AS1 KCNAB1-AS2 KCNC4-AS1 KCND3-AS1 KCND3-
IT1 KCNH1-IT1 KCNIP2-AS1 KCNIP4-IT1 KCNJ2-AS1 KCNJ6-AS1 KCNMA1-AS1 KCNMA1-AS2
KCNMA1-AS3 KCNMB2-AS1 KCNQ1-AS1 KCNQ1DN KCNQ10T1 KCNQ5-AS1 KCNQ5-IT1 KCTD21-AS1
KDM4A-AS1 KANTR KDM5C-IT1 KC6 KIAA0087 KIAA0125 KIAA1656 KIAA1875 KIF25-AS1 KIF9-AS1
KIRREL3-AS1 KIRREL3-AS2 KIRREL3-AS3 KIZ-AS1 KLF3-AS1 KLF7-IT1 KLHL30-AS1 KLHL6-AS1
KLHL7-AS1 KMT2E-AS1 KRBOX1-AS1 KRT73-AS1 KRTAP5-AS1 KTN1-AS1 L3MBTL4-AS1 LACTB2-AS1
LAMA5-AS1 LAMP5-AS1 LAMTOR5-AS1 LANCL1-AS1 LARGE-AS1 LARGE-IT1 LARS2-AS1 LATS2-AS1
LBX1-AS1 LBX2-AS1 LCMT1-AS1 LCMT1-AS2 LDLRAD4-AS1 LEF1-AS1 LEMD1-AS1 LENG8-AS1
LRRC37A5P LUNAR1 LGALS8-AS1 LHFPL3-AS1 LHFPL3-AS2 LHX4-AS1 LHX5-AS1 LIFR-AS1 LIMD1-
AS1 LIMS3-LOC440895 LINGO1-AS1 LINGO1-AS2 LIPE-AS1 LLPH-AS1 LMCD1-AS1 LMF1-AS1 LMLN-
AS1 LMO7-AS1 LMO7DN-IT1 LNX1-AS1 LNX1-AS2 LOC100630923 LOC100499484-C9ORF174 LINC01000
LINC01001 LINC01002 LINC01003 LINC01004 LINC01005 LINC01006 LINC01007 LINC01010 LINC01011
LINC01012 LINC01013 LINC01014 LINC01015 LINC01016 LINC01017 LINC01018 LINC01019 LINC00102
LINC01020 LINC01021 LINC01023 LINC01024 LINC01028 LINC01029 LINC01030 LINC01031 LINC01032
LINC01033 LINC01036 LINC01038 LINC01039 LINC01040 LINC01043 LINC01044 LINC01046 LINC01047
LINC01048 LINC01049 LINC01050 LINC01053 LINC01054 LINC01055 LINC01056 LINC01057 LINC01058
LINC01059 LINC00106 LINC01060 LINC01061 LINC01063 LINC01065 LINC01068 LINC01069 LINC01070
LINC01072 LINC01075 LINC01080 LINC01081 LINC01082 LINC01085 LINC01087 LINC01088 LINC01089
LINC01090 LINC01091 LINC01093 LINC01094 LINC01095 LINC01096 LINC01097 LINC01098 LINC01099
LINC01100 LINC01101 LINC01102 LINC01103 LINC01104 LINC01105 LINC01106 LINC01107 LINC01108
LINC01109 LINC00111 LINC01111 LINC01114 LINC01115 LINC01116 LINC01117 LINC01118 LINC01119
LINC00112 LINC01120 LINC01121 LINC01122 LINC01123 LINC01124 LINC01125 LINC01126 LINC01127
LINC01128 LINC00113 LINC01132 LINC01133 LINC01134 LINC01135 LINC01136 LINC01137 LINC01138
LINC01139 LINC00114 LINC01140 LINC01141 LINC01142 LINC01143 LINC01144 LINC01146 LINC01149
LINC00115 LINC01150 LINC01151 LINC01152 LINC01153 LINC01158 LINC01159 LINC00116 LINC01160
LINC01162 LINC01163 LINC01164 LINC01166 LINC01167 LINC01168 LINC01169 LINC01170 LINC01176
LINC01177 LINC01179 LINC01180 LINC01182 LINC01184 LINC01185 LINC01186 LINC01187 LINC01189
LINC01191 LINC01192 LINC01193 LINC01194 LINC01195 LINC01197 LINC01198 LINC01201 LINC01202
LINC01203 LINC01204 LINC01205 LINC01206 LINC01207 LINC01208 LINC01209 LINC01210 LINC01212
LINC01213 LINC01214 LINC01215 LINC01216 LINC01217 LINC01218 LINC01219 LINC01220 LINC01221
LINC01222 LINC01224 LINC01226 LINC01227 LINC01228 LINC01229 LINC01230 LINC01231 LINC01232
LINC01233 LINC01234 LINC01235 LINC01237 LINC01239 LINC01241 LINC01242 LINC01243 LINC01246
LINC01247 LINC01248 LINC01249 LINC01250 LINC01251 LINC01252 LINC01254 LINC01255 LINC01256
LINC01257 LINC01258 LINC01260 LINC01262 LINC01264 LINC01265 LINC01266 LINC01267 LINC01268
LINC01269 LINC01270 LINC01271 LINC01273 LINC01276 LINC01277 LINC01278 LINC01279 LINC01280
LINC01281 LINC01282 LINC01284 LINC01285 LINC01287 LINC01288 LINC01289 LINC01291 LINC01293
LINC01296 LINC01298 LINC01299 LINC01300 LINC01301 LINC01304 LINC01305 LINC01307 LINC01309
LINC01310 LINC01311 LINC01312 LINC01314 LINC01315 LINC01317 LINC01320 LINC01322 LINC01324
LINC01327 LINC01331 LINC01333 LINC01335 LINC01336 LINC01337 LINC01338 LINC01339 LINC01340
LINC01341 LINC01342 LINC01343 LINC01344 LINC01346 LINC01347 LINC01348 LINC01349 LINC01350
LINC01351 LINC01352 LINC01353 LINC01354 LINC01355 LINC01356 LINC01358 LINC01359 LINC01360
LINC01361 LINC01362 LINC01363 LINC01364 LINC01365 LINC01366 LINC01370 LINC01372 LINC01375
LINC01377 LINC01378 LINC01384 LINC01386 LINC01387 LINC01389 LINC01391 LINC01392 LINC01393
LINC01395 LINC01396 LINC01397 LINC01398 LINC01399 LINC01402 LINC01405 LINC01410 LINC01411
LINC01412 LINC01413 LINC01416 LINC01419 LINC01420 LINC01422 LINC01423 LINC01424 LINC01425
LINC01426 LINC01427 LINC01428 LINC01429 LINC01430 LINC01431 LINC01432 LINC01433 LINC01435
LINC01436 LINC01440 LINC01441 LINC01442 LINC01443 LINC01444 LINC01445 LINC01446 LINC01447
LINC01448 LINC01449 LINC01450 LINC01452 LINC01455 LINC01461 LINC01465 LINC01467 LINC01468
LINC01470 LINC01471 LINC01473 LINC01474 LINC01475 LINC01476 LINC01477 LINC01478 LINC01479
LINC01480 LINC01481 LINC01482 LINC01483 LINC01484 LINC01485 LINC01486 LINC01487 LINC01488
LINC01489 LINC01490 LINC01491 LINC01492 LINC01493 LINC01494 LINC01495 LINC01496 LINC01497
LINC01498 LINC01499 LINC01500 LINC01501 LINC01502 LINC01503 LINC01504 LINC01505 LINC01506
LINC01507 LINC01508 LINC01509 LINC01510 LINC01511 LINC01512 LINC01514 LINC01515 LINC01516
LINC01517 LINC01518 LINC01519 LINC00152 LINC01520 LINC01521 LINC01522 LINC01523 LINC01524
LINC01525 LINC01526 LINC01527 LINC01529 LINC01530 LINC01531 LINC01532 LINC01533 LINC01534
LINC01535 LINC01537 LINC01538 LINC01539 LINC01541 LINC01543 LINC01544 LINC01545 LINC01546
LINC01547 LINC01548 LINC01549 LINC01550 LINC01551 LINC01552 LINC01553 LINC01554 LINC01555
LINC01556 LINC01558 LINC01559 LINC01560 LINC01561 LINC01562 LINC01563 LINC01564 LINC01565
LINC01566 LINC01567 LINC01568 LINC01569 LINC01570 LINC01571 LINC01572 LINC01573 LINC01574
LINC01578 LINC01579 LINC00158 LINC01580 LINC01581 LINC01582 LINC01583 LINC01584 LINC01585
LINC01586 LINC01587 LINC01588 LINC01589 LINC00159 LINC01590 LINC01591 LINC01592 LINC01594
LINC01599 LINC00160 LINC01600 LINC01601 LINC01602 LINC01603 LINC01604 LINC01605 LINC01606
LINC01607 LINC01608 LINC01609 LINC00161 LINC00162 LINC00163 LINC00165 LINC00167 LINC00173
LINC00174 LINC00176 LINC00184 LINC00189 LINC00200 LINC00202-1 LINC00202-2 LINC00205
LINC00207 LINC00208 LINC00210 LINC00211 LINC00216 LINC00221 LINC00222 LINC00226 LINC00229
LINC00235 LINC00237 LINC00238 LINC00239 LINC00240 LINC00242 LINC00243 LINC00244 LINC00251
LINC00254 LINC00260 LINC00261 LINC00264 LINC00265 LINC00266-1 LINC00266-3 LINC00268
LINC00269 LINC00271 LINC00272 LINC00273 LINC00276 LINC00278 LINC00279 LINC00028 LINC00280
LINC00282 LINC00283 LINC00284 LINC00029 LINC00290 LINC00293 LINC00294 LINC00297 LINC00298
LINC00299 LINC00301 LINC00303 LINC00304 LINC00305 LINC00307 LINC00308 LINC00309 LINC00310
LINC00311 LINC00312 LINC00313 LINC00314 LINC00315 LINC00316 LINC00317 LINC00319 LINC00032
LINC00320 LINC00322 LINC00323 LINC00324 LINC00326 LINC00327 LINC00330 LINC00331 LINC00332
LINC00333 LINC00336 LINC00337 LINC00339 LINC00341 LINC00342 LINC00343 LINC00345 LINC00346
LINC00347 LINC00348 LINC00349 LINC00350 LINC00351 LINC00352 LINC00353 LINC00354 LINC00358
LINC00359 LINC00362 LINC00363 LINC00364 LINC00365 LINC00366 LINC00367 LINC00368 LINC00370
LINC00371 LINC00375 LINC00376 LINC00377 LINC00378 LINC00379 LINC00380 LINC00381 LINC00382
LINC00383 LINC00384 LINC00387 LINC00388 LINC00391 LINC00392 LINC00393 LINC00395 LINC00396
LINC00397 LINC00398 LINC00399 LINC00400 LINC00402 LINC00403 LINC00404 LINC00408 LINC00410
LINC00411 LINC00412 LINC00415 LINC00417 LINC00421 LINC00423 LINC00424 LINC00426 LINC00427
LINC00428 LINC00431 LINC00433 LINC00434 LINC00437 LINC00440 LINC00441 LINC00442 LINC00443
LINC00444 LINC00445 LINC00446 LINC00448 LINC00454 LINC00456 LINC00457 LINC00458 LINC00459
LINC00460 LINC00461 LINC00462 LINC00463 LINC00466 LINC00467 LINC00469 LINC00470 LINC00471
LINC00472 LINC00473 LINC00474 LINC00475 LINC00476 LINC00477 LINC00479 LINC00482 LINC00483
LINC00485 LINC00486 LINC00487 LINC00488 LINC00489 LINC00491 LINC00492 LINC00493 LINC00494
LINC00498 LINC00499 LINC00501 LINC00502 LINC00504 LINC00505 LINC00506 LINC00507 LINC00508
LINC00051 LINC00511 LINC00514 LINC00515 LINC00518 LINC00052 LINC00520 LINC00521 LINC00523
LINC00524 LINC00525 LINC00526 LINC00527 LINC00528 LINC00529 LINC00534 LINC00535 LINC00536
LINC00537 LINC00538 LINC00539 LINC00540 LINC00544 LINC00545 LINC00547 LINC00548 LINC00550
LINC00551 LINC00552 LINC00554 LINC00555 LINC00556 LINC00557 LINC00558 LINC00559 LINC00560
LINC00561 LINC00562 LINC00563 LINC00564 LINC00565 LINC00566 LINC00570 LINC00571 LINC00572
LINC00574 LINC00575 LINC00577 LINC00578 LINC00581 LINC00582 LINC00583 LINC00587 LINC00588
LINC00589 LINC00592 LINC00593 LINC00595 LINC00596 LINC00597 LINC00598 LINC00599 LINC00601
LINC00602 LINC00603 LINC00605 LINC00606 LINC00607 LINC00608 LINC00609 LINC00610 LINC00612
LINC00613 LINC00614 LINC00615 LINC00616 LINC00618 LINC00619 LINC00620 LINC00621 LINC00622
LINC00623 LINC00624 LINC00626 LINC00628 LINC00629 LINC00630 LINC00632 LINC00633 LINC00634
LINC00635 LINC00636 LINC00637 LINC00638 LINC00639 LINC00640 LINC00641 LINC00642 LINC00643
LINC00644 LINC00645 LINC00648 LINC00649 LINC00652 LINC00654 LINC00656 LINC00657 LINC00658
LINC00659 LINC00661 LINC00662 LINC00663 LINC00664 LINC00665 LINC00667 LINC00668 LINC00669
LINC00670 LINC00671 LINC00672 LINC00673 LINC00674 LINC00675 LINC00676 LINC00677 LINC00678
LINC00681 LINC00682 LINC00683 LINC00684 LOC100132304 LINC00685 LINC00686 LINC00687
LINC00689 LINC00690 LINC00691 LINC00692 LINC00693 LINC00694 LINC00696 LINC00698 LINC00700
LINC00701 LINC00702 LINC00703 LINC00704 LINC00705 LINC00706 LINC00707 LINC00708 LINC00709
LINC00710 LINC00824 LINC00836 LINC00837 LINC00838 LINC00839 LINC00840 LINC00841 LINC00842
LINC00843 LINC00844 LINC00845 LINC00847 LINC00849 LINC00850 LINC00851 LINC00852 LINC00853
LINC00854 LINC00856 LINC00857 LINC00858 LINC00861 LINC00862 LINC00864 LINC00865 LINC00866
LINC00867 LINC00869 LINC00870 LINC00871 LINC00877 LINC00879 LINC00880 LINC00881 LINC00882
LINC00883 LINC00884 LINC00885 LINC00886 LINC00887 LINC00888 LINC00889 LINC00890 LINC00891
LINC00892 LINC00893 LINC00894 LINC00895 LINC00896 LINC00898 LINC00899 LINC00900 LINC00901
LINC00903 LINC00904 LINC00905 LINC00906 LINC00907 LINC00908 LINC00909 LINC00910 LINC00911
LINC00914 LINC00917 LINC00919 LINC00092 LINC00920 LINC00921 LINC00922 LINC00923 LINC00924
LINC00925 LINC00926 LINC00927 LINC00928 LINC00929 LINC00930 LINC00933 LINC00934 LINC00935
LINC00936 LINC00937 LINC00938 LINC00939 LINC00094 LINC00940 LINC00941 LINC00942 LINC00943
LINC00944 LINC00945 LINC00950 LINC00951 LINC00954 LINC00955 LINC00957 LINC00958 LINC00959
LINC00960 LINC00961 LINC00963 LINC00964 LINC00965 LINC00967 LINC00968 LINC00969 LINC00970
LINC00971 LINC00972 LINC00973 LINC00974 LINC00977 LINC00982 LINC00987 LINC00989 LINC00992
LINC00993 LINC00994 LINC00996 LINC00997 LINC00998 LINC00999 PNKY LINCMD1 LINC-PINT LINC-
ROR LOH12CR2 LOXL1-AS1 LPP-AS1 LPP-AS2 LRP4-AS1 LRRC2-AS1 LRRC3-AS1 LRRC3DN LRRC75A-
AS1 LSAMP-AS1 LACAT1 LUCAT1 LCA10 LURAP1L-AS1 LY86-AS1 LYPLAL1-AS1 LYST-AS1 LZTS1-
AS1 MACC1-AS1 MACROD2-AS1 MACROD2-IT1 MAFTRR MAFA-AS1 MAFG-AS1 MAGEA8-AS1
MAGI1-AS1 MAGI2-AS1 MAGI2-AS2 MAGI2-AS3 MAMDC2-AS1 MAN1B1-AS1 MANEA-AS1 MAP3K14-
AS1 MAPKAPK5-AS1 MAPT-AS1 MAPT-IT1 MAST4-AS1 MAST4-IT1 MEG3 MEG8 MEG9 MATN1-AS1
MBNL1-AS1 MCF2L-AS1 MCHR2-AS1 MCM3AP-AS1 MCM8-AS1 MCPH1-AS1 MED4-AS1 MEF2C-AS1
MEIS1-AS2 MEIS1-AS3 MEAT6 MEOX2-AS1 MIMT1 MESTIT1 MALAT1 MFI2-AS1 MGAT3-AS1 MIA-
RAB4B MIATNB MIR100 MIR101-1 MIR101-2 MIR103A1 MIR103A2 MIR103B1 MIR103B2 MIR105-1
MIR105-2 MIR106A MIR106B MIR107 MIR10A MIR10B MIR1-1 MIR1178 MIR1179 MIR1180 MIR1181
MIR1182 MIR1183 MIR1184-1 MIR1184-2 MIR1184-3 MIR1185-1 MIR1185-2 MIR1193 MIR1197 MIR1199
MIR1-2 MIR1200 MIR1202 MIR1203 MIR1204 MIR1205 MIR1206 MIR1207 MIR1208 MIR122 MIR1224
MIR1225 MIR1226 MIR1227 MIR1228 MIR1229 MIR1231 MIR1233-1 MIR1233-2 MIR1234 MIR1236
MIR1237 MIR1238 MIR124-1 MIR124-2 MIR1243 MIR124-3 MIR1244-1 MIR1244-2 MIR1244-3 MIR1244-4
MIR1245A MIR1245B MIR1246 MIR1247 MIR1248 MIR1249 MIR1250 MIR1251 MIR1252 MIR1253
MIR1254-1 MIR1254-2 MIR1255A MIR1255B1 MIR1255B2 MIR1256 MIR1257 MIR1258 MIR125A MIR125B1
MIR125B2 MIR126 MIR1260A MIR1260B MIR1261 MIR1262 MIR1263 MIR1264 MIR1265 MIR1266
MIR1267 MIR1268A MIR1268B MIR1269A MIR1269B MIR127 MIR1270 MIR1271 MIR1272 MIR1273A
MIR1273C MIR1273D MIR1273E MIR1273F MIR1273G MIR1273H MIR1275 MIR1276 MIR1277 MIR1278
MIR1279 MIR1281 MIR128-1 MIR1282 MIR128-2 MIR1283-1 MIR1283-2 MIR1284 MIR1285-1 MIR1285-2
MIR1286 MIR1287 MIR1288 MIR1289-1 MIR1289-2 MIR1290 MIR1291 MIR129-1 MIR1292 MIR129-2
MIR1293 MIR1294 MIR1295A MIR1295B MIR1296 MIR1297 MIR1298 MIR1299 MIR1301 MIR1302-1
MIR1302-10 MIR1302-11 MIR1302-2 MIR1302-3 MIR1302-4 MIR1302-5 MIR1302-6 MIR1302-7 MIR1302-8
MIR1302-9 MIR1303 MIR1304 MIR1305 MIR1306 MIR1307 MIR130A MIR130B MIR132 MIR1321 MIR1322
MIR1323 MIR1324 MIR133A1 MIR133A2 MIR133B MIR134 MIR1343 MIR135A1 MIR135A2 MIR135B
MIR136 MIR137 MIR138-1 MIR138-2 MIR139 MIR140 MIR141 MIR142 MIR143 MIR144 MIR145 MIR1468
MIR1469 MIR146A MIR146B MIR1470 MIR1471 MIR147A MIR147B MIR148A MIR148B MIR149 MIR150
MIR151A MIR151B MIR152 MIR153-1 MIR153-2 MIR1537 MIR1538 MIR1539 MIR154 MIR155 MIR1587
MIR15A MIR15B MIR16-1 MIR16-2 MIR17 MIR181A1 MIR181A2 MIR181B1 MIR181B2 MIR181C MIR181D
MIR182 MIR1825 MIR1827 MIR183 MIR184 MIR185 MIR186 MIR187 MIR188 MIR18A MIR18B MIR1908
MIR1909 MIR190A MIR190B MIR191 MIR1910 MIR1911 MIR1912 MIR1913 MIR1914 MIR1915 MIR192
MIR193A MIR193B MIR194-1 MIR194-2 MIR195 MIR196A1 MIR196A2 MIR196B MIR197 MIR1972-1
MIR1972-2 MIR1973 MIR1976 MIR198 MIR199A1 MIR199A2 MIR199B MIR19A MIR19B1 MIR19B2
MIR200A MIR200B MIR200C MIR202 MIR203A MIR203B MIR204 MIR205 MIR2052 MIR2053 MIR2054
MIR206 MIR208A MIR208B MIR20A MIR20B MIR21 MIR210 MIR211 MIR2110 MIR2113 MIR2114 MIR2115
MIR2116 MIR2117 MIR212 MIR214 MIR215 MIR216A MIR216B MIR217 MIR218-1 MIR218-2 MIR219A1
MIR219A2 MIR219B MIR22 MIR221 MIR222 MIR223 MIR224 MIR2276 MIR2277 MIR2278 MIR2355
MIR2392 MIR23A MIR23B MIR23C MIR24-1 MIR24-2 MIR2467 MIR25 MIR2681 MIR2682 MIR26A1
MIR26A2 MIR26B MIR27A MIR27B MIR28 MIR2861 MIR2909 MIR296 MIR297 MIR298 MIR299 MIR29A
MIR29B1 MIR29B2 MIR29C MIR300 MIR301A MIR301B MIR302A MIR302B MIR302C MIR302D MIR302E
MIR302F MIR3064 MIR3065 MIR3074 MIR30A MIR30B MIR30C1 MIR30C2 MIR30D MIR30E MIR31
MIR3115 MIR3116-1 MIR3116-2 MIR3117 MIR3118-1 MIR3118-2 MIR3118-3 MIR3118-4 MIR3119-1
MIR3119-2 MIR3120 MIR3121 MIR3122 MIR3123 MIR3124 MIR3125 MIR3126 MIR3127 MIR3128 MIR3129
MIR3130-1 MIR3130-2 MIR3131 MIR3132 MIR3133 MIR3134 MIR3135A MIR3135B MIR3136 MIR3137
MIR3138 MIR3139 MIR3140 MIR3141 MIR3142 MIR3143 MIR3144 MIR3145 MIR3146 MIR3147 MIR3148
MIR3149 MIR3150A MIR3150B MIR3151 MIR3152 MIR3153 MIR3154 MIR3155A MIR3155B MIR3156-1
MIR3156-2 MIR3156-3 MIR3157 MIR3158-1 MIR3158-2 MIR3159 MIR3160-1 MIR3160-2 MIR3161 MIR3162
MIR3163 MIR3164 MIR3165 MIR3166 MIR3167 MIR3168 MIR3169 MIR3170 MIR3171 MIR3173 MIR3174
MIR3175 MIR3176 MIR3177 MIR3178 MIR3179-1 MIR3179-2 MIR3179-3 MIR3179-4 MIR3180-1 MIR3180-2
MIR3180-3 MIR3180-4 MIR3180-5 MIR3181 MIR3182 MIR3183 MIR3184 MIR3185 MIR3186 MIR3187
MIR3188 MIR3189 MIR3190 MIR3191 MIR3192 MIR3193 MIR3194 MIR3195 MIR3196 MIR3197 MIR3198-1
MIR3198-2 MIR3199-1 MIR3199-2 MIR32 MIR3200 MIR3201 MIR3202-1 MIR3202-2 MIR320A MIR320B1
MIR320B2 MIR320C1 MIR320C2 MIR320D1 MIR320D2 MIR320E MIR323A MIR323B MIR324 MIR325
MIR326 MIR328 MIR329-1 MIR329-2 MIR330 MIR331 MIR335 MIR337 MIR338 MIR339 MIR33A MIR33B
MIR340 MIR342 MIR345 MIR346 MIR34A MIR34B MIR34C MIR3529 MIR3591 MIR3605 MIR3606 MIR3607
MIR3609 MIR361 MIR3610 MIR3611 MIR3612 MIR3613 MIR3614 MIR3615 MIR3616 MIR3617 MIR3618
MIR3619 MIR362 MIR3620 MIR3621 MIR3622A MIR3622B MIR363 MIR3646 MIR3648-1 MIR3648-2
MIR3649 MIR3650 MIR3651 MIR3652 MIR3653 MIR3654 MIR3655 MIR3656 MIR3657 MIR3658 MIR3659
MIR365A MIR365B MIR3660 MIR3661 MIR3662 MIR3663 MIR3664 MIR3665 MIR3666 MIR3667 MIR3668
MIR367 MIR3670-1 MIR3670-2 MIR3670-3 MIR3670-4 MIR3671 MIR3672 MIR3674 MIR3675 MIR3677
MIR3678 MIR3679 MIR3680-1 MIR3680-2 MIR3681 MIR3682 MIR3683 MIR3684 MIR3685 MIR3686
MIR3687-1 MIR3687-2 MIR3688-1 MIR3688-2 MIR3689A MIR3689B MIR3689C MIR3689D1 MIR3689D2
MIR3689E MIR3689F MIR369 MIR3690 MIR3691 MIR3692 MIR370 MIR3713 MIR3714 MIR371A MIR371B
MIR372 MIR373 MIR374A MIR374B MIR374C MIR375 MIR376A1 MIR376A2 MIR376B MIR376C MIR377
MIR378A MIR378B MIR378C MIR378D1 MIR378D2 MIR378E MIR378F MIR378G MIR378H MIR378I
MIR378J MIR379 MIR380 MIR381 MIR382 MIR383 MIR384 MIR3907 MIR3908 MIR3909 MIR3910-1
MIR3910-2 MIR3911 MIR3912 MIR3913-1 MIR3913-2 MIR3914-1 MIR3914-2 MIR3915 MIR3916 MIR3917
MIR3918 MIR3919 MIR3920 MIR3921 MIR3922 MIR3923 MIR3924 MIR3925 MIR3926-1 MIR3926-2
MIR3927 MIR3928 MIR3929 MIR3934 MIR3935 MIR3936 MIR3937 MIR3938 MIR3939 MIR3940 MIR3941
MIR3942 MIR3943 MIR3944 MIR3945 MIR3960 MIR3972 MIR3973 MIR3974 MIR3975 MIR3976 MIR3977
MIR3978 MIR409 MIR410 MIR411 MIR412 MIR421 MIR422A MIR423 MIR424 MIR425 MIR4251 MIR4252
MIR4253 MIR4254 MIR4255 MIR4256 MIR4257 MIR4258 MIR4259 MIR4260 MIR4261 MIR4262 MIR4263
MIR4264 MIR4265 MIR4266 MIR4267 MIR4268 MIR4269 MIR4270 MIR4271 MIR4272 MIR4273 MIR4274
MIR4275 MIR4276 MIR4277 MIR4278 MIR4279 MIR4280 MIR4281 MIR4282 MIR4283-1 MIR4283-2
MIR4284 MIR4285 MIR4286 MIR4287 MIR4288 MIR4289 MIR429 MIR4290 MIR4291 MIR4292 MIR4293
MIR4294 MIR4295 MIR4296 MIR4297 MIR4298 MIR4299 MIR4300 MIR4301 MIR4302 MIR4303 MIR4304
MIR4305 MIR4306 MIR4307 MIR4308 MIR4309 MIR431 MIR4310 MIR4311 MIR4312 MIR4313 MIR4314
MIR4315-1 MIR4315-2 MIR4316 MIR4317 MIR4318 MIR4319 MIR432 MIR4320 MIR4321 MIR4322 MIR4323
MIR4324 MIR4325 MIR4326 MIR4327 MIR4328 MIR4329 MIR433 MIR4330 MIR4417 MIR4418 MIR4419A
MIR4419B MIR4420 MIR4421 MIR4422 MIR4423 MIR4424 MIR4425 MIR4426 MIR4427 MIR4428 MIR4429
MIR4430 MIR4431 MIR4432 MIR4433A MIR4433B MIR4434 MIR4435-1 MIR4435-2 MIR4436A MIR4436B1
MIR4436B2 MIR4437 MIR4438 MIR4439 MIR4440 MIR4441 MIR4442 MIR4443 MIR4444-1 MIR4444-2
MIR4445 MIR4446 MIR4447 MIR4448 MIR4449 MIR4450 MIR4451 MIR4452 MIR4453 MIR4454 MIR4455
MIR4456 MIR4457 MIR4458 MIR4459 MIR4460 MIR4461 MIR4462 MIR4463 MIR4464 MIR4465 MIR4466
MIR4467 MIR4468 MIR4469 MIR4470 MIR4471 MIR4472-1 MIR4472-2 MIR4473 MIR4474 MIR4475
MIR4476 MIR4477A MIR4477B MIR4478 MIR4479 MIR448 MIR4480 MIR4481 MIR4482 MIR4483 MIR4484
MIR4485 MIR4486 MIR4487 MIR4488 MIR4489 MIR4490 MIR4491 MIR4492 MIR4493 MIR4494 MIR4495
MIR4496 MIR4497 MIR4498 MIR4499 MIR449A MIR449B MIR449C MIR4500 MIR4501 MIR4502 MIR4503
MIR4504 MIR4505 MIR4506 MIR4507 MIR4508 MIR4509-1 MIR4509-2 MIR4509-3 MIR450A1 MIR450A2
MIR450B MIR4510 MIR4511 MIR4512 MIR4513 MIR4514 MIR4515 MIR4516 MIR4517 MIR4518 MIR4519
MIR451A MIR451B MIR452 MIR4520-1 MIR4520-2 MIR4521 MIR4522 MIR4523 MIR4524A MIR4524B
MIR4525 MIR4526 MIR4527 MIR4528 MIR4529 MIR4530 MIR4531 MIR4532 MIR4533 MIR4534 MIR4535
MIR4536-1 MIR4536-2 MIR4537 MIR4538 MIR4539 MIR454 MIR4540 MIR455 MIR4632 MIR4633 MIR4634
MIR4635 MIR4636 MIR4637 MIR4638 MIR4639 MIR4640 MIR4641 MIR4642 MIR4643 MIR4644 MIR4645
MIR4646 MIR4647 MIR4648 MIR4649 MIR4650-1 MIR4650-2 MIR4651 MIR4652 MIR4653 MIR4654
MIR4655 MIR4656 MIR4657 MIR4658 MIR4659A MIR4659B MIR466 MIR4660 MIR4661 MIR4662A
MIR4662B MIR4663 MIR4664 MIR4665 MIR4666A MIR4666B MIR4667 MIR4668 MIR4669 MIR4670
MIR4671 MIR4672 MIR4673 MIR4674 MIR4675 MIR4676 MIR4677 MIR4678 MIR4679-1 MIR4679-2
MIR4680 MIR4681 MIR4682 MIR4683 MIR4684 MIR4685 MIR4686 MIR4687 MIR4688 MIR4689 MIR4690
MIR4691 MIR4692 MIR4693 MIR4694 MIR4695 MIR4696 MIR4697 MIR4698 MIR4699 MIR4700 MIR4701
MIR4703 MIR4704 MIR4705 MIR4706 MIR4707 MIR4708 MIR4709 MIR4710 MIR4711 MIR4712 MIR4713
MIR4714 MIR4715 MIR4716 MIR4717 MIR4718 MIR4719 MIR4720 MIR4721 MIR4722 MIR4723 MIR4724
MIR4725 MIR4726 MIR4727 MIR4728 MIR4729 MIR4730 MIR4731 MIR4732 MIR4733 MIR4734 MIR4735
MIR4736 MIR4737 MIR4738 MIR4739 MIR4740 MIR4741 MIR4742 MIR4743 MIR4744 MIR4745 MIR4746
MIR4747 MIR4748 MIR4749 MIR4750 MIR4751 MIR4752 MIR4753 MIR4754 MIR4755 MIR4756 MIR4757
MIR4758 MIR4759 MIR4760 MIR4761 MIR4762 MIR4763 MIR4764 MIR4765 MIR4766 MIR4767 MIR4768
MIR4769 MIR4770 MIR4771-1 MIR4771-2 MIR4772 MIR4773-1 MIR4773-2 MIR4774 MIR4775 MIR4776-1
MIR4776-2 MIR4777 MIR4778 MIR4779 MIR4780 MIR4781 MIR4782 MIR4783 MIR4784 MIR4785 MIR4786
MIR4787 MIR4788 MIR4789 MIR4790 MIR4791 MIR4792 MIR4793 MIR4794 MIR4795 MIR4796 MIR4797
MIR4798 MIR4799 MIR4800 MIR4801 MIR4802 MIR4803 MIR4804 MIR483 MIR484 MIR485 MIR486-1
MIR486-2 MIR487A MIR487B MIR488 MIR489 MIR490 MIR491 MIR492 MIR493 MIR494 MIR495 MIR496
MIR497 MIR498 MIR4999 MIR499A MIR499B MIR5000 MIR5001 MIR5002 MIR5003 MIR5004 MIR5006
MIR5007 MIR5008 MIR5009 MIR500A MIR500B MIR501 MIR5010 MIR5011 MIR502 MIR503 MIR504
MIR5047 MIR505 MIR506 MIR507 MIR508 MIR5087 MIR5088 MIR5089 MIR5090 MIR5091 MIR509-1
MIR5092 MIR509-2 MIR5093 MIR509-3 MIR5094 MIR5095 MIR5096 MIR510 MIR5100 MIR511 MIR512-1
MIR512-2 MIR513A1 MIR513A2 MIR513B MIR513C MIR514A1 MIR514A2 MIR514A3 MIR514B MIR515-1
MIR515-2 MIR516A1 MIR516A2 MIR516B1 MIR516B2 MIR517A MIR517B MIR517C MIR5186 MIR5187
MIR5188 MIR5189 MIR518A1 MIR518A2 MIR518B MIR518C MIR518D MIR518E MIR518F MIR5190
MIR5191 MIR5192 MIR5193 MIR5194 MIR5195 MIR5196 MIR5197 MIR519A1 MIR519A2 MIR519B
MIR519C MIR519D MIR519E MIR520A MIR520B MIR520C MIR520D MIR520E MIR520F MIR520G
MIR520H MIR521-1 MIR521-2 MIR522 MIR523 MIR524 MIR525 MIR526A1 MIR526A2 MIR526B MIR527
MIR532 MIR539 MIR541 MIR542 MIR543 MIR544A MIR544B MIR545 MIR548A1 MIR548A2 MIR548A3
MIR548AA1 MIR548AA2 MIR548AB MIR548AC MIR548AD MIR548AE1 MIR548AE2 MIR548AG1
MIR548AG2 MIR548AH MIR548AI MIR548AJ1 MIR548AJ2 MIR548AK MIR548AL MIR548AM MIR548AN
MIR548AO MIR548AP MIR548AQ MIR548AR MIR548AS MIR548AT MIR548AU MIR548AV MIR548AW
MIR548AX MIR548AY MIR548AZ MIR548B MIR548BA MIR548BB MIR548C MIR548D1 MIR548D2
MIR548E MIR548F1 MIR548F2 MIR548F3 MIR548F4 MIR548F5 MIR548G MIR548H1 MIR548H2 MIR548H3
MIR548H4 MIR548H5 MIR548I1 MIR548I2 MIR548I3 MIR548I4 MIR548J MIR548K MIR548L MIR548M
MIR548N MIR548O MIR548O2 MIR548P MIR548Q MIR548S MIR548T MIR548U MIR548V MIR548W
MIR548X MIR548X2 MIR548Y MIR548Z MIR549A MIR550A1 MIR550A2 MIR550A3 MIR550B1 MIR550B2
MIR551A MIR551B MIR552 MIR553 MIR554 MIR555 MIR556 MIR557 MIR5571 MIR5572 MIR5579 MIR558
MIR5580 MIR5581 MIR5582 MIR5583-1 MIR5583-2 MIR5584 MIR5585 MIR5586 MIR5587 MIR5588
MIR5589 MIR559 MIR5590 MIR5591 MIR561 MIR562 MIR563 MIR564 MIR566 MIR567 MIR568 MIR5680
MIR5681A MIR5681B MIR5682 MIR5683 MIR5684 MIR5685 MIR5687 MIR5688 MIR5689 MIR569 MIR5690
MIR5691 MIR5692A1 MIR5692A2 MIR5692B MIR5692C1 MIR5692C2 MIR5693 MIR5694 MIR5695 MIR5696
MIR5697 MIR5698 MIR5699 MIR570 MIR5700 MIR5701-1 MIR5701-2 MIR5701-3 MIR5702 MIR5703
MIR5704 MIR5705 MIR5706 MIR5707 MIR5708 MIR571 MIR572 MIR573 MIR5739 MIR574 MIR575 MIR576
MIR577 MIR578 MIR5787 MIR579 MIR580 MIR581 MIR582 MIR583 MIR584 MIR585 MIR586 MIR587
MIR588 MIR589 MIR590 MIR591 MIR592 MIR593 MIR595 MIR596 MIR597 MIR598 MIR599 MIR600
MIR601 MIR602 MIR603 MIR604 MIR605 MIR606 MIR6068 MIR6069 MIR607 MIR6070 MIR6071 MIR6072
MIR6073 MIR6074 MIR6075 MIR6076 MIR6077 MIR6078 MIR6079 MIR608 MIR6080 MIR6081 MIR6082
MIR6083 MIR6084 MIR6085 MIR6086 MIR6087 MIR6088 MIR6089 MIR609 MIR6090 MIR610 MIR611
MIR612 MIR6124 MIR6125 MIR6126 MIR6127 MIR6128 MIR6129 MIR613 MIR6130 MIR6131 MIR6132
MIR6133 MIR6134 MIR614 MIR615 MIR616 MIR6165 MIR617 MIR618 MIR619 MIR620 MIR621 MIR622
MIR623 MIR624 MIR625 MIR626 MIR627 MIR628 MIR629 MIR630 MIR631 MIR632 MIR633 MIR634
MIR635 MIR636 MIR637 MIR638 MIR639 MIR640 MIR641 MIR642A MIR642B MIR643 MIR644A MIR645
MIR646 MIR647 MIR648 MIR649 MIR6499 MIR650 MIR6500 MIR6501 MIR6502 MIR6503 MIR6504
MIR6505 MIR6506 MIR6507 MIR6508 MIR6509 MIR651 MIR6510 MIR6511A1 MIR6511A2 MIR6511A3
MIR6511A4 MIR6511B1 MIR6511B2 MIR6512 MIR6513 MIR6514 MIR6515 MIR6516 MIR652 MIR653
MIR654 MIR655 MIR656 MIR657 MIR658 MIR659 MIR660 MIR661 MIR662 MIR663A MIR663B MIR664A
MIR664B MIR665 MIR668 MIR670 MIR671 MIR6715A MIR6715B MIR6716 MIR6717 MIR6718 MIR6719
MIR6720 MIR6721 MIR6722 MIR6723 MIR6724-1 MIR6724-2 MIR6724-3 MIR6724-4 MIR6726 MIR6727
MIR6728 MIR6729 MIR6730 MIR6731 MIR6732 MIR6733 MIR6734 MIR6735 MIR6736 MIR6737 MIR6738
MIR6739 MIR6740 MIR6741 MIR6742 MIR6743 MIR6744 MIR6745 MIR6746 MIR6747 MIR6748 MIR6749
MIR675 MIR6750 MIR6751 MIR6752 MIR6753 MIR6754 MIR6755 MIR6756 MIR6757 MIR6758 MIR6759
MIR676 MIR6760 MIR6761 MIR6762 MIR6763 MIR6764 MIR6765 MIR6766 MIR6767 MIR6768 MIR6769A
MIR6769B MIR6770-1 MIR6770-2 MIR6770-3 MIR6771 MIR6772 MIR6773 MIR6774 MIR6775 MIR6776
MIR6777 MIR6778 MIR6779 MIR6780A MIR6780B MIR6781 MIR6782 MIR6783 MIR6784 MIR6785 MIR6786
MIR6787 MIR6788 MIR6789 MIR6790 MIR6791 MIR6792 MIR6793 MIR6794 MIR6795 MIR6796 MIR6797
MIR6798 MIR6799 MIR6800 MIR6801 MIR6802 MIR6803 MIR6804 MIR6805 MIR6806 MIR6807 MIR6808
MIR6809 MIR6810 MIR6811 MIR6812 MIR6813 MIR6814 MIR6815 MIR6816 MIR6817 MIR6818 MIR6819
MIR6820 MIR6821 MIR6822 MIR6823 MIR6824 MIR6825 MIR6826 MIR6827 MIR6828 MIR6829 MIR6830
MIR6831 MIR6832 MIR6833 MIR6834 MIR6835 MIR6836 MIR6837 MIR6838 MIR6839 MIR6840 MIR6841
MIR6842 MIR6843 MIR6844 MIR6845 MIR6846 MIR6847 MIR6848 MIR6849 MIR6850 MIR6851 MIR6852
MIR6853 MIR6854 MIR6855 MIR6856 MIR6857 MIR6858 MIR6859-1 MIR6859-2 MIR6859-3 MIR6859-4
MIR6860 MIR6861 MIR6862-1 MIR6862-2 MIR6863 MIR6864 MIR6865 MIR6866 MIR6867 MIR6868
MIR6869 MIR6870 MIR6871 MIR6872 MIR6873 MIR6874 MIR6875 MIR6876 MIR6877 MIR6878 MIR6879
MIR6880 MIR6881 MIR6882 MIR6883 MIR6884 MIR6885 MIR6886 MIR6887 MIR6888 MIR6889 MIR6890
MIR6891 MIR6892 MIR6893 MIR6894 MIR6895 MIR708 MIR7-1 MIR7106 MIR7107 MIR7108 MIR7109
MIR711 MIR7110 MIR7111 MIR7112 MIR7113 MIR7114 MIR7150 MIR7151 MIR7152 MIR7153 MIR7154
MIR7155 MIR7156 MIR7157 MIR7158 MIR7159 MIR7160 MIR7161 MIR7162 MIR718 MIR7-2 MIR7-3
MIR744 MIR7515 MIR758 MIR759 MIR760 MIR761 MIR762 MIR764 MIR7641-1 MIR7641-2 MIR765 MIR766
MIR767 MIR769 MIR770 MIR7702 MIR7703 MIR7704 MIR7705 MIR7706 MIR7843 MIR7844 MIR7845
MIR7846 MIR7847 MIR7848 MIR7849 MIR7850 MIR7851 MIR7852 MIR7853 MIR7854 MIR7855 MIR7856
MIR7973-1 MIR7973-2 MIR7974 MIR7975 MIR7976 MIR7977 MIR7978 MIR802 MIR8052 MIR8053 MIR8054
MIR8055 MIR8056 MIR8057 MIR8058 MIR8059 MIR8060 MIR8061 MIR8062 MIR8063 MIR8064 MIR8065
MIR8066 MIR8067 MIR8068 MIR8069-1 MIR8069-2 MIR8070 MIR8071-1 MIR8071-2 MIR8072 MIR8073
MIR8074 MIR8075 MIR8076 MIR8077 MIR8078 MIR8079 MIR8080 MIR8081 MIR8082 MIR8083 MIR8084
MIR8085 MIR8086 MIR8087 MIR8088 MIR8089 MIR8485 MIR873 MIR874 MIR875 MIR876 MIR877 MIR885
MIR887 MIR888 MIR889 MIR890 MIR891A MIR891B MIR892A MIR892B MIR892C MIR9-1 MIR9-2 MIR920
MIR921 MIR922 MIR924 MIR92A1 MIR92A2 MIR92B MIR93 MIR9-3 MIR933 MIR934 MIR935 MIR936
MIR937 MIR938 MIR939 MIR940 MIR941-1 MIR941-2 MIR941-3 MIR941-4 MIR941-5 MIR942 MIR943
MIR944 MIR95 MIR9500 MIR96 MIR98 MIR99A MIR99B MIRLET7A1 MIRLET7A2 MIRLET7A3
MIRLET7B MIRLET7C MIRLET7D MIRLET7E MIRLET7F1 MIRLET7F2 MIRLET7G MIRLET7I MID1IP1-
AS1 MIF-AS1 MIR100HG MIR124-2HG MIR133A1HG MIR137HG MIR143HG MIR155HG MIR17HG
MIR181A1HG MIR181A2HG MIR202HG MIR2052HG MIR210HG MIR217HG MIR22HG MIR222HG
MIR31HG MIR325HG MIR3663HG MIR381HG MIR3976HG MIR4435-2HG MIR4458HG MIR4500HG
MIR4697HG MIR497HG MIR503HG MIR600HG MIR646HG MIR663AHG MIR670HG MIR7-3HG
MIR7515HG MIR762HG MIR99AHG MIRLET7BHG MIRLET7DHG MIS18A-AS1 MT-LIPCAR MKLN1-AS
MKNK1-AS1 MKRN3-AS1 MKX-AS1 MLIP-AS1 MLIP-IT1 MLK7-AS1 MLLT4-AS1 MME-AS1 MMP24-AS1
MMP25-AS1 MNX1-AS1 MORC1-AS1 MORC2-AS1 MORF4L2-AS1 MPRIP-AS1 MRGPRF-AS1 MRGPRG-
AS1 MROH7-TTC4 MRPL23-AS1 MRVI1-AS1 MSC-AS1 MSH5-SAPCD1 MTOR-AS1 MTUS2-AS1 MTUS2-
AS2 MYB-AS1 MYCBP2-AS1 MYCBP2-AS2 MYCNOS MYCNUT MDS2 MYLK-AS1 MYLK-AS2 MYO16-
AS1 MYO16-AS2 MIAT MYHAS MHRT MYT1L-AS1 MZF1-AS1 N4BP2L2-IT2 NAALADL2-AS1
NAALADL2-AS2 NAALADL2-AS3 NADK2-AS1 NAGPA-AS1 NALCN-AS1 NAPA-AS1 NARF-IT1 NAV2-
AS1 NAV2-AS2 NAV2-AS3 NAV2-AS4 NAV2-AS5 NAV2-IT1 NCAM1-AS1 NCBP2-AS1 NCBP2-AS2 NCK1-
AS1 NCKAP5-IT1 NCOA7-AS1 LOC104968399 NDFIP2-AS1 NDP-AS1 NDUFA6-AS1 NDUFB2-AS1
NDUFV2-AS1 NEBL-AS1 NRAV NRIR NEGR1-IT1 NBR2 NEURL1-AS1 NBAT1 NHEG1 NEXN-AS1 NFIA-
AS1 NFIA-AS2 NKILA NFYC-AS1 NHS-AS1 NIFK-AS1 NIPBL-AS1 NKX2-1-AS1 NKX2-2-AS1 NLGN1-AS1
NLGN4Y-AS1 NNT-AS1 NCRNA00250 NAMA NRON NCRUPAR NOP14-AS1 NPHP3-AS1 NPHP3-ACAD11
NPPA-AS1 NPSR1-AS1 NPTN-IT1 NR2F1-AS1 NR2F2-AS1 NREP-AS1 NRG1-IT1 NRG1-IT3 NRG3-AS1
NRSN2-AS1 NTM-IT NTRK3-AS1 NUCB1-AS1 NEAT1 NUP50-AS1 NUTM2A-AS1 NUTM2B-AS1 OCIAD1-
AS1 OGFR-AS1 OIP5-AS1 OLMALINC OPA1-AS1 OPCML-IT1 OPCML-IT2 OR2A1-AS1 OSBPL10-AS1
OSER1-AS1 OSGEPL1-AS1 OSMR-AS1 OTUD6B-AS1 OTX2-AS1 OVAAL OVCH1-AS1 OVOL1-AS1
OXCT1-AS1 P2RX5-TAX1BP3 P3H2-AS1 P4HA2-AS1 LOC104940763 PRG1 PABPC1L2B-AS1 PABPC5-AS1
PACRG-AS1 PAN3-AS1 PTCSC1 PTCSC2 PTCSC3 PAPPA-AS1 PAPPA-AS2 PAQR9-AS1 PARD3-AS1
PARD6G-AS1 PAUPAR PAX8-AS1 PAXBP1-AS1 PAXIP1-AS1 PAXIP1-AS2 PCBP1-AS1 PCBP2-OT1 PCCA-
AS1 PCDH9-AS1 PCDH9-AS2 PCDH9-AS3 PCDH9-AS4 PCED1B-AS1 PCGEM1 PCNA-AS1 PCOLCE-AS1
PCYT1B-AS1 PDCD4-AS1 PDX1-AS1 PDZRN3-AS1 PEG3-AS1 PEX5L-AS1 PEX5L-AS2 PGM5-AS1
PGM5P3-AS1 PGM5P4-AS1 PHEX-AS1 PHKA1-AS1 PHKA2-AS1 HPBP PGM5P3 PIK3CD-AS1 PIK3CD-AS2
PIK3IP1-AS1 PINK1-AS PIR-FIGF PITPNA-AS1 PITRM1-AS1 PKIA-AS1 PKN2-AS1 PKNOX2-AS1
PLA2G4E-AS1 PLBD1-AS1 PLCB1-IT1 PLCB2-AS1 PLCE1-AS1 PLCE1-AS2 PLCG1-AS1 PLCH1-AS1
PLCH1-AS2 PLCL2-AS1 PLCXD2-AS1 PLS1-AS1 PLS3-AS1 PLSCR5-AS1 PISRT1 POT1-AS1 POTEH-AS1
POU6F2-AS1 POU6F2-AS2 PPEF1-AS1 PPP1R26-AS1 PPP2R2B-IT1 PPP3CB-AS1 PPP4R1-AS1 PPT2-EGFL8
PWAR1 PWAR4 PWAR5 PWAR6 PWARSN PWRN1 PWRN2 PWRN3 PWRN4 PRC1-AS1 PRH1-PRR4
PRICKLE2-AS1 PRICKLE2-AS2 PRICKLE2-AS3 PRNT PRKAG2-AS1 PRKAR2A-AS1 PRKCA-AS1 PRKCQ-
AS1 PRKG1-AS1 PRKX-AS1 PRMT5-AS1 LOC101054525 PRR26 PANDAR PARTICL PROSER2-AS1 PART1
PCA3 PRNCR1 PCAT1 PCAT14 PCAT18 PCAT19 PCAT2 PCAT29 PCAT4 PCAT6 PCAT7 LOC440313
PROX1-AS1 PRR34-AS1 PRR7-AS1 PRRT3-AS1 PRRX2-AS1 PSMA3-AS1 PSMB8-AS1 PSMD5-AS1 PSMD6-
AS2 PSMG3-AS1 PRINS PSORS1C3 PTCHD1-AS PTENP1-AS PTGES2-AS1 PACERR PTOV1-AS1 PTOV1-
AS2 PTPRD-AS1 PTPRD-AS2 PTPRG-AS1 PTPRJ-AS1 LOC101060632 LOC101926984 LOC151760
LOC100507556 PVRL3-AS1 PVT1 PXN-AS1 PYCARD-AS1 RAB11B-AS1 RAB30-AS1 RAB4B-EGLN2
RAB6C-AS1 RAD21-AS1 RAD51-AS1 RAD51L3-RFFL RAET1E-AS1 RAI1-AS1 RALY-AS1 RAMP2-AS1
RAP2C-AS1 RAPGEF4-AS1 RARA-AS1 RASA2-IT1 RASA3-IT1 RASAL2-AS1 RASGRF2-AS1 RASSF1-AS1
RASSF8-AS1 RBAKDN RBFADN RBM12B-AS1 RBM26-AS1 RBM5-AS1 RBMS3-AS1 RBMS3-AS2 RBMS3-
AS3 RBPMS-AS1 RCAN3AS RDH10-AS1 RERG-AS1 RERG-IT1 RBSG2 RFPL1S RFPL3S RFX3-AS1 RGMB-
AS1 RGPD4-AS1 RMST RHOXF1-AS1 RHPN1-AS1 RPPH1 RMDN2-AS1 RMRP RN7SL1 RN7SL2 RNY1
RNY3 RNY4 RNY5 RNASEH1-AS1 RNASEH2B-AS1 RNASEK-C17orf49 RNF139-AS1 RNF144A-AS1
RNF157-AS1 RNF185-AS1 RNF216-IT1 RNF217-AS1 RNF219-AS1 ROPN1L-AS1 ROR1-AS1 RORA-AS1
RORA-AS2 RORB-AS1 RPARP-AS1 RPL34-AS1 RPS6KA2-AS1 RPS6KA2-IT1 RRS1-AS1 RSF1-IT1 RSF1-
IT2 RTCA-AS1 RTEL1-TNFRSF6B RUNDC3A-AS1 RUNX1-IT1 RUVBL1-AS1 SACS-AS1 SAMD12-AS1
SAMSN1-AS1 SAP30L-AS1 SAPCD1-AS1 SATB1-AS1 SATB2-AS1 SBF2-AS1 SCAANT1 SCAMP1-AS1
SCEL-AS1 SCOC-AS1 SDCBP2-AS1 SEC24B-AS1 SEMA3B-AS1 SEMA6A-AS1 SALRNA1 SALRNA2
SALRNA3 SENP3-EIF4A1 SEPSECS-AS1 SEPT4-AS1 SEPT5-GP1BB SEPT7-AS1 SERF2-C15ORF63
SERTAD4-AS1 SFTPD-AS1 SGMS1-AS1 SGOL1-AS1 SH3BP5-AS1 SH3PXD2A-AS1 SH3RF3-AS1 SHANK2-
AS1 SHANK2-AS2 SHANK2-AS3 SIAH2-AS1 SIDT1-AS1 SIRPG-AS1 SIX3-AS1 LOC101928202 SLC14A2-
AS1 SLC16A1-AS1 SLC16A12-AS1 SLC25A21-AS1 SLC25A25-AS1 SLC25A30-AS1 SLC25A5-AS1
SLC26A4-AS1 SLC2A1-AS1 SLC39A12-AS1 SLC6A1-AS1 SLC7A11-AS1 SLC8A1-AS1 SLC9A9-AS1
SLCO4A1-AS1 SLFNL1-AS1 SLIT1-AS1 SLIT2-IT1 SLMO2-ATP5E SLX1A-SULT1A3 SLX1B-SULT1A4
SMAD1-AS1 SMAD1-AS2 SMAD5-AS1 SMAD9-IT1 SCARNA1 SCARNA10 SCARNA11 SCARNA12
SCARNA13 SCARNA14 SCARNA15 SCARNA16 SCARNA17 SCARNA18 SCARNA2 SCARNA20
SCARNA21 SCARNA22 SCARNA23 SCARNA27 SCARNA3 SCARNA4 SCARNA5 SCARNA6 SCARNA7
SCARNA8 SCARNA9 SCARNA9L SMIM10L2A SMIM10L2B SNHG1 SNHG10 SNHG11 SNHG12 SNHG14
SNHG15 SNHG16 SNHG17 SNHG18 SNHG19 SNHG20 SNHG21 SNHG22 SNHG23 SNHG24 SNHG3 SNHG4
SNHG5 SNHG6 SNHG7 SNHG8 SNHG9 SMARCA5-AS1 SMC2-AS1 SMC5-AS1 SMG7-AS1 SMIM2-AS1
SMIM2-IT1 SMCR2 SMCR5 SMCR6 SENCR SNAI3-AS1 SNAP25-AS1 SNAP47-AS1 SNCA-AS1 SND1-IT1
SNRK-AS1 SOCS2-AS1 SORCS3-AS1 SOS1-IT1 SOX2-OT SOX21-AS1 SOX9-AS1 SP2-AS1 SPACA6P-AS
SPAG5-AS1 SPANXA2-OT1 SPATA13-AS1 SPATA17-AS1 SPATA3-AS1 SPATA8-AS1 SPECC1L-ADORA2A
SPACA6P SPATA41 SPATA42 SPG20-AS1 SPIN4-AS1 SFPQ SPRY4-IT1 SPTY2D1-AS1 SRD5A3-AS1
SRGAP2-AS1 SRGAP3-AS1 SRGAP3-AS2 SRGAP3-AS3 SRGAP3-AS4 SRP14-AS1 SRP54-AS1 SRRM2-AS1
SSBP3-AS1 SSSCA1-AS1 SSTR5-AS1 ST20-AS1 ST3GAL4-AS1 ST3GAL5-AS1 ST3GAL6-AS1 ST6GAL2-IT1
ST7-AS1 ST7-AS2 ST7-OT3 ST7-OT4 ST8SIA6-AS1 STAG3L5P-PVRIG2P-PILRB STAM-AS1 STARD13-AS
STARD13-IT1 STARD4-AS1 STARD7-AS1 STAU2-AS1 STEAP2-AS1 STEAP3-AS1 STK24-AS1 STK4-AS1
STPG2-AS1 STX16-NPEPL1 STX17-AS1 STX18-AS1 STX18-IT1 STXBP5-AS1 SUCLA2-AS1 SUCLG2-AS1
SVIL-AS1 SCHLAP1 SYNE1-AS1 SYNJ2-IT1 SYNPR-AS1 SYP-AS1 SYS1-DBNDD2 SZT2-AS1 TRG-AS1
TAB3-AS2 TAF1A-AS1 TAPT1-AS1 TAT-AS1 TUG1 TBC1D22A-AS1 TBL1XR1-AS1 TBX2-AS1 TBX5-AS1
TCEAL3-AS1 TCEB3-AS1 TCL6 TCERG1L-AS1 TARID TCF7L1-IT1 TUNAR TERC TEN1-CDK3 TESC-AS1
TDRG1 TEX41 TSL TTTY1 TTTY10 TTTY11 TTTY12 TTTY13 TTTY13B TTTY14 TTTY15 TTTY16
TTTY17A TTTY17B TTTY17C TTTY18 TTTY19 TTTY1B TTTY2 TTTY20 TTTY21 TTTY21B TTTY22
TTTY23 TTTY23B TTTY2B TTTY3 TTTY3B TTTY4 TTTY4B TTTY4C TTTY5 TTTY6 TTTY6B TTTY7
TTTY7B TTTY8 TTTY8B TTTY9A TTTY9B TET2-AS1 TEX26-AS1 TEX36-AS1 TFAP2A-AS1 TGFA-IT1
TGFB2-AS1 TGFB2-OT1 THAP7-AS1 THAP9-AS1 THOC7-AS1 THRB-AS1 THRB-IT1 THSD4-AS1 THSD4-
AS2 THUMPD3-AS1 THCAT126 TIPARP-AS1 TINCR TLR8-AS1 TLX1NB TM4SF1-AS1 TM4SF19-AS1
TM4SF19-TCTEX1D2 TMC3-AS1 TMCC1-AS1 TMEM108-AS1 TMEM147-AS1 TMEM161B-AS1 TMEM212-
AS1 TMEM212-IT1 TMEM220-AS1 TMEM246-AS1 TMEM254-AS1 TMEM256-PLSCR3 TMEM26-AS1
TMEM44-AS1 TMEM5-AS1 TMEM51-AS1 TMEM72-AS1 TMEM92-AS1 TMEM9B-AS1 TMLHE-AS1 TMPO-
AS1 TMPRSS4-AS1 TMX2-CTNND1 THRIL TNKS2-AS1 TNR-IT1 TNRC6C-AS1 TOB1-AS1 TOLLIP-AS1
TONSL-AS1 TOPORS-AS1 TP53TG1 TP73-AS1 TPRG1-AS1 TPRG1-AS2 TPT1-AS1 TRAF3IP2-AS1 TRAM2-
AS1 TRERNA1 TMEM75 TMEM78 TRAPPC12-AS1 TRHDE-AS1 TRIM31-AS1 TRIM36-IT1 TRIM52-AS1
TRPC7-AS1 TRPM2-AS TSC22D1-AS1 TSIX TSNAX-DISC1 TSPAN9-IT1 TSPEAR-AS1 TSPEAR-AS2
TSSC1-IT1 TTC21B-AS1 TTC28-AS1 TTC3-AS1 TTC39A-AS1 TTC39C-AS1 TTLL7-IT1 TTN-AS1 TUB-AS1
TP53COR1 TUSC7 TUSC8 TSG1 TXNDC12-AS1 UBA6-AS1 UBAC2-AS1 UBE2E1-AS1 UBE2F-SCLY
UBE2Q1-AS1 UBE2R2-AS1 UBL7-AS1 UBOX5-AS1 UBR5-AS1 UBXN10-AS1 UBXN7-AS1 UCHL1-AS1
UCKL1-AS1 UFL1-AS1 UGDH-AS1 UMODL1-AS1 UNC5B-AS1 RNR2 16S rRNA 16S rRNA RNA18S5
RNA28S5 RNA45S5 RNA5-8S5 RNA5S1 RNA5S10 RNA5S11 RNA5S12 RNA5S13 RNA5S14 RNA5S15
RNA5S16 RNA5S17 RNA5S2 RNA5S3 RNA5S4 RNA5S5 RNA5S6 RNA5S7 RNA5S8 RNA5S9 RNR1 12S
rRNA 12S rRNA RNU105B RNU105C RNU86 SNORD10 SNORD100 SNORD101 SNORD102 SNORD103A
SNORD103B SNORD104 SNORD105 SNORD105B SNORD107 SNORD108 SNORD109A SNORD109B
SNORD11 SNORD110 SNORD111 SNORD111B SNORD112 SNORD113-1 SNORD113-2 SNORD113-3
SNORD113-4 SNORD113-5 SNORD113-6 SNORD113-7 SNORD113-8 SNORD113-9 SNORD114-1
SNORD114-10 SNORD114-11 SNORD114-12 SNORD114-13 SNORD114-14 SNORD114-15 SNORD114-16
SNORD114-17 SNORD114-18 SNORD114-19 SNORD114-2 SNORD114-20 SNORD114-21 SNORD114-22
SNORD114-23 SNORD114-24 SNORD114-25 SNORD114-26 SNORD114-27 SNORD114-28 SNORD114-29
SNORD114-3 SNORD114-30 SNORD114-31 SNORD114-4 SNORD114-5 SNORD114-6 SNORD114-7
SNORD114-8 SNORD114-9 SNORD115-1 SNORD115-10 SNORD115-11 SNORD115-12 SNORD115-13
SNORD115-14 SNORD115-15 SNORD115-16 SNORD115-17 SNORD115-18 SNORD115-19 SNORD115-2
SNORD115-20 SNORD115-21 SNORD115-22 SNORD115-23 SNORD115-24 SNORD115-25 SNORD115-26
SNORD115-27 SNORD115-28 SNORD115-29 SNORD115-3 SNORD115-30 SNORD115-31 SNORD115-32
SNORD115-33 SNORD115-34 SNORD115-35 SNORD115-36 SNORD115-37 SNORD115-38 SNORD115-39
SNORD115-4 SNORD115-40 SNORD115-41 SNORD115-42 SNORD115-43 SNORD115-44 SNORD115-45
SNORD115-46 SNORD115-47 SNORD115-48 SNORD115-5 SNORD115-6 SNORD115-7 SNORD115-8
SNORD115-9 SNORD116-1 SNORD116-10 SNORD116-11 SNORD116-12 SNORD116-13 SNORD116-14
SNORD116-15 SNORD116-16 SNORD116-17 SNORD116-18 SNORD116-19 SNORD116-2 SNORD116-20
SNORD116-21 SNORD116-22 SNORD116-23 SNORD116-24 SNORD116-25 SNORD116-26 SNORD116-27
SNORD116-28 SNORD116-29 SNORD116-3 SNORD116-30 SNORD116-4 SNORD116-5 SNORD116-6
SNORD116-7 SNORD116-8 SNORD116-9 SNORD117 SNORD118 SNORD119 SNORD11B SNORD12
SNORD121A SNORD121B SNORD123 SNORD124 SNORD125 SNORD126 SNORD127 SNORD12B
SNORD12C SNORD13 SNORD14A SNORD14B SNORD14C SNORD14D SNORD14E SNORD15A
SNORD15B SNORD16 SNORD17 SNORD18A SNORD18B SNORD18C SNORD19 SNORD19B SNORD1A
SNORD1B SNORD1C SNORD2 SNORD20 SNORD21 SNORD22 SNORD23 SNORD24 SNORD25 SNORD26
SNORD27 SNORD28 SNORD29 SNORD30 SNORD31 SNORD32A SNORD32B SNORD33 SNORD34
SNORD35A SNORD35B SNORD36A SNORD36B SNORD36C SNORD37 SNORD38A SNORD38B SNORD3A
SNORD3B-1 SNORD3B-2 SNORD3C SNORD3D SNORD41 SNORD42A SNORD42B SNORD43 SNORD44
SNORD45A SNORD45B SNORD45C SNORD46 SNORD47 SNORD48 SNORD49A SNORD49B SNORD4A
SNORD4B SNORD5 SNORD50A SNORD50B SNORD51 SNORD52 SNORD53 SNORD54 SNORD55
SNORD56 SNORD56B SNORD57 SNORD58A SNORD58B SNORD58C SNORD59 SNORD59B SNORD6
SNORD60 SNORD61 SNORD62A SNORD62B SNORD63 SNORD64 SNORD65 SNORD66 SNORD67
SNORD68 SNORD69 SNORD7 SNORD70 SNORD71 SNORD72 SNORD73A SNORD74 SNORD75 SNORD76
SNORD77 SNORD78 SNORD79 SNORD8 SNORD80 SNORD81 SNORD82 SNORD83A SNORD83B
SNORD84 SNORD85 SNORD86 SNORD87 SNORD88A SNORD88B SNORD88C SNORD89 SNORD9
SNORD90 SNORD91A SNORD91B SNORD92 SNORD93 SNORD94 SNORD95 SNORD96A SNORD96B
SNORD97 SNORD98 SNORD99 SNORA1 SNORA10 SNORA11 SNORA11B SNORA11C SNORA11D
SNORA11E SNORA12 SNORA13 SNORA14A SNORA14B SNORA15 SNORA16A SNORA16B SNORA17
SNORA18 SNORA19 SNORA20 SNORA21 SNORA22 SNORA23 SNORA24 SNORA25 SNORA26 SNORA27
SNORA28 SNORA29 SNORA2A SNORA2B SNORA30 SNORA31 SNORA32 SNORA33 SNORA34 SNORA35
SNORA36A SNORA36B SNORA36C SNORA37 SNORA38 SNORA38B SNORA4 SNORA40 SNORA41
SNORA43 SNORA44 SNORA45A SNORA45B SNORA46 SNORA47 SNORA48 SNORA49 SNORA51
SNORA52 SNORA53 SNORA54 SNORA55 SNORA56 SNORA57 SNORA58 SNORA59A SNORA59B
SNORA5A SNORA5B SNORA5C SNORA6 SNORA60 SNORA61 SNORA62 SNORA63 SNORA64 SNORA65
SNORA66 SNORA67 SNORA68 SNORA69 SNORA70 SNORA70B SNORA70C SNORA70D SNORA70E
SNORA70F SNORA70G SNORA71A SNORA71B SNORA71C SNORA71D SNORA71E SNORA72
SNORA73A SNORA73B SNORA74A SNORA74B SNORA75 SNORA76A SNORA76C SNORA77 SNORA78
SNORA79 SNORA7A SNORA7B SNORA8 SNORA80A SNORA80B SNORA80E SNORA81 SNORA84
SNORA9 RN7SK RNU1-1 RNU1-13P RNU1-2 RNU1-27P RNU1-28P RNU1-3 RNU1-4 RNU11 RNU12 RNU2-1
RNU2-2P RNU4-1 RNU4-2 RNU4ATAC RNU5A-1 RNU5B-1 RNU5D-1 RNU5E-1 RNU5F-1 RNU6-1 RNU6-
10P RNU6-14P RNU6-15P RNU6-16P RNU6-19P RNU6-2 RNU6-21P RNU6-23P RNU6-26P RNU6-28P RNU6-
30P RNU6-31P RNU6-33P RNU6-34P RNU6-35P RNU6-36P RNU6-39P RNU6-42P RNU6-45P RNU6-46P
RNU6-48P RNU6-52P RNU6-53P RNU6-55P RNU6-56P RNU6-57P RNU6-58P RNU6-59P RNU6-63P RNU6-
64P RNU6-66P RNU6-67P RNU6-68P RNU6-69P RNU6-7 RNU6-71P RNU6-72P RNU6-75P RNU6-76P RNU6-
78P RNU6-79P RNU6-8 RNU6-81P RNU6-82P RNU6-83P RNU6-9 RNU6ATAC RNU7-1 RNVU1-1 RNVU1-14
RNVU1-15 RNVU1-17 RNVU1-18 RNVU1-19 RNVU1-20 RNVU1-3 RNVU1-4 RNVU1-6 RNVU1-7 RNVU1-8
SNAR-A1 SNAR-A10 SNAR-A11 SNAR-A12 SNAR-A13 SNAR-A14 SNAR-A2 SNAR-A3 SNAR-A4 SNAR-
A5 SNAR-A6 SNAR-A7 SNAR-A8 SNAR-A9 SNAR-B1 SNAR-B2 SNAR-C1 SNAR-C2 SNAR-C3 SNAR-C4
SNAR-C5 SNAR-D SNAR-E SNAR-F SNAR-G1 SNAR-G2 SNAR-H SNAR-I NMTRQ-TTG14-1 NMTRQ-
TTG3-1 NMTRQ-TTG5-1 NMTRL-TAA1-1 NMTRL-TAA4-1 NMTRS-TGA1-1 TRNAA-AGC TRNAA-CGC
TRNAA-UGC TRR TRNAR-ACG TRNAR-CCG TRNAR-CCU TRNAR-UCG TRNAR-UCU TRNAN-GUU
TRNAD-GUC TRNAC-GCA TRNAE-CUC TRNAE-UUC TRNAQ-CUG TRNAQ-UUG TRNAG-CCC TRNAG-
GCC TRNAG-UCC TRNAH-GUG TRNAI-AAU TRNAI-GAU TRNAI-UAU TRNAL-AAG TRNAL-CAA
TRNAL-CAG TRNAL-UAA TRNAL-UAG TRNAK-CUU TRNAK-UUU TRNAM-CAU TRNASTOP-UUA
TRNASTOP-UCA TRNAF-GAA TRNAP-AGG TRNAP-CGG TRNAP-UGG TRNAS-AGA TRNAS-CGA
TRNAS-GCU TRNAS-UGA TRSUP-CTA1-1 TRSUP-TTA1-1 TRSUP-TTA2-1 TRNAT-AGU TRNAT-CGU
TRNAT-UGU TRNAW-CCA TRNAY-AUA TRNAY-GUA TRNAV-AAC TRNAV-CAC TRNAV-UAC TRA-
AGC10-1 TRA-AGC1-1 TRA-AGC11-1 TRA-AGC12-1 TRA-AGC12-2 TRA-AGC12-3 TRA-AGC13-1 TRA-
AGC13-2 TRA-AGC14-1 TRA-AGC15-1 TRA-AGC16-1 TRA-AGC17-1 TRA-AGC18-1 TRA-AGC18-2 TRA-
AGC19-1 TRA-AGC20-1 TRA-AGC2-1 TRA-AGC21-1 TRA-AGC2-2 TRA-AGC22-1 TRA-AGC3-1 TRA-
AGC4-1 TRA-AGC5-1 TRA-AGC6-1 TRA-AGC7-1 TRA-AGC8-1 TRA-AGC8-2 TRA-AGC9-1 TRA-AGC9-2
TRA-CGC1-1 TRA-CGC2-1 TRA-CGC3-1 TRA-CGC4-1 TRA-CGC5-1 TRA-TGC1-1 TRA-TGC2-1 TRA-
TGC3-1 TRA-TGC3-2 TRA-TGC4-1 TRA-TGC5-1 TRA-TGC6-1 TRA-TGC7-1 TRA-TGC8-1 TRR-ACG1-1
TRR-ACG1-2 TRR-ACG1-3 TRR-ACG2-1 TRR-ACG2-2 TRR-ACG2-3 TRR-ACG2-4 TRR-CCG1-1 TRR-
CCG1-2 TRR-CCG1-3 TRR-CCG2-1 TRR-CCT1-1 TRR-CCT2-1 TRR-CCT3-1 TRR-CCT4-1 TRR-CCT5-1 TRR-
TCG1-1 TRR-TCG2-1 TRR-TCG3-1 TRR-TCG4-1 TRR-TCG5-1 TRR-TCG6-1 TRR-TCT1-1 TRR-TCT2-1 TRR-
TCT3-1 TRR-TCT3-2 TRR-TCT4-1 TRR-TCT5-1 TRN-ATT1-1 TRN-ATT1-2 TRN-GTT10-1 TRN-GTT1-1
TRN-GTT11-1 TRN-GTT11-2 TRN-GTT12-1 TRN-GTT13-1 TRN-GTT14-1 TRN-GTT15-1 TRN-GTT16-1
TRN-GTT16-2 TRN-GTT16-3 TRN-GTT16-4 TRN-GTT16-5 TRN-GTT17-1 TRN-GTT18-1 TRN-GTT19-1
TRN-GTT20-1 TRN-GTT2-1 TRN-GTT21-1 TRN-GTT2-2 TRN-GTT2-3 TRN-GTT2-4 TRN-GTT2-5 TRN-
GTT2-6 TRN-GTT3-1 TRN-GTT3-2 TRN-GTT4-1 TRN-GTT5-1 TRN-GTT6-1 TRN-GTT7-1 TRN-GTT8-1
TRN-GTT9-1 TRN-GTT9-2 TRD-GTC1-1 TRD-GTC2-1 TRD-GTC2-10 TRD-GTC2-11 TRD-GTC2-2 TRD-
GTC2-3 TRD-GTC2-4 TRD-GTC2-5 TRD-GTC2-6 TRD-GTC2-7 TRD-GTC2-8 TRD-GTC2-9 TRD-GTC3-1
TRD-GTC4-1 TRD-GTC5-1 TRD-GTC6-1 TRD-GTC7-1 TRD-GTC8-1 TRD-GTC9-1 TRC-GCA10-1 TRC-
GCA1-1 TRC-GCA11-1 TRC-GCA12-1 TRC-GCA13-1 TRC-GCA14-1 TRC-GCA15-1 TRC-GCA16-1 TRC-
GCA17-1 TRC-GCA18-1 TRC-GCA19-1 TRC-GCA20-1 TRC-GCA2-1 TRC-GCA21-1 TRC-GCA2-2 TRC-
GCA22-1 TRC-GCA2-3 TRC-GCA23-1 TRC-GCA2-4 TRC-GCA24-1 TRC-GCA3-1 TRC-GCA4-1 TRC-GCA5-1
TRC-GCA6-1 TRC-GCA7-1 TRC-GCA8-1 TRC-GCA9-1 TRC-GCA9-2 TRC-GCA9-3 TRC-GCA9-4 TRQ-
CTG10-1 TRQ-CTG1-1 TRQ-CTG1-2 TRQ-CTG12-1 TRQ-CTG1-3 TRQ-CTG1-4 TRQ-CTG14-1 TRQ-CTG1-5
TRQ-CTG15-1 TRQ-CTG17-1 TRQ-CTG18-1 TRQ-CTG2-1 TRQ-CTG3-1 TRQ-CTG3-2 TRQ-CTG4-1 TRQ-
CTG4-2 TRQ-CTG5-1 TRQ-CTG6-1 TRQ-CTG7-1 TRQ-CTG8-1 TRQ-CTG8-2 TRQ-TTG10-1 TRQ-TTG1-1
TRQ-TTG2-1 TRQ-TTG3-1 TRQ-TTG3-2 TRQ-TTG3-3 TRQ-TTG4-1 TRQ-TTG6-1 TRE-CTC1-1 TRE-CTC1-2
TRE-CTC1-3 TRE-CTC1-4 TRE-CTC1-5 TRE-CTC1-6 TRE-CTC1-7 TRE-CTC17-1 TRE-CTC2-1 TRE-CTC3-1
TRE-CTC5-1 TRE-CTC6-1 TRE-CTC8-1 TRE-TTC1-1 TRE-TTC11-1 TRE-TTC1-2 TRE-TTC12-1 TRE-TTC13-
1 TRE-TTC16-1 TRE-TTC2-1 TRE-TTC2-2 TRE-TTC3-1 TRE-TTC4-1 TRE-TTC4-2 TRE-TTC5-1 TRE-TTC8-1
TRG-CCC1-1 TRG-CCC1-2 TRG-CCC2-1 TRG-CCC2-2 TRG-CCC3-1 TRG-CCC5-1 TRG-CCC8-1 TRG-GCC1-
1 TRG-GCC1-2 TRG-GCC1-3 TRG-GCC1-4 TRG-GCC1-5 TRG-GCC2-1 TRG-GCC2-2 TRG-GCC2-3 TRG-
GCC2-4 TRG-GCC2-5 TRG-GCC2-6 TRG-GCC3-1 TRG-GCC4-1 TRG-GCC5-1 TRG-GCC6-1 TRG-TCC1-1
TRG-TCC2-1 TRG-TCC2-2 TRG-TCC2-3 TRG-TCC2-4 TRG-TCC2-5 TRG-TCC2-6 TRG-TCC3-1 TRG-TCC4-1
TRH-GTG1-1 TRH-GTG1-2 TRH-GTG1-3 TRH-GTG1-4 TRH-GTG1-5 TRH-GTG1-6 TRH-GTG1-7 TRH-
GTG1-8 TRH-GTG1-9 TRH-GTG2-1 TRH-GTG3-1 TRI-AAT1-1 TRI-AAT2-1 TRI-AAT3-1 TRI-AAT4-1 TRI-
AAT5-1 TRI-AAT5-2 TRI-AAT5-3 TRI-AAT5-4 TRI-AAT5-5 TRI-AAT6-1 TRI-AAT7-1 TRI-AAT7-2 TRI-
AAT8-1 TRI-AAT9-1 TRI-GAT1-1 TRI-GAT1-2 TRI-GAT1-3 TRI-TAT1-1 TRI-TAT2-1 TRI-TAT2-2 TRI-
TAT2-3 TRI-TAT3-1 TRX-CAT1-1 TRX-CAT1-2 TRX-CAT1-3 TRX-CAT1-4 TRX-CAT1-5 TRX-CAT1-6
TRX-CAT1-7 TRX-CAT1-8 TRX-CAT2-1 TRX-CAT3-1 TRL-AAG1-1 TRL-AAG1-2 TRL-AAG1-3 TRL-AAG2-
1 TRL-AAG2-2 TRL-AAG2-3 TRL-AAG2-4 TRL-AAG3-1 TRL-AAG4-1 TRL-AAG5-1 TRL-AAG6-1 TRL-
AAG8-1 TRL-CAA1-1 TRL-CAA1-2 TRL-CAA2-1 TRL-CAA3-1 TRL-CAA4-1 TRL-CAA5-1 TRL-CAA6-1
TRL-CAG1-1 TRL-CAG1-2 TRL-CAG1-3 TRL-CAG1-4 TRL-CAG1-5 TRL-CAG1-6 TRL-CAG1-7 TRL-CAG2-
1 TRL-CAG2-2 TRL-CAG3-1 TRL-TAA1-1 TRL-TAA2-1 TRL-TAA3-1 TRL-TAA4-1 TRL-TAA5-1 TRL-
TAG1-1 TRL-TAG2-1 TRL-TAG3-1 TRK-CTT10-1 TRK-CTT1-1 TRK-CTT11-1 TRK-CTT1-2 TRK-CTT15-1
TRK-CTT2-1 TRK-CTT2-2 TRK-CTT2-3 TRK-CTT2-4 TRK-CTT2-5 TRK-CTT3-1 TRK-CTT4-1 TRK-CTT5-1
TRK-CTT6-1 TRK-CTT8-1 TRK-CTT9-1 TRK-TTT1-1 TRK-TTT11-1 TRK-TTT12-1 TRK-TTT16-1 TRK-
TTT2-1 TRK-TTT3-1 TRK-TTT3-2 TRK-TTT3-3 TRK-TTT3-4 TRK-TTT3-5 TRK-TTT4-1 TRK-TTT5-1 TRK-
TTT6-1 TRK-TTT7-1 TRK-TTT8-1 TRK-TTT9-1 TRM-CAT1-1 TRM-CAT2-1 TRM-CAT3-1 TRM-CAT3-2
TRM-CAT4-1 TRM-CAT4-2 TRM-CAT4-3 TRM-CAT5-1 TRM-CAT6-1 TRM-CAT7-1 TRF-GAA1-1 TRF-
GAA1-2 TRF-GAA1-3 TRF-GAA1-4 TRF-GAA1-5 TRF-GAA1-6 TRF-GAA2-1 TRF-GAA3-1 TRF-GAA4-1
TRF-GAA5-1 TRF-GAA6-1 TRF-GAA7-1 TRP-AGG1-1 TRP-AGG2-1 TRP-AGG2-2 TRP-AGG2-3 TRP-AGG2-
4 TRP-AGG2-5 TRP-AGG2-6 TRP-AGG2-7 TRP-AGG2-8 TRP-AGG3-1 TRP-CGG1-1 TRP-CGG1-2 TRP-
CGG1-3 TRP-CGG2-1 TRP-TGG1-1 TRP-TGG2-1 TRP-TGG3-1 TRP-TGG3-2 TRP-TGG3-3 TRP-TGG3-4 TRP-
TGG3-5 TRU-TCA1-1 TRU-TCA2-1 TRU-TCA3-1 TRS-AGA1-1 TRS-AGA2-1 TRS-AGA2-2 TRS-AGA2-3
TRS-AGA2-4 TRS-AGA2-5 TRS-AGA2-6 TRS-AGA3-1 TRS-AGA4-1 TRS-AGA5-1 TRS-AGA6-1 TRS-CGA1-
1 TRS-CGA2-1 TRS-CGA3-1 TRS-CGA4-1 TRS-GCT1-1 TRS-GCT2-1 TRS-GCT3-1 TRS-GCT4-1 TRS-GCT4-
2 TRS-GCT4-3 TRS-GCT5-1 TRS-GCT6-1 TRS-TGA1-1 TRS-TGA2-1 TRS-TGA3-1 TRS-TGA4-1 TRT-AGT1-
1 TRT-AGT1-2 TRT-AGT1-3 TRT-AGT2-1 TRT-AGT2-2 TRT-AGT3-1 TRT-AGT4-1 TRT-AGT5-1 TRT-
AGT6-1 TRT-AGT7-1 TRT-CGT1-1 TRT-CGT2-1 TRT-CGT3-1 TRT-CGT4-1 TRT-CGT5-1 TRT-CGT6-1 TRT-
TGT1-1 TRT-TGT2-1 TRT-TGT3-1 TRT-TGT4-1 TRT-TGT5-1 TRT-TGT6-1 TRW-CCA1-1 TRW-CCA2-1
TRW-CCA3-1 TRW-CCA3-2 TRW-CCA3-3 TRW-CCA4-1 TRW-CCA5-1 TRW-CCA6-1 TRW-CCA7-1 TRY-
ATA1-1 TRY-GTA10-1 TRY-GTA1-1 TRY-GTA2-1 TRY-GTA3-1 TRY-GTA4-1 TRY-GTA5-1 TRY-GTA5-2
TRY-GTA5-3 TRY-GTA5-4 TRY-GTA5-5 TRY-GTA6-1 TRY-GTA7-1 TRY-GTA8-1 TRY-GTA9-1 TRV-
AAC1-1 TRV-AAC1-2 TRV-AAC1-3 TRV-AAC1-4 TRV-AAC1-5 TRV-AAC2-1 TRV-AAC3-1 TRV-AAC4-1
TRV-AAC5-1 TRV-AAC6-1 TRV-AAC7-1 TRV-CAC10-1 TRV-CAC1-1 TRV-CAC11-1 TRV-CAC1-2 TRV-
CAC12-1 TRV-CAC1-3 TRV-CAC1-4 TRV-CAC1-5 TRV-CAC1-6 TRV-CAC2-1 TRV-CAC3-1 TRV-CAC4-1
TRV-CAC5-1 TRV-CAC6-1 TRV-CAC7-1 TRV-CAC8-1 TRV-CAC9-1 TRV-TAC1-1 TRV-TAC1-2 TRV-
TAC2-1 TRV-TAC3-1 TRV-TAC4-1 TRNC TRNA TRND TRNE TRNF TRNG TRNH TRNI TRNK TRNL1
TRNL2 TRNM TRNN TRNP TRNQ TRNR TRNS1 TRNS2 TRNT TRNV TRNW TRNY trnT trnE trnL trnS trnH
trnR trnG trnK trnS trnD trnY trnC trnL trnF trnP trnV trnN trnW trnA trnQ trnM trnI trnF trnV trnL trnS trnK trnG
trnT trnI trnW trnR trnH trnE trnC trnY trnM trnS trnQ trnL trnD trnP trnA TRNAG1

TABLE 13
Cell surface markers in maturing erythroid cells

| | Markers | | | |
|---|---|---|---|---|
| | | | | Alpha4 integrin- |
| | GPA- | Alpha4 integrin- | Band3- | positive and |
| Stage | positive | positive | positive | Band3-positive |
| M0 | 83.9% | 98.0% | 54.6% | 52.9% |
| M3 | 99.0% | 91.4% | 97.8% | 89.6% |
| M5 | 99.5% | 84.2% | 100% | 84.2% |

TABLE 14
Co-expression of EGFP and mCherry

| | Percent cells positive for: | | |
|---|---|---|---|
| Stage | EGFP only | mCherry only | EGFP and mCherry |
| M6 | 90.4% | 89.05% | 86.55% |
| M11 | 77.75% | 75.90% | 66.05% |
| M13 | 86.00% | 80.30% | 75.40% |
| M18 | 94.15% | 90.80% | 86.40% |

TABLE 15
Co-expression of 4-1BBL and Avelumab

| | Percent cells positive for: | | |
|---|---|---|---|
| | | | 4-1BBL and |
| Sample | 4-1BBL | Avelumab | Avelumab |
| Negative control | 0.99% | 0.24% | 0.035% |
| Erythroid cells + m4- | 92.5% | 0.58% | 0.39% |
| 1BBL only | | | |
| Erythroid cells + | 1.4% | 75.0% | 0.59% |
| Avelumab only | | | |
| Erythroid cells + m4- | 61.4% | 70.9% | 58.5% |
| 1BBL and avelumab | | | |

TABLE 16
Dose expression results

| Amount mRNA | Percent of cells | Number of copies of 4-1BBL |
|---|---|---|
| added (mg) | expressing 4-1BBL | per cell |
| 0.6 | 87.5% | 1,015,250 |
| 0.4 | 90.6% | 874,017 |
| 0.2 | 92.0% | 609,145 |
| 0.1 | 91.75% | 274,766 |
| 0.05 | 87.7% | 100,500 |
| 0.025 | 74.0% | 42,902 |
| 0 | 1.25% | NA |

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed is:

1. A method of making an erythroid cell comprising an mRNA encoding an exogenous protein, comprising:
a) providing an erythroid cell in maturation phase, and
b) contacting the erythroid cell with an mRNA encoding the exogenous protein, under conditions that allow uptake of the mRNA by the erythroid cell,
thereby making an erythroid cell comprising an mRNA encoding an exogenous protein.

2. The method of claim 1, wherein the erythroid cell takes up the mRNA encoding the exogenous protein.

3. The method of claim 1, comprising providing a population of erythroid cells in maturation phase and contacting a plurality of cells of the population of erythroid cells with the mRNA encoding the exogenous protein.

4. The method of claim 3, wherein the plurality of cells of the population of erythroid cells each takes up the mRNA encoding the exogenous protein.

5. The method of any of claims 1-4, wherein after uptake of the mRNA encoding the exogenous protein, the cell or the plurality of cells express the exogenous protein.

6. The method of claim 5, wherein the cell or the plurality of cells comprise the exogenous protein.

7. The method of any of claims 3-6, wherein the population of erythroid cells in maturation phase is a population of cells expanded in a maturation medium for 3-7 days, e.g., 4-5 or 4-6 days.

8. A method of manufacturing a population of reticulocytes that express an exogenous protein, the method comprising
(e) providing a population of erythroid precursor cells (e.g., CD34+ cells);
(f) culturing the population of erythroid precursor cells under differentiating conditions to provide a population of differentiating erythroid cells;
(g) contacting a plurality of cells of the population of differentiating erythroid cells with an mRNA encoding the exogenous protein, under conditions that allow uptake of the mRNA by the plurality of cells of the population of differentiating erythroid cells; and
(h) further culturing the plurality of cells of the population of differentiating erythroid cells to provide a population of reticulocytes,
thereby manufacturing a population of reticulocytes that express the exogenous protein.

9. The method of claim 8, wherein the further culturing comprises fewer than 3, 2, or 1 population doubling.

10. The method of any of claims 3-9, wherein the population of erythroid cells or the population of differentiating erythroid cells is a population of erythroid cells comprising one or more (e.g., 2, 3, 4, 5, 6, 7, 8, 9, 10, 11, 12, 13, 14, 15, 16, 17, or more) of the following properties:
i.a) 2-40%, 3-33%, 5-30%, 10-25%, or 15-20% of the cells in the population are enucleated;
i.b) greater than 0%, 0.1%, 0.2%, or 0.5%, but less than 2%, 3%, 4%, or 5% of the cells in the population are enucleated;
i.c) greater than 0%, 0.1%, 0.2%, or 0.5%, but less than 6%, 10%, 15%, 20%, or 25% of the cells in the population are enucleated;
i.d) greater than 0%, 0.1%, 0.2%, or 0.5%, but less than 30%, 35%, 40%, 45%, or 50% of the cells in the population are enucleated;
i.e) no more than 1%, 2%, 3%, 5%, 10%, 15%, or 20% of the cells in the population are enucleated;
i.f) no more than 25%, 30%, 35%, 40%, 45%, or 50% of the cells in the population are enucleated;
i.g) the population of cells has reached 6-70%, 10-60%, 20-50%, or 30-40% of maximal enucleation;
i.h) the population of cells has reached no more than 1%, 2%, 3%, 5%, 10%, 15%, or 20% of maximal enucleation;
i.i) the population of cells has reached no more than 25%, 30%, 35%, 40%, 45%, 50%, or 60% of maximal enucleation;
ii.a) the population of cells is fewer than 3, 2, or 1 population doubling from a plateau in cell division;
ii.b) the population of cells is capable of fewer than 3, 2, or 1 population doubling;
ii.c) the population will increase by no more than 1.5, 2, or 3 fold before the population reaches an enucleation level of at least 70% of cells in the population;
iii.a) at least 80%, 85%, 90%, 95%, or 99% of the cells in the population are normoblasts (e.g., polychromatic or orthochromatic normoblasts);
iii.b) at least 50%, 60%, 70%, 75%, or 79% of the cells in the population are normoblasts (e.g., polychromatic or orthochromatic normoblasts);
iii.c) 30-90%, 40-90%, 50-90%, 60-90%, or 70-90% of the cells in the population are normoblasts (e.g., polychromatic or orthochromatic normoblasts);
iii.d) at least 80%, 85%, 90%, 95%, or 99% of the cells in the population exhibit the morphology of a normoblast (e.g., a polychromatic or orthochromatic normoblast);
iii.e) at least 50%, 60%, 70%, 75%, or 79% of the cells in the population exhibit the morphology of a normoblast (e.g., a polychromatic or orthochromatic normoblast); or
iii.f) 30-90%, 40-90%, 50-90%, 60-90%, or 70-90% of the cells in the population exhibit the morphology of a normoblast (e.g., a polychromatic or orthochromatic normoblast).

11. The method of claim 10, wherein the population of erythroid cells or the population of differentiating erythroid cells is a population of erythroid cells comprising a property from i and a property from ii.

12. The method of claim 10, wherein the population of erythroid cells or the population of differentiating erythroid cells is a population of erythroid cells comprising a property from i and a property from iii.

13. The method of claim 10, wherein the population of erythroid cells or the population of differentiating erythroid cells is a population of erythroid cells comprising a property from ii and a property from iii.

14. The method of claim 10, wherein the population of erythroid cells or the population of differentiating erythroid cells is a population of erythroid cells comprising a property from i, a property from ii, and a property from iii.

15. The method of any of claims 3-14, wherein the population of erythroid cells or the population of differentiating erythroid cells is a population of erythroid cells comprising the following property: 84-99%, 85-95%, or about 90% of the cells in the population are GPA-positive, e.g., as measured by a flow cytometry assay, e.g., a flow cytometry assay of Example 10.

16. The method of any of claims 3-14, wherein the population of erythroid cells or the population of differentiating erythroid cells is a population of erythroid cells comprising the following property: at least 84%, 85%, 90%, 95%, or 99% of the cells in the population are GPA-positive, e.g., as measured by a flow cytometry assay, e.g., a flow cytometry assay of Example 10.

17. The method of any of claims 3-14, wherein the population of erythroid cells or the population of differentiating erythroid cells is a population of erythroid cells comprising the following property: 54-99%, 55-98%, 60-95%, 65-90%, 70-85%, or 75-80% of the cells in the population are band3-positive, e.g., as measured by a flow cytometry assay, e.g., a flow cytometry assay of Example 10.

18. The method of any of claims 3-14, wherein the population of erythroid cells or the population of differentiating erythroid cells is a population of erythroid cells comprising the following property: at least 54%, 55%, 60%, 65%, 70%, 75%, 80%, 85%, 90%, 95%, 98%, or 99% of the cells in the population are band3-positive, e.g., as measured by a flow cytometry assay, e.g., a flow cytometry assay of Example 10.

19. The method of any of claims 3-14, wherein the population of erythroid cells or the population of differentiating erythroid cells is a population of erythroid cells comprising the following property: 96-100%, 97-99%, or about 98% of the cells in the population are alpha4 integrin-positive, e.g., as measured by a flow cytometry assay, e.g., a flow cytometry assay of Example 10.

20. The method of any of claims 3-14, wherein the population of erythroid cells or the population of differentiating erythroid cells is a population of erythroid cells comprising the following property: at least 95%, 96%, 97%, 98%, or 99% of the cells in the population are alpha4 integrin-positive, e.g., as measured by a flow cytometry assay, e.g., a flow cytometry assay of Example 10.

21. The method of any of claims 3-20, wherein prior to or after contacting the plurality of cells with the mRNA encoding the exogenous protein, the plurality of cells are separated from the population of erythroid cells or the population of differentiating erythroid cells, e.g., the plurality of cells are separated from the population based on enucleation status (e.g., the plurality of cells are nucleated cells and the rest of the population are enucleated cells).

22. The method of any of claims 3-20, comprising prior to or after contacting the plurality of cells with the mRNA encoding the exogenous protein, synchronizing the population of erythroid cells or the population of differentiating erythroid cells, e.g., by arresting the growth, development, hemoglobin synthesis, or the process of enucleation of the population, e.g., by incubating the population with an inhibitor of enucleation (e.g., an inhibitor of histone deacetylase (HDAC), an inhibitor of mitogen-activated protein kinase (MAPK), an inhibitor of cyclin-dependent kinase (CDK), or a proteasome inhibitor).

23. The method of claim 22, wherein arresting occurs prior to enucleation of more than 1, 2, 3, 4, 5, 6, 7, 8, 9 or 10% of the cells in the population.

24. A method of manufacturing a population of reticulocytes that express an exogenous protein, the method comprising:
(i) providing a population of erythroid precursor cells (e.g., CD34+ cells);
(j) culturing the population of erythroid precursor cells under differentiating conditions to provide a population of differentiating erythroid cells;
(k) contacting the differentiating erythroid cells with an mRNA encoding the exogenous protein, under conditions that allow uptake of the mRNA by the differentiating erythroid cells, wherein the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated); and
(l) further culturing the differentiating erythroid cells to provide a population of reticulocytes,
thereby manufacturing a population of reticulocytes that express the exogenous protein.

25. The method of claim 24, wherein the further culturing comprises fewer than 3, 2, or 1 population doubling.

26. The method of claim 24 or 25, wherein the contacting is performed when at least 50% (at least 60%, 70%, 75%, 80%, 90%, or 95%) of the differentiating erythroid cells exhibit the morphology of a normoblast (e.g., a polychromatic or orthochromatic normoblast).

27. A method of manufacturing a population of reticulocytes that express an exogenous protein, comprising (a) providing a population of erythroid precursor cells, (b) culturing the population of erythroid precursor cells under differentiating conditions to provide a population of differentiating erythroid cells, (c) contacting the differentiating erythroid cells with an mRNA encoding the exogenous protein, wherein the improvement comprises: the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated).

28. The method of claim 27, wherein the contacting is performed when the population of differentiating erythroid cells has fewer than 3, 2, or 1 population doubling before a plateau in cell division.

29. The method of claim 27 or 28, wherein the contacting is performed when at least 50% (at least 60%, 70%, 75%, 80%, 90%, or 95%) of the differentiating erythroid cells exhibit the morphology of a normoblast (e.g., a polychromatic or orthochromatic normoblast).

30. An erythroid cell, e.g., an enucleated erythroid cell, comprising:
an exogenous mRNA comprising a coding region operatively linked to a heterologous untranslated region (UTR), wherein the heterologous UTR comprises a regulatory element.

31. An erythroid cell, e.g., an enucleated erythroid cell, comprising an exogenous mRNA that comprises one or more chemically modified nucleotides of Table 1, one or more chemical backbone modifications of Table 2, one or more chemically modified caps of Table 3, or any combination thereof.

32. A method of producing an erythroid cell, e.g., enucleated erythroid cell, comprising:
a) contacting an erythroid cell, e.g., a nucleated erythroid cell, with an exogenous mRNA comprising a coding region operatively linked to a heterologous UTR comprising a regulatory element, (e.g., isolated RNA or in vitro transcribed RNA), and
b) maintaining the contacted erythroid cell under conditions suitable for uptake of the exogenous mRNA,
thereby producing the erythroid cell, e.g., an enucleated erythroid cell.

33. A method of producing an erythroid cell, e.g., enucleated erythroid cell, comprising:
a) contacting an erythroid cell, e.g., a nucleated erythroid cell, with an exogenous mRNA comprising one or more chemically modified nucleotides of Table 1, one or more chemical backbone modifications of Table 2, one or more chemically modified caps of Table 3, or any combination thereof; and
b) maintaining the contacted erythroid cell under conditions suitable for uptake of the exogenous mRNA,
thereby producing the erythroid cell, e.g., an enucleated erythroid cell.

34. A method of producing an exogenous protein in an enucleated erythroid cell:
a) providing an erythroid cell, e.g., a nucleated erythroid cell, comprising an exogenous mRNA comprising a coding region operatively linked to a heterologous UTR comprising a regulatory element, (e.g., isolated RNA or in vitro transcribed RNA), and
b) culturing the erythroid cell under conditions suitable for production of the exogenous protein,
thereby producing the exogenous protein.

35. A method of producing an exogenous protein in an enucleated erythroid cell:
a) providing an erythroid cell, e.g., a nucleated erythroid cell, comprising one or more chemically modified nucleotides of Table 1, one or more chemical backbone modifications of Table 2, one or more chemically modified caps of Table 3, or any combination thereof, and
b) culturing the erythroid cell under conditions suitable for production of the exogenous protein,
thereby producing the exogenous protein.

36. A method of providing a subject with an exogenous protein, providing a subject with an enucleated erythroid cell which can produce an exogenous protein, or treating a subject, comprising administering to the subject:
an erythroid cell, e.g., a nucleated erythroid cell, comprising an exogenous mRNA comprising a coding region operatively linked to a heterologous UTR comprising a regulatory element, (e.g., isolated RNA or in vitro transcribed RNA),
thereby providing a subject with an exogenous protein, providing a subject with an enucleated erythroid cell which can produce an exogenous protein, or treating a subject.

37. A method of providing a subject with an exogenous protein, providing a subject with an enucleated erythroid cell which can produce an exogenous protein, or treating a subject, comprising administering to the subject:
an erythroid cell, e.g., a nucleated erythroid cell, comprising an exogenous mRNA comprising one or more chemically modified nucleotides of Table 1, one or more chemical backbone modifications of Table 2, one or more chemically modified caps of Table 3, or any combination thereof,
thereby providing a subject with an exogenous protein, providing a subject with an enucleated erythroid cell which can produce an exogenous protein, or treating a subject.

38. A method of evaluating an erythroid cell, e.g., enucleated erythroid cell (or a batch of such cells) comprising:
a) providing an erythroid cell, e.g., a nucleated erythroid cell, comprising an exogenous mRNA comprising a coding region operatively linked to a heterologous UTR comprising a regulatory element (or a batch of such cells), and
b) evaluating the erythroid cell, e.g., the nucleated erythroid cell (or batch of such cells) for a preselected parameter,
thereby evaluating the erythroid cell, e.g., enucleated erythroid cell (or a batch of such cells).

39. A method of evaluating an erythroid cell, e.g., enucleated erythroid cell (or a batch of such cells) comprising:
a) providing an erythroid cell, e.g., a nucleated erythroid cell, comprising one or more chemically modified nucleotides of Table 1, one or more chemical backbone modifications of Table 2, one or more chemically modified caps of Table 3, or any combination thereof, and
b) evaluating the erythroid cell, e.g., the nucleated erythroid cell (or batch of such cells) for a preselected parameter,
thereby evaluating the erythroid cell, e.g., enucleated erythroid cell (or a batch of such cells).

40. The method of claim 30, wherein at least 50%, 60%, 70%, 80%, 85%, 90%, or 95% of the cells in the population comprise the exogenous protein, e.g., 5 days after contacting with the mRNA.

41. The method of claim 30, wherein the cells in the population comprise at least 1,000, 2,000, 5,000, 10,000, 20,000, 50,000, or 100,000 copies of the exogenous protein, e.g., 5 days after the contacting with the mRNA.

42. The method of claim 30, wherein the cells comprise at least 1,000, 2,000, 5,000, 10,000, 20,000, 50,000, or 100,000 copies of the exogenous protein for at least 5, 6, 7, 8, 9, 10, 11, 12, 13, 14, or 15 days after contacting with the mRNA.

43. A method of making an erythroid cell comprising an mRNA that encodes an exogenous protein, comprising:
providing a reaction mixture comprising an erythroid cell and an mRNA encoding the exogenous protein, under conditions which inhibit degradation of mRNA, e.g., by inclusion in the reaction mixture a ribonuclease inhibitor, and
maintaining the reaction mixture under conditions that allow uptake of the mRNA by the erythroid cell,
thereby making an erythroid cell comprising an mRNA encoding an exogenous protein.

44. The method of claim 43, comprising providing a population of erythroid cells and contacting the population with the mRNA encoding the exogenous protein.

45. The method of claim 43 or 44, wherein a plurality of erythroid cells of the population each takes up an mRNA encoding the exogenous protein.

46. The method of any of claims 43-45, wherein the cell or plurality of cells express the exogenous protein.

47. The method of any of claims 43-46, wherein the cell or plurality of cells comprise the exogenous protein.

48. The method of any of claims 43-47, which further comprises electroporating the cell or population of cells.

49. The method of any of claims 43-48, which further comprises contacting a population of erythroid cells with a ribonuclease inhibitor.

50. The method of any of claims 43-49, which comprises contacting the population of cells with the ribonuclease inhibitor before, during, or after contacting the cells with the mRNA.

51. The method of any of claims 43-50, which comprises contacting the cells with the ribonuclease inhibitor at day 4, 5, or 6 of maturation phase.

52. The method of any of claims 43-51, wherein the cell is in maturation phase.

53. The method of any of claims 43-52, which comprises contacting the cells with the ribonuclease inhibitor at a time when the cells comprise one or more (e.g., 2, 3, 4, 5, 6, 7, 8, 9, 10, 11, 12, 13, 14, 15, 16, 17, or more) of the following properties:
i.a) 2-40%, 3-33%, 5-30%, 10-25%, or 15-20% of the cells in the population are enucleated;
i.b) greater than 0%, 0.1%, 0.2%, or 0.5%, but less than 2%, 3%, 4%, or 5% of the cells in the population are enucleated;
i.c) greater than 0%, 0.1%, 0.2%, or 0.5%, but less than 6%, 10%, 15%, 20%, or 25% of the cells in the population are enucleated;
i.d) greater than 0%, 0.1%, 0.2%, or 0.5%, but less than 30%, 35%, 40%, 45%, or 50% of the cells in the population are enucleated;
i.e) no more than 1%, 2%, 3%, 5%, 10%, 15%, or 20% of the cells in the population are enucleated;
i.f) no more than 25%, 30%, 35%, 40%, 45%, or 50% of the cells in the population are enucleated;
i.g) the population of cells has reached 6-70%, 10-60%, 20-50%, or 30-40% of maximal enucleation;
i.h) the population of cells has reached no more than 1%, 2%, 3%, 5%, 10%, 15%, or 20% of maximal enucleation;
i.i) the population of cells has reached no more than 25%, 30%, 35%, 40%, 45%, 50%, or 60% of maximal enucleation;
ii.a) the population of cells is fewer than 3, 2, or 1 population doubling from a plateau in cell division;
ii.b) the population of cells is capable of fewer than 3, 2, or 1 population doubling;
ii.c) the population will increase by no more than 1.5, 2, or 3 fold before the population reaches an enucleation level of at least 70% of cells in the population;
iii.a) at least 80%, 85%, 90%, 95%, or 99% of the cells in the population are normoblasts (e.g., polychromatic or orthochromatic normoblasts);
iii.b) at least 50%, 60%, 70%, 75%, or 79% of the cells in the population are normoblasts (e.g., polychromatic or orthochromatic normoblasts);
iii.c) 30-90%, 40-90%, 50-90%, 60-90%, or 70-90% of the cells in the population are normoblasts (e.g., polychromatic or orthochromatic normoblasts);
iii.d) at least 80%, 85%, 90%, 95%, or 99% of the cells in the population exhibit the morphology of a normoblast (e.g., a polychromatic or orthochromatic normoblast);
iii.e) at least 50%, 60%, 70%, 75%, or 79% of the cells in the population exhibit the morphology of a normoblast (e.g., a polychromatic or orthochromatic normoblast); or
iii.f) 30-90%, 40-90%, 50-90%, 60-90%, or 70-90% of the cells in the population exhibit the morphology of a normoblast (e.g., a polychromatic or orthochromatic normoblast).

54. The method of claim 53, wherein the population of erythroid cells or the population of differentiating erythroid cells is a population of erythroid cells comprising a property from i and a property from ii.

55. The method of claim 53, wherein the population of erythroid cells or the population of differentiating erythroid cells is a population of erythroid cells comprising a property from i and a property from iii.

56. The method of claim 53, wherein the population of erythroid cells or the population of differentiating erythroid cells is a population of erythroid cells comprising a property from ii and a property from iii.

57. The method of claim 53, wherein the population of erythroid cells or the population of differentiating erythroid cells is a population of erythroid cells comprising a property from i, a property from ii, and a property from iii.

58. The method of any of claims 43-57, which comprises contacting the cells with the ribonuclease inhibitor at a time when (e.g., by a flow cytometry assay, e.g., a flow cytometry assay of Example 10) the cells comprise one or more (e.g., 2, 3, 4, 5, or more) of the following properties:
84-99%, 85-95%, or about 90% of the cells in the population are GPA-positive;
at least 84%, 85%, 90%, 95%, or 99% of the cells in the population are GPA-positive;
54-99%, 55-98%, 60-95%, 65-90%, 70-85%, or 75-80% of the cells in the population are band3-positive;
at least 54%, 55%, 60%, 65%, 70%, 75%, 80%, 85%, 90%, 95%, 98%, or 99% of the cells in the population are band3-positive;
96-100%, 97-99%, or about 98% of the cells in the population are alpha4 integrin-positive; or
at least 95%, 96%, 97%, 98%, or 99% of the cells in the population are alpha4 integrin-positive.

59. The method of any of claims 43-58, wherein the mRNA is in vitro transcribed mRNA.

60. The method of any of claims 43-59, wherein at least 80%, 85%, 90%, or 95% of the cells of the population are viable 5 days after the cells are contacted with the mRNA.

61. The method of any of claims 43-60, wherein at least 10%, 20%, 30%, 40%, 50%, 60%, 70%, 80%, or 90% of the cells of the population are enucleated 5 days after the cells are contacted with the mRNA.

62. The method of any of claims 43-61, wherein the proportion of cells that are enucleated 5 days after the cells are contacted with the mRNA is at least 50%, 60%, 70%, 80%, 90%, or 95% of the proportion of cells that are enucleated in an otherwise similar population of cells not treated with the ribonuclease inhibitor.

63. The method of any of claims 43-62, wherein the population of cells comprises at least 1×10⁶, 2×10⁶, 5×10⁶, 1×10⁷, 2×10⁷, 5×10⁷, or 1×10⁸ cells at the time the cells are contacted with the mRNA.

64. The method of any of claims 43-63, wherein the population of cells expands by at least 10%, 20%, 30%, 40%, 50%, 60%, 70%, 80%, or 90% within 5 days after the cells are contacted with the mRNA.

65. The method of any of claims 43-64, wherein at least 50%, 60%, 70%, 80%, 85%, 90%, or 95% of the cells of the population express the exogenous protein, e.g., 5 days after the cells are contacted with the mRNA.

66. The method of any of claims 43-65, wherein at least 50%, 60%, 70%, 80%, 85%, 90%, or 95% of the cells of the population comprise at least 1,000, 2,000, 5,000, 10,000, 20,000, 50,000, or 100,000 copies of the exogenous protein, e.g., 5 days after the cells are contacted with the mRNA.

67. The method of any of claims 43-66, wherein the population of cells comprises at least 10%, 20%, 30%, 40%, 50%, 60%, 70%, 80%, or 90% more, or at least 2-fold, 3-fold, 4-fold, or 5-fold more of the exogenous protein than an otherwise similar population of cells not treated with the ribonuclease inhibitor.

68. A reaction mixture comprising: i) an erythroid cell, ii) an mRNA comprising an exogenous protein and iii) a ribonuclease inhibitor.

69. The reaction mixture of claim 68, wherein the mRNA is inside the erythroid cell.

70. The reaction mixture of claim 68 or 69, which comprises a plurality of erythroid cells.

71. A method of assaying a reaction mixture comprising enucleated erythroid cells that comprise an exogenous protein for a ribonuclease inhibitor, comprising:
providing a reaction mixture comprising enucleated erythroid cells that comprise an exogenous protein,
assaying for the presence or level of a ribonuclease inhibitor, e.g., by ELISA, Western blot, or mass spectrometry, e.g., in an aliquot of the reaction mixture.

72. The method of claim 71, further comprising comparing the level of ribonuclease inhibitor to a reference value.

73. The method of claim 72, further comprising responsive to the comparison, one or more of:
classifying the population, e.g., as meeting a requirement or not meeting a requirement, e.g., wherein the requirement is met when the level of ribonuclease inhibitor is below the reference value,
classifying the population as suitable or not suitable for a subsequent processing step, e.g., when the population is suitable for a subsequent purification step when the level of ribonuclease inhibitor is above the reference value,
classifying the population as suitable or not suitable for use as a therapeutic, or
formulating or packaging the population, or an aliquot thereof, for therapeutic use, e.g., when the level of ribonuclease inhibitor is below the reference value.

74. The reaction mixture or method of any of claims 43-73, wherein the ribonuclease inhibitor is RNAsin Plus, Protector RNAse Inhibitor, or Ribonuclease Inhibitor Huma.

75. A method of making an erythroid cell comprising an mRNA that encodes an exogenous protein, comprising:
providing a reaction mixture comprising an erythroid cell and an mRNA encoding the exogenous protein, under conditions which inhibit protein degradation, e.g., by inclusion in the reaction mixture a proteasome inhibitor, and
maintaining the reaction mixture under conditions that allow uptake of the mRNA by the erythroid cell,
thereby making an erythroid cell comprising an mRNA encoding an exogenous protein.

76. The method of claim 75, comprising providing a population of erythroid cells and contacting the population with the mRNA encoding the exogenous protein.

77. The method of claim 75 or 76, wherein a plurality of erythroid cells of the population each takes up an mRNA encoding the exogenous protein.

78. The method of any of claims 75-77, wherein the cell or plurality of cells express the exogenous protein.

79. The method of any of claims 75-78, wherein the cell or plurality of cells comprise the exogenous protein.

80. The method of any of claims 75-79, which further comprises electroporating the cell or population of cells.

81. The method of any of claims 75-80, which further comprises contacting a population of erythroid cells with a proteasome inhibitor.

82. The method of any of claims 75-81, which comprises contacting the population of cells with the proteasome inhibitor before, during, or after contacting the cells with the mRNA, e.g., 0.5-2 days before or after contacting the cells with the mRNA.

83. The method of any of claims 75-82, which comprises contacting the cells with the proteasome inhibitor at day 4, 5, or 6 of maturation phase.

84. The method of any of claims 75-83, wherein the cell is in maturation phase.

85. The method of any of claims 75-84, which comprises contacting the cells with the proteasome inhibitor at a time when the cells comprise one or more (e.g., 2, 3, 4, 5, 6, 7, 8, 9, 10, 11, 12, 13, 14, 15, 16, 17, or more) of the following properties:
i.a) 2-40%, 3-33%, 5-30%, 10-25%, or 15-20% of the cells in the population are enucleated;
i.b) greater than 0%, 0.1%, 0.2%, or 0.5%, but less than 2%, 3%, 4%, or 5% of the cells in the population are enucleated;
i.c) greater than 0%, 0.1%, 0.2%, or 0.5%, but less than 6%, 10%, 15%, 20%, or 25% of the cells in the population are enucleated;
i.d) greater than 0%, 0.1%, 0.2%, or 0.5%, but less than 30%, 35%, 40%, 45%, or 50% of the cells in the population are enucleated;
i.e) no more than 1%, 2%, 3%, 5%, 10%, 15%, or 20% of the cells in the population are enucleated;
i.f) no more than 25%, 30%, 35%, 40%, 45%, or 50% of the cells in the population are enucleated;
i.g) the population of cells has reached 6-70%, 10-60%, 20-50%, or 30-40% of maximal enucleation;
i.h) the population of cells has reached no more than 1%, 2%, 3%, 5%, 10%, 15%, or 20% of maximal enucleation;
i.i) the population of cells has reached no more than 25%, 30%, 35%, 40%, 45%, 50%, or 60% of maximal enucleation;
ii.a) the population of cells is fewer than 3, 2, or 1 population doubling from a plateau in cell division;
ii.b) the population of cells is capable of fewer than 3, 2, or 1 population doubling;
ii.c) the population will increase by no more than 1.5, 2, or 3 fold before the population reaches an enucleation level of at least 70% of cells in the population;
iii.a) at least 80%, 85%, 90%, 95%, or 99% of the cells in the population are normoblasts (e.g., polychromatic or orthochromatic normoblasts);
iii.b) at least 50%, 60%, 70%, 75%, or 79% of the cells in the population are normoblasts (e.g., polychromatic or orthochromatic normoblasts);
iii.c) 30-90%, 40-90%, 50-90%, 60-90%, or 70-90% of the cells in the population are normoblasts (e.g., polychromatic or orthochromatic normoblasts);
iii.d) at least 80%, 85%, 90%, 95%, or 99% of the cells in the population exhibit the morphology of a normoblast (e.g., a polychromatic or orthochromatic normoblast);
iii.e) at least 50%, 60%, 70%, 75%, or 79% of the cells in the population exhibit the morphology of a normoblast (e.g., a polychromatic or orthochromatic normoblast); or
iii.f) 30-90%, 40-90%, 50-90%, 60-90%, or 70-90% of the cells in the population exhibit the morphology of a normoblast (e.g., a polychromatic or orthochromatic normoblast).

86. The method of claim 85, wherein the population of erythroid cells or the population of differentiating erythroid cells is a population of erythroid cells comprising a property from i and a property from ii.

87. The method of claim 85, wherein the population of erythroid cells or the population of differentiating erythroid cells is a population of erythroid cells comprising a property from i and a property from iii.

88. The method of claim 85, wherein the population of erythroid cells or the population of differentiating erythroid cells is a population of erythroid cells comprising a property from ii and a property from iii.

89. The method of claim 85, wherein the population of erythroid cells or the population of differentiating erythroid cells is a population of erythroid cells comprising a property from i, a property from ii, and a property from iii.

90. The method of any of claims 75-89, which comprises contacting the cells with the proteasome inhibitor at a time when (e.g., by a flow cytometry assay, e.g., a flow cytometry assay of Example 10) the cells comprise one or more (e.g., 2, 3, 4, 5, or more) of the following properties:
84-99%, 85-95%, or about 90% of the cells in the population are GPA-positive;
at least 84%, 85%, 90%, 95%, or 99% of the cells in the population are GPA-positive;
54-99%, 55-98%, 60-95%, 65-90%, 70-85%, or 75-80% of the cells in the population are band3-positive;
at least 54%, 55%, 60%, 65%, 70%, 75%, 80%, 85%, 90%, 95%, 98%, or 99% of the cells in the population are band3-positive;
96-100%, 97-99%, or about 98% of the cells in the population are alpha4 integrin-positive; or
at least 95%, 96%, 97%, 98%, or 99% of the cells in the population are alpha4 integrin-positive.

91. The method of any of claims 75-90, wherein the mRNA is in vitro transcribed mRNA.

92. The method of any of claims 75-91, wherein at least 50%, 60%, 70%, 80%, 85%, 90%, or 95% of the cells of the population are viable 5 days after the cells are contacted with the mRNA.

93. The method of any of claims 75-92, wherein at least 10%, 20%, 30%, 40%, 50%, 60%, 70%, 80%, or 90% of the cells of the population are enucleated 5 days after the cells are contacted with the mRNA.

94. The method of any of claims 75-93, wherein the proportion of cells that are enucleated 5 days after the cells are contacted with the mRNA is at least 50%, 60%, 70%, 80%, 90%, or 95% of the proportion of cells that are enucleated in an otherwise similar population of cells not treated with the proteasome inhibitor.

95. The method of any of claims 75-94, wherein the population of cells comprises at least 1×10⁶, 2×10⁶, 5×10⁶, 1×10⁷, 2×10⁷, 5×10⁷, or 1×10⁸ cells at the time the cells are contacted with the mRNA.

96. The method of any of claims 75-95, wherein the population of cells expands by at least 10%, 20%, 30%, 40%, 50%, 60%, 70%, 80%, or 90% within 5 days after the cells are contacted with the mRNA.

97. The method of any of claims 75-96, wherein at least 50%, 60%, 70%, 80%, 85%, 90%, or 95% of the cells of the population express the exogenous protein, e.g., 5 days after the cells are contacted with the mRNA.

98. The method of any of claims 75-97, wherein at least 50%, 60%, 70%, 80%, 85%, 90%, or 95% of the cells of the population comprise at least 1,000, 2,000, 5,000, 10,000, 20,000, 50,000, or 100,000 copies of the exogenous protein, e.g., 5 days after the cells are contacted with the mRNA.

99. The method of any of claims 75-98, wherein the population of cells comprises at least 10%, 20%, 30%, 40%, 50%, 60%, 70%, 80%, or 90% more, or at least 2-fold, 3-fold, 4-fold, or 5-fold more of the exogenous protein than an otherwise similar population of cells not treated with the proteasome inhibitor.

100. A reaction mixture comprising: i) an erythroid cell, ii) an mRNA comprising an exogenous protein and iii) a proteasome inhibitor.

101. The reaction mixture of claim 100, wherein the mRNA is inside the erythroid cell.

102. The reaction mixture of claim 100 or 101, which comprises a plurality of erythroid cells.

103. A method of assaying a reaction mixture comprising enucleated erythroid cells that comprise an exogenous protein for a proteasome inhibitor, comprising:
providing a reaction mixture comprising enucleated erythroid cells that comprise an exogenous protein,
assaying for the presence or level of a proteasome inhibitor, e.g., by ELISA, Western blot, or mass spectrometry, e.g., in an aliquot of the reaction mixture.

104. The method of claim 103, further comprising comparing the level of proteasome inhibitor to a reference value.

105. The method of claim 104, further comprising, responsive to the comparison, one or more of:
classifying the population, e.g., as meeting a requirement or not meeting a requirement, e.g., wherein the requirement is met when the level of proteasome inhibitor is below the reference value,
classifying the population as suitable or not suitable for a subsequent processing step, e.g., when the population is suitable for a subsequent purification step when the level of proteasome inhibitor is above the reference value,
classifying the population as suitable or not suitable for use as a therapeutic, or
formulating or packaging the population, or an aliquot thereof, for therapeutic use, e.g., when the level of proteasome inhibitor is below the reference value.

106. The reaction mixture or method of any of claims 75-105, wherein the proteasome inhibitor is a 20S proteasome inhibitor, e.g., MG-132 or carfilzomib, or a 26S proteasome inhibitor, e.g., bortezomib.

107. A method of making an erythroid cell comprising an mRNA encoding a first exogenous protein and a second exogenous protein, comprising:
a) providing an erythroid cell, e.g., in maturation phase, and
b) contacting the erythroid cell with an mRNA encoding the first exogenous protein and a second mRNA encoding the second exogenous protein, under conditions that allow uptake of the first mRNA and second mRNA by the erythroid cell,
thereby making an erythroid cell comprising the first mRNA and the second mRNA.

108. The method of claim 107, wherein the erythroid cell comprises at least 1,000, 2,000, 5,000, 10,000, 20,000, 50,000, or 100,000 copies of the first exogenous protein and the second exogenous protein, e.g., 5 days after the contacting with the mRNA.

109. A method of producing a population of erythroid cells expressing a first exogenous protein and a second exogenous protein, comprising:
a) providing a population of erythroid cells, e.g., in maturation phase, and
b) contacting the population of erythroid cells with a first mRNA encoding a first protein and a second mRNA encoding a second protein,
thereby making an erythroid cell comprising an mRNA encoding an exogenous protein
wherein at least 80%, 85%, 90%, 95%, 96%, 97%, 98%, or 99% of cells in the population comprise both of the first mRNA and the second mRNA.

110. The method of claim 109, wherein the population of erythroid cells comprises an average of at least 1,000, 2,000, 5,000, 10,000, 20,000, 50,000, or 100,000 copies of the first exogenous protein and the second exogenous protein per cell, e.g., 5 days after the contacting with the mRNA.

111. The method of any of claims 107-110, wherein the contacting comprises performing electroporation.

112. The method of any of claims 109-111, wherein the population of cells comprise the first exogenous protein and the second exogenous protein in at least 80%, 85%, 90%, 95%, 96%, 97%, 98%, or 99% of cells for at least 5 days after the cells were contacted with the first and second mRNAs.

113. A population of erythroid cells wherein at least 80%, 85%, 90%, 95%, 96%, 97%, 98%, or 99% of cells in the population express a first exogenous protein and a second exogenous protein, wherein the population was not made by contacting the cells with DNA encoding the first or second exogenous protein.

114. A method of producing a plurality of erythroid cells comprising a predetermined number of copies of an exogenous protein per cell, comprising contacting the population with a predetermined amount of mRNA encoding the exogenous protein, thereby making the erythroid cell comprising the predetermined amount of the exogenous protein.

115. The method of claim 114, further comprising evaluating one or more of the plurality of erythroid cells (e.g., enucleated erythroid cells) to determine the amount of the exogenous protein.

116. A method of evaluating the amount of an exogenous protein in a sample of erythroid cells, e.g., enucleated erythroid cells comprising:
providing a plurality of erythroid cells comprising a predetermined number of copies of an exogenous protein per cell, which was made by contacting the population with a predetermined amount of mRNA encoding the exogenous protein, and
determining the amount of the exogenous protein in the plurality of erythroid cells.

117. The method of claim any of claims 114-116, wherein:
contacting the cell population with 0.6±20% ug of mRNA per 5E6 cells in the population yields a population of cells expressing 1,000,000±20% copies of the exogenous protein per cell,
contacting the cell population with 0.4±20% ug of mRNA per 5E6 cells in the population yields a population of cells expressing 870,000±20% copies of the exogenous protein per cell,
contacting the cell population with 0.2±20% ug of mRNA per 5E6 cells in the population yields a population of cells expressing 610,000±20% copies of the exogenous protein per cell,
contacting the cell population with 0.1±20% ug of mRNA per 5E6 cells in the population yields a population of cells expressing 270,000±20% copies of the exogenous protein per cell,
contacting the cell population with 0.05±20% ug of mRNA per 5E6 cells in the population yields a population of cells expressing 100,000±20% copies of the exogenous protein per cell, or
contacting the cell population with 0.025±20% ug of mRNA per 5E6 cells in the population yields a population of cells expressing 43,000±20% copies of the exogenous protein per cell.

118. The method of any of claims 114-117, wherein at least 50%, 60%, 70%, 80%, 85%, 90%, or 95% of the cells of the population express the exogenous protein 1 day after the cells are contacted with the exogenous protein.
